(19) United States
(12) Patent Application Publication
(10) Pub. No.: US 2016/0184228 A1
(43) Pub. Date: Jun. 30, 2016

(54) UNILAMELLAR NIOSOMES HAVING HIGH Kow PHARMACOLOGICAL COMPOUNDS SOLVATED THEREIN AND A METHOD FOR THE PREPARATION THEREOF

(71) Applicant: Eric Morrison, West Saint Paul, MN (US)

(72) Inventor: Eric Morrison, West Saint Paul, MN (US)

(21) Appl. No.: 14/985,570

(22) Filed: Dec. 31, 2015

Related U.S. Application Data
(60) Provisional application No. 62/098,382, filed on Dec. 31, 2014.

(51) Int. Cl. A61K 9/127; A61K 9/00; A61K 31/167; A61K 31/192; A61K 31/616

(52) U.S. Cl. CPC A61K 9/1272; A61K 31/192; A61K 31/616; A61K 31/167; A61K 9/0014

(57) ABSTRACT

The present invention provides a niosomal composition having including: one or more high K_{ow} pharmacologically active compounds; one or more water immiscible oils; one or more low HLB surfactants; one or more polyethoxylated high HLB surfactants; water, and wherein the niosomal composition includes an external phase and a dispersed phase, the particles of the dispersed phase including an oil swollen lipid bilayer. Methods of preparing niosomal compositions by hydration of lamellar phase microemulsions and methods of using these compositions to treat various disorders in a patient in need thereof are also provided.

### RELATED APPLICATIONS

This application claims priority to U.S. Provisional Patent Application Ser. No. 62/098,382 filed Dec. 31, 2015, which is hereby incorporated by reference in its entirety for all purposes.

### BACKGROUND OF THE INVENTION

It is often the case that poorly water soluble pharmacological compounds are difficult to administer to living organisms in an effective manner because of one or more problems including poor bioavailability, too rapid decomposition and excretion, which creates a need for frequent re-dosing, and irritation or tissue damage at the location of introduction.

The bioavailability of poorly water soluble, orally administered drug is major challenge for the pharmaceutical industry as many newly launched drugs possess low aqueous solubility, which leads to poor dissolution and low absorption. Furthermore, poor solubility results in variability in absorption and lack of dose proportionality. Compounding the problems of poor absorption is the problem that pharmacologically useful compounds may be substantially degraded in the gastrointestinal tract before absorption can occur. Solutions have been proposed including Self-Emulsifying Drug Delivery Systems (SEDDS's), defined as isotropic mixtures of one or more hydrophilic solvents and co-solvents/surfactants that are capable to form fine oil-in-water (o/w) emulsions upon mild agitation and dilution in gastrointestinal fluids, and various types of emulsions or suspensions,

A further problem of oral drug administration is the potential for harm to the gastrointestinal tract. This problem is most commonly associated with non-steroidal anti-inflammatory drugs (NSAID's). NSAID's diminish the production of prostaglandins by inhibiting cyclooxygenase enzymes, either COX-1 or COX-2 or both. NSAIDs that inhibit COX-1 enzymes interfere with prostaglandin production and blood clotting in the gastrointestinal tract and oral ingestion may lead to gastrointestinal distress and ulceration. For this reason COX-2 inhibitors have been sought as oral medications. However, COX-2 inhibiting drugs are also associated with increased risk of heart attack and stroke and currently celecoxib is the only COX-2 NSAID available in the United States, and it is available only by prescription. Topical administration is a potential solution for COX-1 inhibiting NSAID's including aspirin (ortho-acetyl salicylic acid), ibuprofen (p-isobutyl 2-propenoic acid), and naxopren ((÷)-(S)-2-(6-methoxynaphthalen-2-yl)propanoic acid), which are considered much safer than COX-2 inhibitors and have long been available as over the counter products. Unfortunately, in the United States the only products of these medications are oral forms, and topical products available elsewhere are characterized by poor bioavailability. While it is very desirable to provide COX-1 inhibiting NSAID compounds as topical medications, the only topical NSAID products available in the United States contain diclofenac as the sodium salt dissolved in solvent systems based on propylene glycol, alcohols and dimethylsulfoxide, and are known to cause irritation in significant proportions of patients.

Whether for oral, rectal, intraperitoneal or topical administration, there is a need to improve the usefulness of poorly water soluble high K_{ow} pharmacologically active compounds including NSAID's by improving bioavailability of drug delivery via transmucosal and transdermal routes. For parenteral administration, there is a need to provide effective dispersed forms of poorly water soluble high K_{ow} drugs,

It would be desirable to have liposomal forms of high K_{ow} compounds including NSAID's that are safe, offer high bioavailability, have pharmacologically meaningful concentrations of active compounds, have acceptable organoleptic properties and cosmetically desirable feel, and which are available from simple and reliable manufacturing processes.

### SUMMARY OF THE INVENTION

It has been discovered that stable aqueous unilamellar noisome compositions including pharmacologically meaningful concentrations of high pK_{ow} compounds including NSAID's can be prepared by the decomposition of weakly lamellar microemulsion phases in a simple one step process without the need for high shear mixing. Weakly lamellar microemulsion phases including polyethoxylated surfactants that may occur at relatively higher temperatures can be decomposed by dilution with water or aqueous compositions, by cooling, or both to give a variety of niosome containing product forms, for example, liquids, gels, and yield stress fluids. Physicochemical properties of niosomes, for example, flexibility and adaptability can be optimized to maximize bioavailability of active pharmacological compounds, and compositions free of phospholipids, cholesterol, or both can be prepared.

The present invention provides a niosomal composition including: one or more high K_{ow} pharmacologically active compounds; one or more water immiscible oils; one or more low HLB surfactants; one or more polyethoxylated high HLB surfactants; water, and wherein the niosomal composition includes an external phase and a dispersed phase, the particles of the dispersed phase including a lipid bilayer. Methods of using these compositions to treat various disorders in a patient in need thereof are also provided.

The present invention provides a niosomal composition. The niosomal composition includes: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer.

In one embodiment, the one or more high K_{ow} pharmacologically active compounds include from about 0.05 to about 10 weight percent (%), preferably from about 0.1 to about 7 weight percent (%), and most preferably from about 0.2 to about 5 weight percent (%).

In one embodiment, the one or more water immiscible oils include from about 0.3 to about 70 weight percent (%), preferably from about 0.6 to about 60 weight percent (%), and most preferably from about 1.2 to about 50 weight percent (%).

In one embodiment, the one or more low HLB surfactants include from about 0.15 to about 35 weight percent (%), preferably from about 0.3 about 30 weight percent (%); and most preferably from about 0.6 to about 25 weight percent (%).

In one embodiment, the one or more polyethoxylated high HLB surfactants include from about 0.15 to about 35 weight percent (%), preferably from about 0.3 to about 30 weight percent (%), and most preferably from about 0.6 to about 25 weight percent (%).

In one embodiment, the water includes from about 25 to about 99 weight percent (%), preferably from about 35 to about 98 weight percent (%); and most preferably from about 45 to about 96 weight percent (%).

In one embodiment, the one or more high pharmacologically active compounds each independently include ibuprofen, naproxen, or a combination thereof. In one embodiment, the one or more water immiscible oils each independently include isopropyl myristate, coconut oil, mineral oil, or a combination thereof. In one embodiment, the one or more low HLB surfactants each independently include lecithin, sorbitan monostearate; octanoic acid, or a combination thereof. In one embodiment, the one or more polyethoxylated high HLB surfactants each independently include laureth 23, polysorbate 80, ceteareth-30, or a combination thereof.

The present invention provides a niosomal composition. The niosomal composition includes: one or more high K_{ow} pharmacologically active compounds each independently having a pK_{ow} value greater than about 1,5, wherein the one or more high K_{ow} pharmacologically active compounds each independently include ibuprofen, naproxen, or a combination thereof; one or more water immiscible oils, wherein the one or more water immiscible oils each independently include isopropyl myristate, coconut oil, mineral oil, or a combination thereof; one or more low HLB surfactants each independently having a HLB value of less than 12, wherein the one or more low HLB surfactants each independently include lecithin, sorbitan monostearate; octanoic acid, or a combination thereof; one or more polyethoxylated high HLB surfactants each independently having a HLB value of equal to or greater than 12; wherein the one or more polyethoxylated high HLB surfactants each independently include laureth 23, polysorbate 80, ceteareth-30, or a combination thereof; water, wherein the niosomal composition includes an external phase and a dispersed phase, and wherein one or more particles of the dispersed phase include a lipid bilayer.

In one embodiment, the niosomal composition includes: ibuprofen; isopropyl myristate; laureth 23; sorbitan monostearate; lecithin; mineral oil; and water, or ibuprofen; fractionated coconut oil; laureth 23; lecithin; and water, or ibuprofen; fractionated coconut oil; lecithin; polysorbate 80; and water, or ibuprofen; cholesterol; polysorbate-80; ceteareth-30; lecithin; fractionated coconut oil; and water.

In one embodiment, the niosomal composition includes: naproxen; isopropyl myristate; laureth 23; sorbitan monostearate; lecithin; mineral oil; and water, or naproxen; fractionated coconut oil; laureth 23; lecithin; and water, or naproxen; fractionated coconut oil; lecithin; polysorbate 80; and water, or naproxen; cholesterol; polysorbate-80; ceteareth-30; lecithin; fractionated coconut oil; octanoic acid; and water.

The present invention provides a method of topically treating a disorder in a patient in need thereof. The method includes: administering topically a therapeutically effective amount of a niosomal composition including: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, and wherein the niosomal composition includes an external phase and a dispersed phase.

In one embodiment, the disorder includes pain, inflammation, muscle tightness, muscle spasms, skin ulcerations, scleroderma, eczema, lichen simplex chronicus, rashes, dermatoses, seborrheic dermatitis, psoriasis, atopic dermatitis, or a combination thereof. In one embodiment, the one or more high K_{ow} pharmacologically active compounds each independently include ibuprofen, naproxen, or a combination thereof. In one embodiment, the one or more water immiscible oils each independently include isopropyl myristate, coconut oil, mineral oil, or a combination thereof. In one embodiment, the one or more low HLB surfactants each independently include lecithin sorbitan monostearate; octanoic acid, or a combination thereof. In one embodiment, the one or more polyethoxylated high HLB surfactants each independently include laureth 23, polysorbate 80, celeareth-30, or a combination thereof.

In one embodiment, the niosomal composition includes: ibuprofen; isopropyl myristate; laureth 23; sorbitan monostearate; lecithin; mineral oil; and water, or ibuprofen; fractionated coconut oil; laureth 23; lecithin; and water, or ibuprofen; fractionated coconut oil; lecithin; polysorbate 80; and water, or ibuprofen; cholesterol; polysorbate-80; ceteareth-30; lecithin; fractionated coconut oil; and water.

In one embodiment, the niosomal composition includes: naproxen; isopropyl myristate; laureth 23; sorbitan monostearate; lecithin; mineral oil; and water, or naproxen; fractionated coconut oil; laureth 23; lecithin; and water, or naproxen; fractionated coconut oil; lecithin; polysorbate 80; and water, or naproxen; cholesterol; polysorbate-80; ceteareth-30; lecithin; fractionated coconut oil; octanoic acid; and water.

The present invention provides a niosomal composition. The niosomal composition includes: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer.

In one embodiment, the niosomal composition further includes one or more non-ethoxylated high HLB surfactants. In one embodiment, the external phase includes an aqueous solution. In one embodiment, the one or more high K_{ow} pharmacologically active compounds each independently having a pK_{ow} value greater than about 2.0. In one embodiment, the one or more high K_{ow} pharmacologically active compounds each independently having a pK_{ow} value greater than about 3.0.

In one embodiment, the one or more high K_{ow} pharmacologically active compounds each independently include atropine, cortisol, cortisone, diclofenac, diflusinal, docetaxel, dronabinol, estradiol, flurbiprofen, haloperidol, ibuprofen, ketoprofen, lidocaine, naproxen benzocaine, paclitaxel, penicillin V, prednisone, progesterone, salicylic acid, and sulindac, or a combination thereof.

In one embodiment, the one or more water immiscible oils each independently include one or more hydrocarbons, one or more siloxane polymers, one or more siloxane oligomers, one or more fatty acid esters, one or more polyesters, one or more essential oils, one or more sterols, one or more sterol esters, or a combination thereof. In one embodiment, the water immiscible oil includes a polyester. In one embodiment, the polyester is a triglyceride. In one embodiment, the triglyceride is a saturated and is a liquid at about 20° C. In one embodiment, the triglyceride is fractionated coconut oil or a medium chain triglyceride oil. In one embodiment, the triglyceride is tricaprylin. In one embodiment, the triglyceride is tricaprin. In one embodiment, the one or more hydrocarbons each independently include mineral oil, isoparaffin, isohexadecane, poly(alpha olefins), squalane, squalene, hydrogenated oligomers of propene, butene, and isobutylene cycloaliphatic compounds, and alkylated aromatic compounds, or a combination thereof. In one embodiment, one or more siloxane polymers each independently include cyclopentasiloxane, poly(dimethyl siloxane), phenyl tris(trimethylsiloxy) silane, and poly(methyl phenyl siloxane), or a combination thereof.

In one embodiment, one or more fatty acid esters each independently include fatty acid esters with lower aliphatic alcohols, fatty acid esters with aromatic compounds, fatty acid esters with fatty alcohols, or a combination thereof. In one embodiment, one or more polyesters each independently include fatty acid esters of polyols including sucrose polyesters, trimethylol propane triesters, pentaerythritol and dipentaerythritol polyesters, and glycol or poly(alkylene glycol) diesters, fatty alcohol esters including di and polyacid compounds, or a combination thereof. In one embodiment, the one or more polyesters include propylene glycol dicaprylateldicaprate. In one embodiment, the di and polyacid compounds each independently include phthalic acid, isophthalic acid, trimelletic acid, adipic acid, succinic acid, glutaric acid, citric acid, or a combination thereof. In one embodiment, one or more essential oils each independently include one or more salicylic acid esters, one or more terpenoids, one or more diterpenoids, one or more polyterpenoids, or a combination thereof.

In one embodiment, one or more essential oils each independently include methyl salicylate, geraniol, d-limonene, camphor, menthol, or a combination thereof. In one embodiment, the composition is sprayable. In one embodiment, the composition is a yield stress fluid. In one embodiment, the composition is psuedoplastic. In one embodiment, the one or more low HLB surfactants each independently include one or more mono- and di-esters of glycerin with C₈ to C₂₂ linear or branched, one or more saturated or unsaturated fatty acids, one or more mono-, di- and polyesters of sorbitan with C₈ to C₂₂ linear or branched, saturated or unsaturated fatty acids, one or more mono- and di-esters of ethylene glycol with C₈ to C₂₂ linear or branched, saturated or unsaturated fatty acids, one or more alcohol ethoxylates, one or more alcohol propoxylates, one or more alcohol ethoxylate propoxylates, one or more trialkyl phosphates, one or more phospholipid compounds, one or more phosphate ester compounds formed from esterification of phosphoric acid with short chain polyethoxylates of C₈ to C₂₂ linear or branched, saturated or unsaturated fatty alcohols, one or more aryl alkyl carboxylic acids, one or more aryl alkyl alcohols, one or more saturated or unsaturated, linear or branched aliphatic C₈ to C₂₂ carboxylic acid functional compounds, one or more saturated or unsaturated linear or branched aliphatic C₈ to C₂₂ alcohols, one or more saturated or unsaturated linear or branched aliphatic C₈ to C₂₂ primary and secondary amines and diamines, or a combination thereof.

In one embodiment, the one or more mono- and di-esters of glycerin with C₈ to C₂₂ linear or branched each independently include glycerol monooleate, glycerol monostearate, glycerol dioleate, glycerol distearate, or a combination thereof. In one embodiment, the one or more mono-, di- and polyesters of sorbitan with C₈ to C₂₂ linear or branched, saturated or unsaturated fatty acids each independently include sorbitan monolaurate, sorbitan monopalmitate, sorbitan monostearate, sorbitan monooleate, sorbitan sesquioleate, sorbitan trioleate, sorbitan isostearate, sorbitan tristearate, or a combination thereof. In one embodiment, the one or more mono- and di-esters of ethylene glycol with C₈ to C₂₂ linear or branched, saturated or unsaturated fatty acids each independently include ethylene glycol monooleate, ethylene glycol monostearate, ethylene glycol dioleate, ethylene glycol distearate, or a combination thereof. In one embodiment, the one or more phospholipid compounds each independently include phosphatidyl choline, phosphatidylethanolamine, phosphatidylinositol, or a combination thereof.

In one embodiment, the one or more phosphate ester compounds formed from esterification of phosphoric acid with short chain polyethoxylates of C₈ to C₂₂ linear or branched, saturated or unsaturated fatty alcohols each independently include Rhodafac RP-710, Rhodafac PA32, Lubrhophos LB400, or a combination thereof. In one embodiment, the one or more aryl alkyl carboxylic acids each independently include, or a combination thereof each independently include nonyl oxy benzoic acid, 2-(p-isobutylphenyl)propionic acid (ibuprofen), 2-(6-methoxynaphthalen-2-yl)propanoic acid (naproxen), or a combination thereof. In one embodiment, the one or more aryl alkyl alcohols each independently include nonylphenol, octylphenol, 2,2-dimethyl-3-phenylpropanol (muguet alcohol), phenyl allyl alcohol (cinnamyl alcohol), 8-methyl-N-vanillyl-trans-6-nonenamide (capsaicin), or a combination thereof.

In one embodiment, the one or more saturated or unsaturated, linear or branched aliphatic C₈ to C₂₂ carboxylic acid functional compounds each independently include octanoic acid, coconut fatty acid, oleic acid, ricinoleic acid, stearic acid, carboxylic acid terminated short chain polymers of ricinoleic acid, or a combination thereof. In one embodiment, the one or more saturated or unsaturated linear or branched aliphatic C₈ to C₂₂ alcohols each independently include octanol, dodecanol, myristyl alcohol, ceteryl alcohol, stearyl alcohol, isotridecyl alcohol, 3,7-dimethyl-2,6-octadien-1-ol (nerol), 2-ethyl-1-hexanol, 2-butyl-1-octanol, 2-octyl-1-dodecanol, or a combination thereof. In one embodiment, the one or more saturated or unsaturated linear or branched aliphatic C₈ to C₂₂ primary and secondary amines and diamines each independently include oleyl amine, oleyl diamino propane, cocoalkyl dimethyl amine, or a combination thereof. In one embodiment, the one or more polyethoxylated high HLB surfactants each independently include polyethoxylated sorbitan esters with linear or branched long chain (greater than about 8 carbon atoms) fatty acids, polyethoxylate or polyethoxylate/polypropoxylate ethers with saturated or unsaturated, linear or branched aliphatic C₈ to C₂₂ alcohols, polyethoxylate or polyethoxylate/polypropoxylate esters with saturated or unsaturated, linear or branched aliphatic C₈ to C₂₂ carboxylic acids such, polyethoxylated mono- and di-esters of glycerine with linear or branched long chain (greater than about 8 carbon atoms) fatty acids such, polyethoxylated compounds formed from the addition of ethylene oxide to linear and branched alkylphenol compounds, polyethoxylated castor oils, polyethoxylated compounds formed from the addition of ethylene oxide to amide compounds formed from linear or branched long chain (greater than about 8 carbon atoms) fatty acids, polyethoxylated compounds formed from the addition of ethylene oxide to alcohol functional polysiloxanes, ethylene oxide-propylene oxide block copolymers, or a combination thereof.

In one embodiment, the polyethoxylated sorbitan esters with linear or branched long chain (greater than about 8 carbon atoms) fatty acids include polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), polyoxyethylene (20) sorbitan monooleate (polysorbate 80), or a combination thereof. In one embodiment, the polyethoxylate or polyethoxylate/polypropoxylate ethers with saturated or unsaturated, linear or branched aliphatic C₈ to C₂₂ alcohols include poly(ethylene oxide) octyl ether, poly(ethylene oxide) dodecyl ether, poly(ethylene oxide) myristyl ether, poly(ethylene oxide) ceteryl ether, poly(ethylene oxide) stearyl ether, poly(ethylene oxide) isotridecyl ether, poly(ethylene oxide) 2-ethyl-1-hexanyl ether, poly(ethylene oxide) 2-butyl-1-octyl ether, poly(ethylene oxide) 2-octyl-1-dodecyl ether, or a combination thereof. In one embodiment, the polyethoxylate or polyethoxylate/polypropoxylate esters with saturated or unsaturated, linear or branched aliphatic C₈ to C₂₂ carboxylic acids include poly(ethylene oxide) stearate ester, poly(ethylene oxide) laurate ester, poly(ethylene oxide) oleate ester, or a combination thereof.

In one embodiment, the polyethoxylated mono- and di-esters of glycerine with linear or branched long chain (greater than about 8 carbon atoms) fatty acids include poly(oxyethylene) glyceryl monolaurate, poly(oxyethylene) glyceryl monostearate, or a combination thereof. In one embodiment, the polyethoxylated compounds formed from the addition of ethylene oxide to linear and branched alkylphenol compounds include poly(ethylene oxide) ether with nonyl phenol, poly(ethylene oxide) ether with octyl phenol, or a combination thereof. In one embodiment, the polyethoxylated castor oils include PEG-25 castor oil, PEG-35 castor oil, PEG-40 castor oil, or a combination thereof. In one embodiment, the polyethoxylated compounds formed from the addition of ethylene oxide to amide compounds formed from linear or branched long chain (greater than about 8 carbon atoms) fatty acids include poly(ethylene oxide) ether with coconut acid ethanolamide. I n one embodiment, the polyethoxylated compounds formed from the addition of ethylene oxide to alcohol functional polysiloxanes include poly(ethylene oxide) ether with methyl bis(trimethylsilyloxy)silyl propanol.

In one embodiment, the ethylene oxide-propylene oxide block copolymers include poly(ethylene oxide)-poly(propylene oxide)-poly(ethylene oxide) block copolymers, poly(propylene oxide)-poly(ethylene oxide)-poly(propylene oxide) block copolymers, or a combination thereof.

In one embodiment, the one or more non-ethoxylated high HLB surfactants include polyglyceryl monoesters with linear or branched long chain (greater than about 8 carbon atoms) fatty acids, alkylated mono-, di- and oligoglycosides containing about 8 to about 22 carbon atoms in the alkyl group and ethoxylated alkylated mono-, di- and oligoglycosides containing about 8 to about 22 carbon atoms in the alkyl group, mono- and di-esters of glycerine with linear or branched long chain (greater than about 8 carbon atoms) fatty acids further esterified with short chain monocarboxylic acids, amide compounds formed from linear or branched long chain (greater than about 8 carbon atoms) fatty acids, saturated or unsaturated, linear or branched aliphatic C₈ to C₂₂ alkyl sulfonate and sulfate compounds, sulfonated succinic acid esters with saturated or unsaturated, linear or branched aliphatic C₈ to C₂₂ alcohols, sulfuric acid esters of linear or branched long chain (greater than about 8 carbon atoms) alcohol ethoxylates, alcohol propoxylates, alcohol ethoxylate propoxylates and ethoxylated linear and branched alkylphenol compounds and salts thereof, sulfonates of benzene, cumene, toluene and alkyl substituted aromatic compounds and salts thereof, carboxylates of alcohol ethoxylates, alcohol propoxylates, alcohol ethoxylate propoxylates and ethoxylated linear and branched alkylphenol compounds and salts thereof, long chain (greater than about 8 carbon atoms) acyl amino acids, saturated or unsaturated, linear or branched aliphatic C₈ to C₂₂ alkyl amido propyl (dimethyl ammonio)acetate compounds, sophorolipids, rhamnolipids, or a combination thereof.

In one embodiment, the polyglyceryl monoesters with linear or branched long chain (greater than about 8 carbon atoms) fatty acids include triglycerol monooleate. In one embodiment, the alkylated mono-, di- and oligoglycosides containing about 8 to about 22 carbon atoms in the alkyl group and ethoxylated alkylated mono-, di- and oligoglycosides containing about 8 to about 22 carbon atoms in the alkyl group include poly(D-glucopyranose) ether with (C₈-C₁₄) linear primary alcohols. In one embodiment, the mono- and di-esters of glycerine with linear or branched long chain (greater than about 8 carbon atoms) fatty acids further esterified with short chain monocarboxylic acids include glycerol monostearate lactate. In one embodiment, the amide compounds formed from linear or branched long chain (greater than about 8 carbon atoms) fatty acids include coconut acid diethanolamide, oleic acid diethanolamide, or a combination thereof. In one embodiment, the saturated or unsaturated, linear or branched aliphatic C₈ to C₂₂ alkyl sulfonate and sulfate compounds include octanesulfonic acid, sulfuric acid ester with lauryl alcohol, sulfuric acid ester with lauryl alcohol, or a combination thereof. In one embodiment, the sulfonated succinic acid esters with saturated or unsaturated, linear or branched aliphatic C₈ to C₂₂ alcohols include the bis(2-ethylhexyl)ester of sulfosuccinic acid, the lauryl poly(ethylene oxide) ester of sulfosuccinic acid, or a combination thereof. In one embodiment, the sulfuric acid esters of linear or branched long chain (greater than about 8 carbon atoms) alcohol ethoxylates, alcohol propoxylates, alcohol ethoxylate propoxylates and ethoxylated linear and branched alkylphenol compounds include sodium dodecylpoly(oxyethylene) sulfonate, sodium poly(oxyethylene) octyl phenyl ether sulfonate, or a combination thereof. In one embodiment, the sulfonates of benzene, cumene, toluene and alkyl substituted aromatic compounds include dodecyl benzene sulfonic acid.

In one embodiment, the carboxylates of alcohol ethoxylates, alcohol propoxylates, alcohol ethoxylate propoxylates and ethoxylated linear and branched alkylphenol compounds include poly(ethylene oxide)tridecyl alcohol ether carboxylic acid, sodium poly(ethylene oxide) lauryl ether carboxylate, or a combination thereof. In one embodiment, the long chain (greater than about 8 carbon atoms) acyl amino acids include acyl glutamates, acyl peptides, acyl sarcosinates, acyl taurates, or a combination thereof. In one embodiment, the saturated or unsaturated, linear or branched aliphatic C₈ to C₂₂ alkyl amido propyl (dimethyl ammonio)acetate compounds include lauramidopropyl betaine, stearamidopropyl betaine, or a combination thereof.

In one embodiment, the sophorolipids include mixtures produced by yeasts, for example, Candida bombicola, Candida apicola, Starmerella bombicola, and Candida sp., or a combination thereof. In one embodiment, the Rhamnolipids include compounds produced by Pseudomonas aeruginosa, Burkholderia plantarii, or a combination thereof.

In one embodiment, the niosomal composition consists essentially of: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer.

In one embodiment, the niosomal composition consists of: one or more high K_{ow} pharmacologically active compounds each independently having a pK_{ow} value greater than about 1.5; one or more water immiscible oils; one or more low HLB surfactants each independently having HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer.

The present invention provides a niosomal composition. The niosomal composition includes: ibuprofen; isopropyl myristate; laureth 23; sorbitan monostearate; lecithin; mineral oil; and water. In one embodiment, the niosomal composition consists essentially of: ibuprofen; isopropyl myristate; laureth 23; sorbitan monostearate; lecithin; mineral oil; and water. In one embodiment, the niosomal composition consists of: ibuprofen; isopropyl myristate; laureth 23; sorbitan monostearate; lecithin; mineral oil; and water.

The present invention provides a niosomal composition. The niosomal composition includes: ibuprofen; fractionated coconut oil; laureth 23; lecithin; and water. In one embodiment, the niosomal composition consists essentially of: ibuprofen; fractionated coconut oil; laureth 23; lecithin; and water. In one embodiment, the niosomal composition consists of: ibuprofen; fractionated coconut oil; laureth 23; lecithin; and water.

The present invention provides a niosomal composition. The niosomal composition includes: ibuprofen; fractionated coconut oil; lecithin; polysorbate 80; and water. In one embodiment, the niosomal composition consists essentially of: ibuprofen; fractionated coconut oil; lecithin; polysorbate 80; and water. In one embodiment, the niosomal composition consists of: ibuprofen; fractionated coconut oil; lecithin; polysorbate 80; and water.

The present invention provides a niosomal composition. The niosomal composition includes: naproxen; isopropyl myristate; laureth 23; sorbitan monostearate; lecithin; mineral oil; and water. In one embodiment, the niosomal composition consists essentially of: naproxen; isopropyl myristate; laureth 23; sorbitan monostearate; lecithin; mineral oil; and water. In one embodiment, the niosomal composition consists of: naproxen; isopropyl myristate; laureth 23; sorbitan monostearate; lecithin; mineral oil; and water.

The present invention provides a niosomal composition. The niosomal composition includes: naproxen; fractionated coconut oil; laureth 23; lecithin; and water. In one embodiment, the niosomal composition consists essentially of: naproxen; fractionated coconut oil; laureth 23; lecithin; and water. In one embodiment, the niosomal composition consists of: naproxen; fractionated coconut oil; laureth 23; lecithin; and water.

The present invention provides a niosomal composition. The niosomal composition includes: naproxen; fractionated coconut oil; lecithin; polysorbate 80; and water. In one embodiment, the niosomal composition consists essentially of: naproxen; fractionated coconut oil; lecithin; polysorbate 80; and water. In one embodiment, the niosomal composition consists of: naproxen; fractionated coconut oil; lecithin; polysorbate 80; and water.

The present invention provides a niosomal composition. The niosomal composition includes: ibuprofen; cholesterol; polysorbate-80; ceteareth-30; lecithin; fractionated coconut oil; and water. In one embodiment, the niosomal composition consists essentially of: ibuprofen; cholesterol; polysorbate-80; celeareth-30; lecithin; fractionated coconut oil; and water. In one embodiment, the niosomal composition consists of: ibuprofen; cholesterol; polysorbate-80; ceteareth-30; lecithin; fractionated coconut oil; and water.

The present invention provides a niosomal composition. The niosomal composition includes; naproxen; cholesterol; polysorbate-80; ceteareth-30; lecithin; fractionated coconut oil; octanoic acid; and water. In one embodiment, the niosomal composition consists essentially of: naproxen; cholesterol; polysorbate-80; ceteareth-30; lecithin; fractionated coconut oil; octanoic acid; and water. In one embodiment, the niosomal composition consists of: naproxen; cholesterol; polysorbate-80; ceteareth-30; lecithin; fractionated coconut oil; octanoic acid; and water.

The present invention provides a method of topically treating a disorder in a patient in need thereof. The method includes: administering topically a therapeutically effective amount of niosomal composition including: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, and wherein the niosomal composition includes an external phase and a dispersed phase.

In one embodiment, the disorder includes osteoarthritis, rheumatoid arthritis, hypertension, pain, inflammation, muscle tightness, muscle spasms, skin ulcerations, scleroderma, eczema, lichen simplex chronicus, rashes, dermatoses, seborrheic dermatitis, psoriasis, atopic dermatitis, or a combination thereof.

The present invention provides a method of treating a disorder in a patient in need thereof. The method includes: administering orally a therapeutically effective amount of niosomal composition including: one or more high K_{ow} pharmacologically active compounds each independently having a pK_{ow} value greater than about 1.5; one or more water immiscible oils; one or more low HLB surfactants each independently having a HLB value of less than 12; one or more polyethoxylated high HLB surfactants each independently having a HLB value of equal to or greater than 12; water, and wherein the niosomal composition includes an external phase and a dispersed phase.

In one embodiment, the disorder includes infections, inflammations, cancer, osteoarthritis, rheumatoid arthritis, hypertension, diabetes, or a combination thereof.

The present invention provides a method of topically treating a disorder in a patient in need thereof. The method includes: administering topically a therapeutically effective amount of niosomal composition. The niosomal composition includes: ibuprofen; isopropyl myristate; laureth 23; sorbitan monostearate; lecithin; mineral oil; and water. In one embodiment, the niosomal composition consists essentially of: ibuprofen; isopropyl myristate; laureth 23; sorbitan monostearate; lecithin; mineral oil; and water. In one embodiment, the niosomal composition consists of: ibuprofen; isopropyl myristate; laureth 23; sorbitan monostearate; lecithin; mineral oil; and water.

The niosomal composition includes: ibuprofen; fractionated coconut oil; laureth 23; lecithin; and water. In one embodiment, the niosomal composition consists essentially of: ibuprofen; fractionated coconut oil; laureth 23; lecithin; and water. In one embodiment, the niosomal composition consists of: ibuprofen; fractionated coconut oil; laureth 23; lecithin; and water.

The niosomal composition includes: ibuprofen; fractionated coconut oil; lecithin; polysorbate 80; and water. In one embodiment, the niosomal composition consists essentially of: ibuprofen; fractionated coconut oil; lecithin; polysorbate 80; and water. In one embodiment, the niosomal composition consists of: ibuprofen; fractionated coconut oil; lecithin; polysorbate 80; and water.

The niosomal composition includes: naproxen; isopropyl myristate; laureth 23; sorbitan monostearate; lecithin; mineral oil; and water. In one embodiment, the niosomal composition consists essentially of: naproxen; isopropyl myristate; laureth 23; sorbitan monostearate; lecithin; mineral oil; and water. In one embodiment, the niosomal composition consists of: naproxen; isopropyl myristate; laureth 23; sorbitan monostearate; lecithin; mineral oil; and water.

The niosomal composition includes: naproxen; fractionated coconut oil; laureth 23; lecithin; and water. In one embodiment, the niosomal composition consists essentially of: naproxen; fractionated coconut oil; laureth 23; lecithin; and water. In one embodiment, the niosomal composition consists of: naproxen; fractionated coconut oil; laureth 23; lecithin; and water.

The niosomal composition includes: naproxen; fractionated coconut oil; lecithin; polysorbate 80; and water. In one embodiment, the niosomal composition consists essentially of: naproxen; fractionated coconut oil; lecithin; polysorbate 80; and water. In one embodiment, the niosomal composition consists of: naproxen; fractionated coconut oil; lecithin; polysorbate 80; and water.

The niosomal composition includes: ibuprofen; cholesterol; polysorbate-80; ceteareth-30; lecithin; fractionated coconut oil; and water. In one embodiment, the niosomal composition consists essentially of: ibuprofen; cholesterol; polysorbate-80; ceteareth-30; lecithin; fractionated coconut oil; and water. In one embodiment, the niosomal composition consists of: ibuprofen; cholesterol; polysorbate-80; ceteareth-30; lecithin; fractionated coconut oil; and water.

The niosomal composition includes: naproxen; cholesterol; polysorbate-80; ceteareth-30; lecithin; fractionated coconut oil; octanoic acid; and water. In one embodiment, the niosomal composition consists essentially of: naproxen; cholesterol; polysorbate-80; ceteareth-30; lecithin; fractionated coconut oil; octanoic acid; and water. In one embodiment, the niosomal composition consists of: naproxen; cholesterol; polysorbate-80; ceteareth-30; lecithin; fractionated coconut oil; octanoic acid; and water.

In one embodiment, the disorder includes pain, inflammation, muscle tightness, muscle spasms, skin ulcerations, scleroderma, eczema, lichen simplex chronicus, rashes, dermatoses, seborrheic dermatitis, psoriasis, atopic dermatitis, or a combination thereof.

The present invention provides a niosomal composition. The niosomal composition includes: one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer, with the proviso that the niosomal composition does not contain one or more high K_{ow} pharmacologically active compounds each independently having a value greater than about 1.5.

In one embodiment, the one or more water immiscible oils include from about 0.3 to about 70 weight percent (%), preferably from about 0.6 to about 60 weight percent (%), and most preferably from about 1.2 to about 50 weight percent (%). In one embodiment, the one or more low HLB surfactants include from about 0.15 to about 35 weight percent (%), preferably from about 0.3 to about 30 weight percent (%), and most preferably from about 0.6 to about 25 weight percent (%). In one embodiment, the one or more polyethoxylated high HLB surfactants include from about 0.15 to about 35 weight percent (%), preferably from about 0.3 to about 30 weight percent (%), and most preferably from about 0.6 to about 25 weight percent (%). In one embodiment, the water includes from about 25 to about 99 weight percent 0/0, preferably from about 30 to about 98 weight percent (%), and most preferably from about 40 to about 96 weight percent (%).

In one embodiment, the one or more water immiscible oils each independently include fractionated coconut oil. In one embodiment, the one or more low HLB surfactants each independently include lecithin, cholesterol, celeareth-30, octanoic acid, or a combination thereof. In one embodiment, the one or more polyethoxylated high HLB surfactants each independently include polysorbate 80.

The present invention provides a niosomal composition. The niosomal composition consisting essentially of: one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer, with the proviso that the niosomal composition does not contain one or more high K_{ow} pharmacologically active compounds each independently having a pK_{ow} value greater than about 1.5.

The present invention provides a niosomal composition. The niosomal composition consists of:

one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer, with the proviso that the niosomal composition does not contain one or more high K_{ow} pharmacologically active compounds each independently having a pK value greater than about 1.5.

The present invention provides a niosomal composition. The niosomal composition includes: fractionated coconut oil; ceteareth-30; cholesterol; octanoic acid; polysorbate-80; and water, wherein the niosomal composition includes an external phase and a dispersed phase, and wherein one or more particles of the dispersed phase include a lipid bilayer, with the proviso that the niosomal composition does not contain one or more high K_{ow}, pharmacologically active compounds each independently having a pK_{ow} value greater than about 1.5.

The present invention provides a method of topically treating a disorder in a patient in need thereof. The method includes: administering topically a therapeutically effective amount of a niosomal composition comprising: one or more water immiscible oils; one or more low HLB surfactants each independently having a HLB value of less than 12; one or more polyethoxylated high HLB surfactants each independently having a HLB value of equal to or greater than 12; water, wherein the niosomal composition includes an external phase and a dispersed phase, and wherein one or more particles of the dispersed phase include a lipid bilayer, with the proviso that the niosomal composition does not contain one or more high K_{ow} pharmacologically active compounds each independently having a pK_{ow} value greater than about 1.5.

In one embodiment, the disorder includes pain, inflammation, muscle tightness, muscle spasms, skin ulcerations, scleroderma, eczema, lichen simplex chronicus, rashes, dermatoses, seborrheic dermatitis, psoriasis, atopic dermatitis, or a combination thereof.

### BRIEF DESCRIPTION OF THE DRAWINGS

Embodiments of the invention may be best understood by referring to the following description and accompanying drawings, which illustrate such embodiments. In the drawings:

FIGS. 1-3 represent various flow charts illustrating the preparation of exemplary niosomal compositions.

FIG. 4 is a graph of conductivity (uS/cm) vs. temperature (° C.) for Example 1.

FIG. 5 is a graph of conductivity (uS/cm) vs. temperature (° C.) for Example 2.

FIG. 6 is a graph of conductivity (uS/cm) vs. temperature (° C.) for Example 3.

FIG. 7 is a graph of conductivity (uS/cm) vs. temperature (° C.) for Example 4.

FIG. 8 is a graph of conductivity (uS/cm) vs. temperature (° C.) for Example 5.

FIG. 9 is a graph of conductivity (uS/cm) vs. temperature (° C.) for Example 6.

FIG. 10 is a graph of conductivity (uS/cm) vs. temperature (° C.) for Example 7.

FIG. 11 is a graph of conductivity (uS/cm) vs. temperature (° C.) for Example 8.

FIG. 12 is a graph of conductivity (uS/cm) vs. temperature (° C.) for Example 9.

FIG. 13 is a graph of conductivity (uS/cm) vs. temperature (° C.) for Example 10.

FIG. 14 is a digital photograph of the cryo-TEM of Example 11.

FIG. 15 is a graph of conductivity (uS/cm) vs. temperature (° C.) for Example 13.

FIG. 16 is a graph of conductivity (uS/cm) vs. temperature (° C.) for Example 14.

FIG. 17 is a graph of conductivity (uS/cm) vs. temperature (° C.) for Example 15.

FIG. 18 is a digital photograph of the cryo-TEM of Example 16.

FIG. 19 is a graph of conductivity (uS/cm) vs. temperature (° C.) for Example 17.

FIG. 20 is a digital photograph of the cryo-TEM of Example 18.

FIG. 21 is a graph of conductivity (uS/cm) vs. temperature (° C.) for Example 21.

FIG. 22 is a graph of conductivity (uS/cm) vs. temperature (° C.) for Example 22.

FIGS. 23-24 are graphs of conductivity (uS/cm) vs. temperature (° C.) for Example 23.

FIG. 25 is a graph of conductivity (uS/cm) vs. temperature (° C.) for Example 24.

FIG. 26 is a graph of conductivity (uS/cm) vs. temperature (° C.) for Example 25.

FIG. 27 is a graph of conductivity (uS/cm) vs. temperature (° C.) for Example 26.

The drawings are not necessarily to scale. Like numbers used in the figures refer to like components, steps, and the like. However, it will be understood that the use of a number to refer to a component in a given figure is not intended to limit the component in another figure labeled with the same number.

### DETAILED DESCRIPTION OF THE INVENTION

The present invention provides a niosomal composition having including: one or more high K_{ow} pharmacologically active compounds; one or more water immiscible oils; one or more low HLB surfactants; one or more polyethoxylated high HLB surfactants; water, and wherein the niosomal composition includes an external phase and a dispersed phase, the particles of the dispersed phase including a lipid bilayer. Methods of using these compositions to treat various disorders in a patient in need thereof are also provided.

The present invention provides a niosomal composition having including: one or more high K_{ow} pharmacologically active compounds each independently having a pK_{ow} value greater than about 1.5; one or more water immiscible oils; one or more low HLB surfactants each independently having HLB value of less than 12; one or more polyethoxylated high HLB surfactants each independently having a HLB value of equal to or greater than 12; water, and wherein the niosomal composition includes an external phase and a dispersed phase, the particles of the dispersed phase including a lipid bilayer.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events, which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993 and The American Heritage Dictionary of the English Language, Houghton Mifflin, Boston Mass., 1981.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example, about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “administration” refers to a method of placing a device to a desired site. The placing of a device can be by any pharmaceutically accepted means, for example, by swallowing, retaining it within the mouth until the drug has been dispensed, placing it within the buccal cavity, inserting, implanting, attaching, etc. These and other methods of administration are known in the art.

As used herein, the term “active pharmaceutical ingredient,” or API, refers to a molecular entity adapted for treatment of a malcondition in a patient in need thereof.

As used herein, the term “comprising” or “comprises” is intended to mean that the compositions and methods include the recited elements, but not excluding others.

As used herein, the phrase “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the phrase “consisting of” shall mean excluding more than trace elements of other ingredients and substantial method steps.

As used herein, the terms “consisting essentially of” and “consisting of” are embodied in the term “comprising.”

As used herein, the term “delivery” refers to the release of a drug from a device comprising that drug into an environment surrounding the device. The environment into which the drug so released may or may not be the ultimate site of activity for that drug. In some instances, the released drug may need to be transported to its ultimate site of activity.

As used herein, the term “dermis” refers to the sensitive connective tissue layer of the skin located below the epidermis, containing nerve endings, sweat and sebaceous glands, and blood and lymph vessels. Histologically, the dermis consists of a papillary layer and a reticular layer. The papillary layer contains the vessels and nerve endings supplying the epidermis. The reticular consists predominantly of elastic fibers and collagen.

As used herein, the term “diluent” refers to a pharmacologically inert substance that is nevertheless suitable for human consumption that serves as an excipient in the inventive dosage form. A diluent serves to dilute the API in the inventive dosage form, such that tablets of a typical size can be prepared incorporating a wide range of actual doses of the API.

As used herein, the term “dispersing agent” refers to an agent that facilitates the formation of a dispersion of one or more internal phases in a continuous phase. Examples of such dispersions include suspensions and emulsions, wherein the continuous phase may be water, for example, and the internal phase is a solid or a water-immiscible liquid, respectively. Thus, dispersing agents may include suspending agents and emulsifying agents.

As used herein, the term “dosage form” refers to a physical and chemical composition of an active pharmaceutical ingredient (API) that is adapted for administration to a patient in need thereof. The inventive dosage form is a tablet. By a tablet is meant a relatively hard, compact object, suitable for oral ingestion, prepared by compression of a powder including an active pharmaceutical ingredient and, usually, excipients.

As used herein, the term “dosing event” refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

As used herein, the term “drug” refers to a therapeutic agent or a diagnostic agent and includes any substance, other than food, used in the prevention, diagnosis, alleviation, treatment, or cure of a disease, Stedman's Medical Dictionary, 25^{th} Edition (1990). The drug can include any substance disclosed in at least one of: The Merck Index 13^{th} Edition, 1998, published by Merck & Co., Rahway, N.J.; Pei-Show Juo Concise Dictionary of Biomedicine and Molecular Biology, (1996); U.S. Pharmacopeia Dictionary. 2000 Edition; and Physician's Desk Reference, 2001 Edition.

As used herein, the term “an effective amount” refers to an amount sufficient to effect beneficial or desired results. An effective amount can be administered in one or more administrations, applications, or dosages. Determination of an effective amount for a given administration is well within the ordinary skill in the pharmaceutical arts.

As used herein, the term “epidermis” refers to the outer, protective, nonvascular layer of the skin of vertebrates, covering the dermis. The epidermis consists histologically of five layers, i.e., the stratum corneum, the stratum lucidum, the stratum granulosum, the stratum spinosum, and the stratum basale.

As used herein, the term “essential oil” refers to a volatile oil derived from the leaves, stem, flower or twigs of plants or synthetically-made compounds that have the same chemical attributes. The essential oil usually carries the odor or flavor of the plant. Chemically, each plant essential oil or derivative thereof, which may be extracted from natural sources or synthetically made, generally contains, as a major constituent, an acyclic monoterpene alcohol or aldehyde, a benzenoid aromatic compound containing at least one oxygenated substituent or side chain, or a monocarbocyclic terpene generally having a six-membered ring bearing one or more oxygenated substituents.

As used herein, the term “essential oil” includes derivatives thereof, including racemic mixtures, enantiomers, diastereomers, hydrates, salts, solvates, metabolites, analogs, and homologs

Essential oils, their chemistry and plant families are known in the art. See, for example, S. Price, Aromatherapy Workbook—Understanding Essential Oils from Plant to Bottle, (HarperCollins Publishers, 1993; J. Rose. The Aromatherapy Book—Applications & Inhalations (North Atlantic Books, 1992); and The Merck Index (12th Ed. 1996), each of which is incorporated herein by reference.

As used herein, the term “HLB” refers to Hydrophile-Lipophile Balance, which is an empirical expression for the relationship of the hydrophilic (“water-loving”) and hydrophobic (“water-hating”) groups of a surfactant.

As used herein, the phrase “low HLB surfactant” refers to a surfactant with an HLB value of less than 12.

As used herein, the phrase “high HLB surfactant” refers to a surfactant with an HLB value of equal to or greater than 12.

As used herein, the phrase “High K_{ow} pharmacologically active compounds” refers to useful pharmacologically active compounds that have a pK_{ow} value greater than about 1.5.

As used herein, the term “immersing” refers to dipping, plunging, or sinking into a liquid.

As use herein, the term “immiscible” refers to polymers that will not mix or remain mixed with each other, although at certain conditions, for example, high temperatures, they might mix, but any such mixture will typically be thermodynamically unstable and will typically separate into distinct phases at lower temperatures.

As used herein, the terms “include,” “for example,” “such as,” and the like are used illustratively and are not intended to limit the present invention.

As used herein, the terms “individual,” “host,” “subject,” and “patient” are used interchangeably, and refer to a mammal, including, but not limited to, primates, including simians and humans.

As used herein, the term “infection” refers to the invasion of the host by germs that reproduce and multiply, causing disease by local cell injury, release of poisons, or germ-antibody reaction in the cells. The infection can be in a mammal (e.g., human).

As used herein, the term “liquid” refers to a substance that undergoes continuous deformation under a shearing stress. See, e.g., Concise Chemical and Technical Dictionary, 4^{th} Edition, Chemical Publishing Co., Inc., p. 707, New York, N.Y. (1986).

As used herein, the term “mammal” refers to any of a class of warm-blooded higher vertebrates that nourish their young with milk secreted by mammary glands and have skin usually more or less covered with hair, and non-exclusively includes humans and non-human primates, their children, including neonates and adolescents, both male and female, livestock species, for example, horses, cattle, sheep, and goats, and research and domestic species, including dogs, cats, mice, rats, guinea pigs, and rabbits.

As used herein, the term “miscible” refers to two or more polymeric materials that will form a homogeneous mixture, that is, dissolve in each other. As used herein, the term “molecular weight” refers to a weight-average molecular weight, as is well known in the art.

As used herein, the term “molecular weight” refers to a weight-average molecular weight, as is well known in the art.

As used herein, the term “oil” refers to any of various lubricious, hydrophobic and combustible substances obtained from animal, vegetable and mineral matter. Suitable oils may include petroleum-based oil derivatives, for example, purified petrolatum and mineral oil. Petroleum-derived oils include aliphatic or wax-based oils, aromatic or asphalt-based oils and mixed base oils and may include relatively polar and non-polar oils. “Non-polar” oils are generally oils, for example, petrolatum or mineral oil or its derivatives, which are hydrocarbons and are more hydrophobic and lipophilic compared to synthetic oils, for example, esters, which may be referred to as “polar” oils. It is understood that within the class of oils, that the use of the terms “non-polar” and “polar” are relative within this very hydrophobic and lipophilic class, and all of the oils tend to be much more hydrophobic and lipophilic than the water phase, which is used herein.

As used herein, the terms “optional” or “optionally” mean that the subsequently described event or condition may but need not occur, and that the description includes instances where the event or condition occurs and instances in which it does not.

As used herein, the term “patient” refers to a warm-blooded animal, and preferably a mammal, for example, a out dog, horse, cow, pig, mouse, rat, or primate, including a human.

As used herein, the term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. Several pharmaceutically acceptable ingredients are known in the art and official publications, for example, The United States Pharmcopeia describe the analytical criteria to assess the pharmaceutical acceptability of numerous ingredients of interest.

As used herein, the term “pharmacologically active agent” refers to a chemical compound, complex or composition that exhibits a desirable effect in the biological context, i.e., when administered to a subject. The term includes pharmacologically active, pharmaceutically acceptable derivatives of those active agents specifically mentioned herein, including, but not limited to, salts, esters, amides, prodrugs, active metabolites, isomers, analogs, crystalline forms, hydrates, and the like.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

It will be understood that, although the terms first, second, etc., may be used herein to describe various elements, these elements should not be limited by these terms. These terms are only used to distinguish one element from another. For example, a first element could be termed a second element, and, similarly, a second element could be termed a first element without departing from the teachings of the disclosure.

As used herein, the terms “prevent,” “preventative,” “prevention,” “protect,” and “protection” refer to medical procedures that keep the malcondition from occurring in the first place. The terms mean that there is no or a lessened development of disease or disorder where none had previously occurred, or no further disorder or disease development if there had already been development of the disorder or disease.

As used herein, the term “%” or “percent” refers to weight percent. As used herein, the term “prodrug” refers to any pharmaceutically acceptable form of any of the drugs described herein, which, upon administration to a patient, provides any of the drugs described herein. Pharmaceutically acceptable prodrugs refer to a compound that is metabolized, for example hydrolyzed or oxidized, in the host to form any of the drugs described herein. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The prodrug can be readily prepared from any of the drugs described herein using methods known in the art. See, e.g. See Notari, R. E., “Theory and Practice of Prodrug Kinetics,” Methods in Enzymology, 112:309 323 (1985); Bodor, N., “Novel Approaches in Prodrug Design,” Drugs of the Future, 6(3):165 182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985); Burger's Medicinal Chemistry and Drug Chemistry, Fifth Ed., Vol. 1, pp. 172 178, 949 982 (1995).

The prodrug may be prepared with the objective(s) of improved chemical stability, improved patient acceptance and compliance, improved bioavailability, prolonged duration of action, improved organ selectivity (including improved brain penetrance), improved formulation (e.g., increased hydrosolubility), and/or decreased side effects (e.g., toxicity). See e.g. T. Higuchi and V. Stella, “Prodrugs as Novel Delivery Systems”, Vol. 14 of the A.C.S. Symposium Series; Bioreversible Carriers in Drug Design, ed. Edward B. Roche, American Pharmaceutical Association and Pergamon Press, (1987). Prodrugs include, but are not limited to, compounds derived from any of the drugs described herein wherein hydroxy, amine or sulfhydryl groups, if present, are bonded to any group that, when administered to the subject, cleaves to form the free hydroxyl, amino or sulfhydryl group, respectively. Selected examples include, but are not limited to, biohydrolyzable amides and biohydrolyzable esters and biohydrolyzable carbamates, carbonates, acetate, formate and benzoate derivatives of alcohol and amine functional groups.

The prodrug can be readily prepared from the any of the drugs described herein using methods known in the art. See, for example, Notari, R. E., “Theory and Practice of Prodrug Kinetics,” Methods in Enzymology, 112:309 323 (1985); Bodor, N., “Novel Approaches in Prodrug Design,” Drugs of the Future, 6(3):165 182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985); Burger's Medicinal Chemistry and Drug Chemistry, Fifth Ed., Vol. 1, pp. 172-178, 949-982 (1995).

As used herein, the term “purified” compound refers to a compound that is present in a given quantity at a concentration of at least 50%, 60%, 70%, 80%, 90% and intermediate values thereof and all in weight percent. For example, an isolated compound may be present at 51%, 52%, 53%, 54% and the like, Preferably the compound is present at 90% to 95% and intermediate values thereof. More preferably the compound is present at 95% to 99%, and intermediate values thereof. Even more preferably the compound is present at 99% to 99,9% and intermediate values thereof. Most preferably the compound is present at greater than 99.9% of a given quantity.

As used herein, the term “skin” refers to the external tissue layer in humans and animals consisting of epidermis and dermis.

As used herein, the phrase “room temperature” refers to a temperature in the range of about 20° C. to about 30° C.

As used herein, the phrase “subcutaneous tissue layer” refers to a tissue layer located below the skin. This tissue layer is typically characterized by a loose meshwork of connective tissue, for example, collagen and elastic fibers. It is rich in small vessels, e.g., arterioles and venoles, and capillaries.

As used herein, the term “therapeutic agent” refers to any agent, which serves to repair damage to a living organism to heal the organism, to cure a malcondition, to combat an infection by a microorganism or a virus, to assist the body of the living mammal to return to a healthy state.

As used herein, the term “therapeutic composition” refers to an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use.

As used herein, the term “therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination, Synergy, as described for example, by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, the terms “therapy,” and “therapeutic” refer to either “treatment” or “prevention,” thus, agents that either treat damage or prevent damage are “therapeutic.”

As used herein, the phrase “therapeutic kit” refers to a collection of components that can be used in a medical treatment.

As used herein, the phrase “therapeutic dosage” refers to a dosage considered to be sufficient to produce an intended effect.

As used herein, the phrase “Therapeutically effective modality” refers to a manner in which a medical treatment is performed and is considered to be sufficient to produce an intended effect.

As used herein, the term “tissue” refers to an organized biomaterial usually composed of cells.

As used herein, the term “topically” refers to application of the compositions of the present invention to the surface of the skin and mucosal cells and tissues (e.g., alveolar, buccal, lingual, sublingual, masticatory, or nasal mucosa, and other tissues and cells, which line hollow organs or body cavities).

As used herein, the term “topically active agents” refers to compositions of the present invention that are applied to skin or mucosal surfaces. Desired pharmacological results are intended at or near the site of application (contact) to a subject.

As used herein, the terms “treating” or “treat” or “treatment” refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease.

As used herein, the term “treatment,” covers any treatment of a disease in a mammal, particularly in a human, and includes: (a) preventing the disease from occurring in a subject, which may be predisposed to the disease but has not yet been diagnosed as having it; (b) inhibiting the disease, i.e., arresting its development; and (c) relieving the disease, i.e., causing regression of the disease.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar, and “nm” denotes nanometer.

Concentrations, amounts, etc., of various components are often presented in a range format throughout this disclosure. The description in range format is merely for convenience and brevity and should not be construed as an inflexible limitation on the scope of the claimed invention. Accordingly, the description of a range should be considered to have specifically disclosed all the possible sub ranges as well as individual numerical values within that range. For example, description of a range such as 1% to 8% should be considered to have specifically disclosed sub ranges such as 1% to 7%, 2% to 8%, 2% to 6%, 3% to 6%, 4% to 8%, 3% to 8% etc., as well as individual numbers within that range, such as, 2%, 5%, 7% etc. This construction applies regardless of the breadth of the range and in all contexts throughout this disclosure.

Development of hydrophobic drug nanomedicines is challenging. Much research has been devoted to making nanoparticles containing the hydrophobic anticancer drug paclitaxel. The FDA approved injectible form of paclitaxel is an ethanolic solution of paclitaxel plus nonionic surfactant which dilutes with water to give an aqueous injectible micellular composition that causes anaphylaxis in large numbers of patients.

It has been advised that liposomes are not useful carriers for lipophilic molecules having large oillwater partition coefficients, for example, paclitaxel based upon unsuccessful attempts to load hydrophobic free base doxorubicin into the walls of liposomes (Y Barenholz, “Doxil®—the first FDA-approved nano-drug: lessons learned,” J Control Release, 2012 Jun. 10; 160(2):117-34).

A review of the scientific literature suggests that the capacity of hydrophobic galleries of lipid bilayers for hydrophobic compounds is small. Although lipid bilayers in lyotropic lamellar phases may swell to extremely large spacings (as large as 5000 A) upon addition of oil, oil-swelling tends to separates the bilayer into two monolayers (J. Seddon et al, “Polymorphism of Lipid-Water Systems,” from the Handbook of Biological Physics, Vol. 1, ed. R. Lipowsky, and E, Sackmann, (c) 1995, Elsevier Science B.V. ISBN 0-444-81975-4). Alkanes are reported to partition strongly from lipid bilayers upon contact of alkane containing bilayers with water and the maximum concentration of decane in egg phosphatidyl lipid bilayers in contact with water is less than about 15 weight percent (less than about 0.5 mole fraction: H. Coster et al., “The effect of temperature on lipid-n-alkane interactions in lipid bilayers,” Biochimica et Biophysica Acta, 857 (1986) 95-104).

Including oil in the galleries of liposomes increases entrapment efficiency of hydrophobic drugs and has also been shown to provide other beneficial effects, for example, as dermal penetration. However, small additions of oil cause undesirable changes in liposome structure from small and unilamellar to larger and multilamellar. Larger amounts of oil cause lipid bilayers to decompose to monolayers in the form of filled emulsion droplets.

Incorporation of a small amount of medium chain triglyceride into dimyristoyl phosphatidyl choline liposomes (weight ratio of oil to surfactant=0.04:1) increased the capacity of the liposome for paclitaxel by a factor of nine but also caused the liposomes to become larger with increased lamellarity (S. Hong et al., “Effects of triglycerides on the hydrophobic drug loading capacity of saturated phosphatidylcholine-based liposomes,” International Journal of Pharmaceutics, 483 (2015) 142-150). Including a small amount of cineole, citrol and d-limonene terpene oil mixture in unsaturated soybean phosphatidylcholine liposomes (ratio of oil to surfactant=1:10) improved the dermal penetration of temoporfin by nearly threefold but also caused the liposome morphology to change from small unilamellar liposomes to larger predominantly deformed non-spherical liposomes (N. Dragicevic-Curic et al, “Temoporfin-loaded invasomes: Development, characterization and in vitro skin penetration studies,” J Control Release, 2008 Apr. 7; 127(1):59-69). Sonicating phospholipid liposomes with medium chain triglyceride oil gives filled emulsion droplets (F. Ishii et al., “Properties of various phospholipid mixtures as emulsifiers or dispersing agents in nanoparticle drug carrier preparations,” Colloids and Surfaces B: Biointerfaces, 41 (2005) 257-262).

Poor water solubility of organic compounds including pharmacologically active compounds can result from hydrophobicity and from crystal lattice energy. The degree of hydrophobicity can be characterized by values of the 1-octanol/water partition coefficients, K_{ow} where K_{ow}, for compound X is equal to the molar concentration of compound X in 1-octanol divided by the molar concentration of compound X in water ([X]_{octanol}/[X]_{water}) when the 1-octanol phase and water phase are in equilibrium. K_{ow} values greater than 1 means that the compound is more soluble in 1-octanol than in water. The 1-octanol/water partition coefficient is often expressed as pK_{ow} where pK_{ow}=log₁₀ (K_{ow}). When pK_{ow}=1, the 1-octanol/water partition coefficient is about 10, and the compound can be considered to be hydrophobic or lipophilic. The pK_{ow} values for exemplary pharmacological compounds are shown in Table 1.

TABLE 1
Log K_{ow} Values for Various Pharmaceutical Compounds

| Compound | pK_{ow} |
|---|---|
| acetazolamide | −0.26 |
| ampicillin | −0.81 |
| aspirin | 1.25 |
| atropine | 1.89 |
| baclofen | −0.96 |
| bumetanide | −0.3 |
| cortisol | 1.65 |
| cortisone | 1.47 |
| diclofenac | 1.9 |
| diflusinal | 3.56 |
| dronabinol | 6.97 |
| estradiol | 3.65 |
| flurbiprofen | 3.99 |
| haloperidol | 4.29 |
| ibuprofen | 4.13 |
| ketoprofen | 3.16 |
| ketorolac | 1.26 |
| lidocaine | 2.44 |
| minoxidil | 1.33 |
| naproxen | 3.26 |
| nicotine | 1.18 |
| penicillin V | 2.09 |
| prednisone | 1.89 |
| progesterone | 3.48 |
| salicylic acid | 2.24 |
| sulfacetamide | −0.96 |
| sulindac | 3.24 |
| theophylline | −0.02 |

Useful forms for delivery of poorly water soluble, high K_{ow} pharmacologically active compounds are dispersions of solid or liquid phases including active compounds in an aqueous continuous medium particularly with dispersoid sizes in the tens to hundreds of nanometers. Such dispersoids are referred to as nanocarriers and include liposomes, transfersomes, ethosomes, niosomes, dendrimers, nanoparticles, micellular nanoparticles, and nanoemulsions. Potential benefits of nanocarriers for drug delivery include prolonged duration of action, reduction in the frequency of dosing, more uniform blood plasma levels, reduction of adverse effects, and improvement in bioavailability.

A liposome is an artificially-prepared spherical vesicle composed of a hydrophobic membrane of a lamellar phase lipid bilayer that encapsulates a region of aqueous solution inside. Liposomes are distinct from micelles, reverse micelles, and oil swollen micelles (emulsion droplets), which are composed of monolayers. Liposomes are often composed of phosphatidylcholine-enriched phospholipids. Structurally, niosomes are similar to liposomes, in that they include bilayers. However, in niosomes, surface active compounds in bilayers include non-ionic surfactants in place of or in addition to phospholipids. Both liposomes and niosomes may contain hydrophilic drugs within the aqueous volume enclosed by the lipid bilayer and hydrophobic, high pK_{ow} drugs embedded within the hydrophobic layer made up of the tails of surfactants in the bilayer. Liposomes and niosomes may be multilamellar or unilamellar.

Niosomal compositions including water immiscible oils can include hydrophilic pharmacologically active compounds. In preferred embodiments, the hydrophilic pharmacologically active compounds are water insoluble salts such as doxorubicin sulfate. Water insoluble pharmacologically active compounds can be precipitated in the oil swollen niosome aqueous core by processes described as remote loading which may alternatively be described as active loading. One process for remote loading is the method of pH gradient, where for example a relatively more lipid soluble free base amine form of a drug diffuses through the liposome lipid bilayer where it is protonated to give an ammonium salt which precipitates in the presence of a suitable counter ion (Y Barenholz, “Doxile®—the first FDA-approved nano-drug: lessons learned,” J Control Release, 2012 Jun. 10; 160(2):117-34, doi: 10.1016/j.jconrel.2012.03.020). For example, free base doxorubicin diffuses into the liposome aqueous core and is precipitated with ammonium sulfate to give doxorubicin sulfate.

In preferred embodiments, niosomal compositions including water immiscible oils include macromolecular hydrophilic pharmacologically active compounds including genetic material and vaccines. Macromolecular hydrophilic pharmacologically active compounds can be included in the oil swollen niosome aqueous core by a process described as passive loading. By passive loading it is meant that the pharmacologically active compound is present during formation of the liposome, in which case the liposome may form around the pharmacologically active compound, entrapping it in the aqueous core. For example, passive loading of ovalbumin into cholesterol/dicetyl phosphate/stearate sucrose ester/palmitate sucrose ester niosomes accomplished by hydrating lipids with aqueous buffer containing ovalbumin and removing unencapsulated ovalbumin by ultra filtration gave niosomes that were effective to give increased antibody titres when administered perorally, demonstrating the feasibility of niosomal delivery of vaccines (C. Rental et al., “Niosomes as a novel peroral vaccine delivery system,” International Journal of Pharmaceutics, 186 (1999) 161-167).

In preferred embodiments, macromolecules are passively loaded into oil swollen niosomes by dilution of lamellar phase microemulsions by aqueous compositions including macromolecules. In preferred embodiments, the temperature range of the lamellar phase microemulsions is decreased by inclusion of one or more kosmotropes, and the kosmotropes containing lamellar phase microemulsion is hydrated by dilution with an aqueous composition including macromolecules. In preferred embodiments, a lamellar phase microemulsion is hydrated with an aqueous composition including macromolecules in an isothermal process. In preferred embodiments, a lamellar phase microemulsion including one or more kosmotropes and one or more macromolecules is converted oil swollen liposomes with entrapped macromolecules by dilution with water that does not contain macromolecules.

Physicochemical properties of nanocarrier systems determine the interaction with biological systems and internalization of nanocarriers into cells. The main physicochemical properties that affect cellular uptake are size, shape, rigidity, and electrostatic charge on the surface of nanoparticles. In the case of liposomes, it has been proposed that deformability improves cellular uptake by facilitating movement through biological barriers, for example, the stratum corneum (the upper layer of skin). Research has also provided evidence that interaction between lipids present in liposome lipid bilayer and the stratum corneum can change the structure of the upper skin in a way that favors the penetration of lipophilic drugs.

Niosomes potentially offer advantages over liposomes because the surfactants can be selected for consistency and stability. In the case of liposomes, the mixture of surfactants typically includes phosphatidylcholine-rich phospholipids and may contain small amounts of other molecules while for niosomes the surfactants typically include a large proportion of nonionic surfactants. Nonionic surfactants useful for preparation of niosomes include a wide range of compounds that are stable and available in consistent, non-varying forms, for example, sorbitan fatty acid esters, polyethoxylated sorbitan fatty acid esters, omega-alkyl-bis-(1-aza-18-crown-6) compounds (Bola-surfactants), alcohol ethoxylates formed from the addition of ethylene oxide to fatty alcohols, glyceryl and polyglyceryl fatty acid esters, and saccharide and polysaccharide fatty acid esters. On the other hand, phospholipids, which are required in large amounts for non-niosome liposomes are complex mixtures of natural products that are subject to variability depending upon source and lot. Furthermore, phospholipids, for example, lecithin are chemically unstable because of oxidation of unsaturated fatty acid residues, and in the form of topically applied medicaments, compositions with relatively large amounts of lecithin may exhibit an undesirable sticky or greasy feel.

A problem with liposomes and niosomes is the expense and complexity of synthesis. A very commonly used method of preparation is thin film hydration in which a mixture of surfactants are dissolved in a volatile solvent, for example, diethyl ether, methylene chloride or chloroform and evaporated to give a thin film on the walls of a flask, which is then rehydrated with an aqueous phase. In order to produce unilamellar vesicles or niosomes, it is typical to shear the rehydrated aqueous suspension by sonication, microfluidization, or repeated filtration through nanoporous filters.

Variability in the synthetic methods and difficulty in scaling from laboratory size equipment, for example, rotary evaporators to large scale production makes widespread commercial use of nanocarrier drug delivery systems, for example, as would be appropriate for over the counter product forms, problematic and unacceptably expensive. Use of nanocarriers for drug delivery has typically been restricted to high value applications.

This invention relates to aqueous compositions useful in the delivery of pharmacologically active compounds through biological membranes including skin, blood vessel walls, and mucous membranes. The aqueous compositions include unilamellar niosomes having incorporated therein a pharmacologically active compound, a low HLB surfactant, a polyethoxylated nonionic high HLB surfactant, a water immiscible oil, and optionally cholesterol and/or a phospholipid, for example, lecithin.

The HLB values of various surfactants are listed in Table 2. HLB values were calculated using a proprietary algorithm by Molecular Modeling Pro software, version 5.22, commercialized by Norgwyn Montgomery Software Inc. ©2003. For compounds for which HLB values could not be calculated using MMP software (hydrophile too small), HLB was calculated using the group contribution method of Davies [J. Davies, “A quantitative kinetic theory of emulsion type, I. Physical chemistry of the emulsifying agent,” Gas/Liquid and Liquid/Liquid Interface (Proceedings of the International Congress of Surface Activity (1957)), pp. 426-38] using group contributions from Akzo Nobel Surface Chemistry LLC Technical Information bulletin “HLB & Emulsification-Description of Hydrophile, Lipophile Balance and use of HLB in Producing Emulsions,” Publication SC-11-02, ©2011 Akzo Nobel Surface Chemistry LLC.

TABLE 2
HLB Values of Various Surfactants

| Surfactant | HLB value |
|---|---|
| nonylphenol 20 mol ethoxylate | 16.7 |
| polysorbate-80 | 15.0 |
| polysorbate-20 | 16.7 |
| laureth-23 | 17.9 |
| laureth-30 | 18.6 |
| ceteareth-20 | 16.1 |
| ceteareth-30 | 17.6 |
| PEG-7 glyceryl cocoate | 14.3 |
| isotridecyl 9 mol ethoxylate | 13.8 |
| isotridecyl 30 mol ethoxylate | 18.4 |
| nonylphenol 4 mol ethoxylate | 8.6 |
| nonylphenol * | 3.4 |
| lecithin (American Lecithin Alcolec XTRA-A) ‡ | 2.0 |
| sorbitan stearate | 5.7 |
| laureth-3 | 9.2 |
| glyceryl monostearate | 4.7 |
| dimethyl lauryl amine * | 9.8 |
| isotridecyl 3 mol ethoxylate | 8.6 |
| IB = ibuprofen | 3.0 |
| OA = octanoic acid | 4.5 |
| SA = stearic acid | 1.5 |
| oleic acid | 0.9 |
| NAP = naproxen | 3.5 |
| myristic acid | 2.2 |
| cholesterol | 0.0 |
| lauryl alcohol | 2.1 |
| cetyl alcohol | 1.3 |

HLB values were calculated using Molecular Modeling Pro software, version 5.22, commercialized by Norgwyn Montgomery Software Inc, ©2003 except where noted
* calculated by Davies group contribution method [J. Davies, “A quantitative kinetic theory of emulsion type, I. Physical chemistry of the emulsifying agent,” Gas/Liquid and Liquid/Liquid Interface (Proceedings of the International Congress of Surface Activity (1957)), pp. 426-38] using group contributions from Akzo Nobel Surface Chemistry LLC Technical Information bulletin “HLB & Emulsification-Description of Hydrophile, Lipophile Balance and use of HLB in Producing Emulsions,” Publication SC-11-02, ©2011 Akzo Nobel Surface Chemistry LLC
‡ value from supplier Technical Data Sheet

In preferred embodiments, niosomes are prepared by decomposing micro emulsions occurring at a relatively higher temperature by dilution with water or aqueous compositions, cooling, or both.

It has been known that fine particle size emulsions can be prepared from compositions including polyethoxylated surfactants by processes of phase inversion, including “phase inversion temperature (PIT) emulsification” or “emulsification by PIT.” According to PIT emulsification, phase inversion from a relatively higher temperature water in oil (W/O) emulsion to a relatively lower temperature oil in water (O/W) emulsion occurs for polyethoxylated surfactants because the hydrophilicity of such surfactants changes significantly with temperature. In particular, polyethoxylated surfactants are characterized by relatively lower values of HLB (greater hydrophobicity) at higher temperatures. According to Bancroft's rule, which states that the continuous phase is the phase in which the surfactant has the greatest solubility, cooling emulsions with polyethoxylated surfactants to increase the hydrophilicity of the surfactant is capable to result in phase inversion, in which water transitions from being the internal, discontinuous phase to the continuous phase while oil becomes the dispersed phase. The increased hydrophilicity of polyethoxylated surfactants at lower temperatures is the result of hydration of the polyethoxylate ether oxygen atoms, which are otherwise relatively more hydrophobic, as for example, in dialkyl ether compounds.

Besides the hydrophilicity of the surfactant, the type of emulsion formed depends on several other factors. Typically, when the volume fraction of one phase is very small compared with the other, the phase that has the smaller fraction is the dispersed phase and the other is the continuous phase. Addition of water or aqueous compositions to mixtures of oils and surfactants gives phase inversion at constant temperature as reported, for example, by Forgiarini et al., Langmuir, 2001, 17 (7), 2076-2083. DOI: 10.1021/la001362n. According to Forgiarini et al., the addition of water to a mixture of non-ionic surfactant plus oil gave fine particle size nanoemulsions through a process of inversion without temperature change and without high shear mixing.

In some cases, a system of oil, water and surfactants including polyethoxylated surfactants will not only “invert” as the temperature or composition changes, but will also exhibit intermediate phases including lamellar phases and microemulsion phases. For example, in a PIT emulsification system, a microemulsion phase may exist at temperatures in between the onset and completion of the phase inversion.

Microemulsion formation in the presence of immiscible oil and water phases can be seen as analogous to micelle formation, which occurs in a single phase aqueous system. Surfactants in a single phase aqueous system will dissolve at low concentrations to give dissolved monomer in equilibrium with surfactant assembled at surfaces including the air-water interface and the water-vessel interface. As more surfactant is added to the solution, the available interfacial area becomes increasingly populated with surfactant, and when the water is saturated with surfactant monomer and the interfacial area is completely populated (saturated) with surfactant, the surface tension reaches a minimum and remains constant with further surfactant addition. At the point at which the surface tension plateaus, known as the critical micelle concentration, the surfactant no longer has unpopulated interracial area on which to assemble, and the system responds by creating additional interfacial area through the assembly of surfactant into micelles.

By comparison, in a two phase oil and water system, as the surfactant reaches saturation, instead of creating interfacial area by assembling into micelles, the system may respond by generating interfacial area as a boundary layer between oil and water domains, in which case a microemulsion may form. Micro emulsions can occur at conditions where the surfactant is pushed to create interfacial area as a result of having limited solubility in both oil and water. Depending upon the nature of the oil and surfactants, the temperature range of phase inversion onset and completion is where such solubility conditions exist. If a microemulsion occurs by assembly of surfactant compounds into a boundary layer in the temperature region of phase inversion, it is understood to be thermodynamically stable.

It is possible that the high interfacial area created by self assembly processes in the formation of a microemulsion can be captured to a significant degree by changing system conditions from the point of thermodynamic equilibrium to conditions where an oil in water emulsion is metastable.

Oil in water emulsions prepared by PIT emulsification and high shear processes are known to be metastable with respect to phase separated oil and aqueous phases. Spontaneous decomposition of oil in water emulsions by processes including coalescence and Ostwald ripening may occur so slowly, however, that emulsions can be quite stable, providing acceptably long shelf lives for products containing them. Such stability may be termed kinetic stability or metastability. It is known that coalescence of oil in water emulsions accelerates as system conditions approach phase inversion. It is preferable that compositions prepared by PIT emulsification have a phase inversion point at temperatures greater than about 60° C. and more preferably above about 70° C. or about 80° C.

Previously it has been shown that oil in water emulsions containing high K_{ow}, pharmacological compounds can be prepared by phase inversion methods. For example, PIT emulsification of compositions including ibuprofen was reported by Formiga et al., International Journal of Pharmaceutics, 344 (2007) 158-160. Nanoemulsions were prepared through addition of water to oil plus ibuprofen plus surfactant mixtures by Salim et al., Journal of Nanomedicine and Nanotechnology, 2:113 (2011), DOI:10.4172/2157-7439.1000113.

Recently it has been shown by Lee et al., Langmuir, 2014, Sep. 16; 30(36):10826-33. DOI: 10.1021/la502207f, that decomposition of microemulsion phases can also give oil in water dispersions in which the dispersed phase includes niosomes. Rapid dilution with water and cooling of micro emulsions consisting of nonylphenol, nonylphenol polyethoxylates, hexadecane, and water gave dispersions in which the dispersed oil phase was observed to be vesicular by cryo-TEM.

The process of emulsification described by Lee et al. is a subset of phase inversion processes, for example, PIT emulsification in that it requires a microemulsion phase and that it gives liposomal dispersoids. It was reported that by omitting nonylphenol cosurfactant from the system, an intermediate microemulsion is not obtained, and the dispersion resulting from rapid dilution and cooling is a cloudy emulsion with particle diameter >500 nm. The role of nonylphenol cosurfactant in Lee et al.'s system was to promote microemulsion formation, and it appears that microemulsion formation is in turn related to generation of lamellar structures. The addition of nonylphenol cosurfactant in Lee et al.'s system simultaneously promoted both microemulsion formation and lamellar structure development.

It was further shown by Lee et al. that the extensiveness of lamellae formation in the microemulsion precursor determines the quality of the vesicular dispersion resulting from decomposition. Although micro emulsions containing relatively lower concentrations of nonylphenol were described as most likely being bicontinuous on the basis of freeze fracture cryo-SEM, there was also evidence for lamellae on the basis of slightly depressed electrical conductivity (the percolative pathway through the system has not yet become highly torturous) and the fact that the rate of decrease in electrical conductivity per nonylphenol addition is at a maximum at the point of relatively lower nonylphenol content (the percolative pathway is rapidly becoming torturous due to the presence of non-conducting sheets). Decomposition of the relatively less extensive lamellar systems derived from adding the minimal amount of nonylphenol required for a microemulsion gave small, unilamellar vesicles whereas decomposition of micro emulsions with more nonylphenol gave much larger multilamellar vesicles.

It may be surmised from Lee's measurements of electrical conductivity that nonylphenol increases the both the extensively and rigidity of lamellar structures in systems including water, oil, and surfactants. Although nonylphenol and nonylphenol ethoxylate compounds are useful to provide lamellar phase microemulsions capable to be hydrated to give liposomes, they are unacceptable in pharmacological and personal care products because of the hormone mimetic properties of nonylphenol. Preferable lamellar phase microemulsions and derivative liposomal dispersions are essentially nonylphenol free and contain less than 500 ppm of nonylphenol. It has been found that addition of ibuprofen, like addition of nonylphenol, increases rigidity in lamellar structures, as evidenced by decreased electrical conductivity of micro emulsions.

It is important that rigidity in bilayer structures controlled. Preferable product liposomes have flexible bilayer membranes so as to maximize penetration into the skin and it has been previously described to decrease rigidity in pharmaceutical nanovesicles for topical administration. According to Cevc et al. (U.S. Pat. No. 7,473,432), highly adaptability (via ability to deform, measured as ability to pass through a semi-permeable barrier) in aggregates, for example, vesicles is advantageous for transport through semi-permeable mammalian skin and can result from destabilization of lipid membranes by surfactants including salts of NSAID compounds, Cevc et al. has also noted that mixed lipid vesicles with a highly flexible/fluid membranes are better skin penetrants than much smaller mixed lipid micelles (Cevc et al., Biochimica et Biophysica Acta, 1564 (2002) 21-30). Rigidity in microemulsion lamellar structures can be monitored and controlled using electrical conductivity measurements.

Preferably, the electrical conductivity of higher temperature microemulsion precursors to niosomes is not too greatly depressed relative to the conductivity of final oil in water dispersions (overall conductivities of phases being greatly variable due to the relative presence or absence of ions). In preferred embodiments, the maximum conductivity measured in the microemulsion temperature region of noisome precursor compositions is between about 10% and about 100%, between about 25% and about 99%, and between about 50% and about 95% of the maximum conductivity of the undiluted system measured between about 0° C. and about 100° C.

While the requirement for a pharmalogically meaningful concentration of weakly amphipathic pharmacologically active compound, for example, an NSAID promotes rigidity and extensiveness of lamellar phases to the point that decomposition of micro emulsions to unilarnellar vesicles or the flexibiltty and penetration of vesicles becomes unacceptable, it has been discovered that rigidity may be offset by careful formulation.

Using minimal amounts of bilayer rigidizing compounds, for example, phospholipids including lecithin and cholesterol and using high HLB surfactants with relatively long ethoxylate chains can allow sufficiently weak and non-extensive micro emulsions as indicated by electrical conductivity measurements. Such conductive micro emulsions are useful precursors to small unilamellar vesicles. Furthermore, the flexibility of product niosome can be moderated by the varying the amount of low HLB, lamellae rigidizing compounds.

In preferred embodiments, the concentration of lecithin per non-volatile content of the niosomal composition is less than about 50%, less than about 30%, and less than about 20%. In preferred embodiments, the concentration of phospholipid compounds per non-volatile content of the niosomal composition is less than about 50%, less than about 30%, and less than about 20%. In preferred embodiments, the concentration of elemental phosphorous is less than about 30 mg per gram of non-volatile content of the niosomal composition, less than about 16 mg per gram of non-volatile content of the niosomal composition, and less than about 12 mg per gram of non-volatile content of the niosomal composition. In preferred embodiments, the concentration of cholesterol per non-volatile content of the niosomal composition is less than about 30%, less than about 20%, and less than about 10%. In preferred embodiments, compositions include ethoxylated surfactants with between about 12 and about 100 ethoxylate groups, between about 15 and about 45 ethoxylate groups, between about 20 and about 35 ethoxylate groups.

The extent of lamellar character in micro emulsions and the degree of flexibility of the bilayer membrane in niosomes can also be addressed by choice of water immiscible oil. The choice of water immiscible oil determines the partitioning of the weakly amphipathic pharmacologically active compound between the bulk oil phase and the oil-water interfacial membrane. A useful strategy for controlling the rigidity of niosome bilayers as well as the phase inversion temperature and the temperature range in which micro emulsions occur is to vary the solubility of weakly amphipathic compounds in the oil phase by varying the composition of the oil phase.

In preferred embodiments, the water immiscible oil is selected also on the basis of being capable to promote permeation through skin. For example, numerous sources have reported that isopropyl myristate supports high transdermal fluxes. Panigrahi et al., American Association of Pharmaceutical Scientists PharmSciTech, 6(2): E167-73 (2005), reported that isopropyl myristate gives a pronounced flux enhancing effect for terbutaline sulfate compared to methyl laureate and isopropyl lanolate, which was attributed to solubility parameter of isopropyl myristate being closer to the solubility parameter of human skin. The permeability of alpha tocopherol through human cadaver skin was about 20 to about 100 times greater for an isopropyl myristate solution compared to ethanol solution, light mineral oil solution, or hydroxypropyl cellulose gel (Mahamongkol et al., J. Cosmet. Sci., 56 (2): 91-103 (2005)).

The flux of niosomes across membranes is also a function of the size of the niosomes relative to intercellular pore sizes. It is estimated that the average width of pathways through the skin is about 20-40 nm, and for flexible liposomes useful fluxes have been observed for ratios of liposome diameter to pore diameter of up to about 6 (Cevc et al, Biochimica et Biophysica Acta, 1564 (2002) 21-30). Preferred volume average particle size of niosomes of the present invention are less than about 240 nm, less than about 170 nm, and less than about 120 nm. In preferred embodiments, the particle size of the niosomal composition is measured by static laser light scattering or by dynamic light scattering, for example, using a Horiba 900 series particle size analyzer, a Malvern Zetasizer, a Brookhaven Instruments ZetaPALS DLS instrument, or a Beckman Coulter Delsa Nano C particle size analyzer.

As a constituent of the water immiscible oil, organic compounds may be included for the purpose of increasing transdermal flux of compositions. Such compounds may in some cases be referred to as rubefacients or “rubefacient essential oils” and include compounds that increase blood flow in dermal capillaries. Compounds useful for increasing transdermal flux include salicylates, for example, methyl salicylate and terpenes, for example, geraniol, d-limonene, camphor, and menthol.

In preferred embodiments, compositions of the present invention include ionic surfactants for the purpose of increasing the flux of niosomes across skin. Since the upper layers of skin carry a slight negative surface charge while underlying tissue can carry a much greater negative surface charge (the isoelectric point of the stratum corneum of skin is about 3.7 [Wilkerson, J. Biol. Chem., 1935, 112:329-335], while skin pH is typically in the range of about 4 to about 6 [Ali, Acta Derm Venereol, 2013; 93: 261-267]; mammalian cells have isoelectric points between about 2.1 to about 3.4 [J. Bauer, ed. Cell Electrophoresis, 1994, CRC Press] while the human body internal pH is in the range of about 7 to about 9), there can be a pH and surface charge gradient between the stratum corneum and the underlying epidermis, which is effective to promote transdermal flux of nanoparticles. In preferred embodiments of the present invention, niosomes have isoelectric points above about pH 4, above about pH 6 and above about pH 7. In preferred embodiments, niosomes include a small positive charge measured as zeta potential. The zeta potential is the potential difference between the dispersion medium and the stationary layer of fluid attached to the dispersed niosome surface and is a measurement of the net electrical charge contained within the region bounded by the slipping plane. While relatively higher zeta potential is required for stabilization against coagulation or flocculation in the case of electrostatically stabilized dispersions including as phospholipid based liposomes, relatively lower zeta potential is acceptable for niosomes, for which stabilization results from repulsion due to thermodynamically disallowed reduction of entropy resulting from overlap of nonionic hydrophilic groups. Low zeta potential as is possible for niosomes facilitates transdermal fluxes to the underlying dermal and subdermal layers. In preferred embodiments, the zeta potential of niosomes that include low K_{ow} pharmacological compounds is between about 0 and about 30 mV, between about 1 and about 20 mV, and between about 3 and about 10 mV.

The zeta potential and net electrostatic charge on niosome dispersoids can be moderated by the inclusion of anionic surfactants including phospholipids, phosphate esters, sulfates, sulfonates and deprotonated weak acid amphipathic compounds, by inclusion of cationic surfactants, for example, quaternary ammonium salts, or both. In preferred embodiments, compositions include choline carboxylates, amine functional surfactants, or quaternary ammonium surfactants.

In preferred embodiments, compositions of the present invention including unilamellar niosomes can take the form of sprayable low viscosity liquids, isotonic low viscosity aqueous compositions suitable for parental administration, medium viscosity psuedoplastic liquids. Bingham plastic fluids, and solids. By sprayable it is meant that the composition is capable to be sprayed through dispenser by hand, without the need for high pressure, propellants, sonication or air atomization. The rheological properties including viscosity of the products can be provided by varying the water content, by selection of the oil, surfactants and pharmacological compounds, by the addition of viscosity modifying additives, for example, water soluble or water dispersible polymers or inorganic colloids, or by a combination of these methods.

### Processes

According to the present invention, oil swollen lamellae in lamellar phase microemulsions are hydrated to give small unilamellar liposomes with oil swollen lipid bilayer membranes. Hydration of microemulsions can be the second step that completes phase inversion processes which begin with a first step of transformation of a water in oil emulsion into a microemulsion. Hydration of the hydrophilic moieties in surfactants that make up lipid bilayers in microemulsions causes them to increase in volume, which creates an imbalance of volumes between the hydrophilic moieties and hydrophobic moieties, resulting in bending stress of the surfactant layer at the interface. In particular, volume expansion of the hydrated hydrophilic layer puts it under a compressive stress relative to the hydrophobic layer which is under tensile stress, resulting in curving of the interfacial surfactant layer away from water. Hydration of surfactant interfacial layers including those in lipid bilayers in microemulsions can occur by adding water to the system and also without adding water if hydrogen bonding interaction between water and the surfactant hydrophilic moieties increases. Hydrogen bonding of water to ether oxygen atoms in poly(ethylene oxide) increases as temperature decreases. In preferred processes to convert lamellar phase microemulsions to small unilamellar liposomes, water is not added to the microemulsion in the hydration step. In preferred embodiments, hydration of lamellae in microemulsion is accomplished by adding water to the microemulsion. Addition of water to surfactant composition results in increased chemical activity of water and dilutes salts and other compounds that disrupt hydrogen bonding, and is effective to increase hydration of both ionic and nonionic surfactant moieties.

Hydration of microemulsions by cooling is the final step in phase inversion temperature (PIT) emulsification and hydration of microemulsions by addition of water is the final step in so-called phase inversion composition (PIC) emulsification. For hydration of microemulsions to proceed so as to produce liposomes with oil swollen lipid bilayers instead of filled nanoemulsion droplets, the presence of oil swollen lipid bilayers (lamellae) in microemulsions is a necessary but not sufficient condition. Both the presence and properties of lamellae in microemulsions are critical for allowing the hydration step to occur so as to preserve lipid bilayers in the product. If the microemulsion lamellae are too flexible and components thereof have high mobility, hydration will give filled emulsion droplets or macroscopic phase separation. If microemulsion lamellae are too rigid and coherent, hydration gives unstable dispersions of large multilamellar liposomes. Features of the hydration process are also of note. For example, imbalance in the extent of hydration on opposite sides of lipid bilayers favors curling, while hydration of both sides favors fragmentation which may lead to the formation of flat disc micelles which deform to give spherical micelles. While not wishing to be bound by theory, it is believed that coordination of the curving and the fragmentation of lipid bilayers are of note for the preservation of the lipid bilayer structure and the formation of liposomes.

Imbalance in hydration of opposite sides of lipid bilayers occurs during dilution when water soluble salts or compounds that disrupt hydrogen bonding are diluted preferentially on one side of the lamella relative to the other. In the relative absence of water soluble solutes in the aqueous domains of lamellar phase microemulsions, temperature driven imbalance of hydration across the oil swollen lipid bilayer conceptually becomes increasingly of note. Temperature imbalance and large local temperature gradients result from rapid cooling, and therefore relatively more rapid cooling rates are favorable for the formation of liposomes.

In preferred embodiments of the present invention, lamellar phase microemulsions are converted to liposomes by processes characterized by rapid cooling. In preferred embodiments, microemulsions are converted to liposomes by rapid isocompositional cooling. In preferred embodiments, microemulsions are converted to liposomal dispersions by both cooling and diluting with water.

In preferred embodiments of the present invention, lamellar phase microemulsions contain water soluble salts or compounds that disrupt hydrogen bonding between water and oxygen atoms in polyethoxylated compounds. Water soluble salts and compounds which disrupt hydrogen bonding between water and oxygen atoms in polyethoxylated compounds may be referred to as kosmotropes. The presence of aqueous solutes that disrupt hydrogen bonding between water and polyethoxylate oxygen atoms increase the apparent hydrophobicity of polyethoxylated surfactants, an effect that is similar to dehydration of polyethoxylate oxygen atoms caused by raising the temperature. A water continuous dispersion or emulsion may be converted to a microemulsion by heating, by disrupting hydrogen bonding by including a kosmotrope, or both. Including a kosmotrope is effective to lower the temperature at which a lamellar phase microemulsion occurs, and in preferred embodiments microemulsions include a kosmotrope. In the presence of relatively large concentrations of kosmotropes, lamellar phase microemulsions exist at room temperature and may be converted to liposomal dispersions by isothermal dilution with water. Example 21 shows that addition of glycerin, a nonionic kosmotrope, to a mixture of fractionated coconut oil, polysorbate 80, lecithin, ibuprofen and water such that the weight ratio of water to glycerin was 2/1 was effective to decrease the temperature range at which a lamellar microemulsion phase exists by approximately 12° C. Preferred organic compound kosmotropes include butanol, glycerol, diglycerol, triglycerol, sugars, trehalose, lactic acid, maleic acid, tartaric acid, citric acid and ascorbic acid. Preferred salt kosmotropes include sodium, aluminum, calcium, ammonium, and potassium and magnesium salts of phosphate, sulfate, and hydrogen phosphate ions.

In preferred embodiments of the present invention, lamellar phase microemulsions are converted to liposomal dispersions in isothermal processes, in analogy to the phase change processes that give nanoemulsions such as described by A. Forgiarini et al., “Formation of Nano-emulsions by Low-Energy Emulsification Methods at Constant Temperature,” Langmuir, 2001, 17 (7), 2076-2083) and the method of producing finely divided oil-in-water emulsions described by J. Meyer et al. In United States Patent Application Publication No. 20080004357.

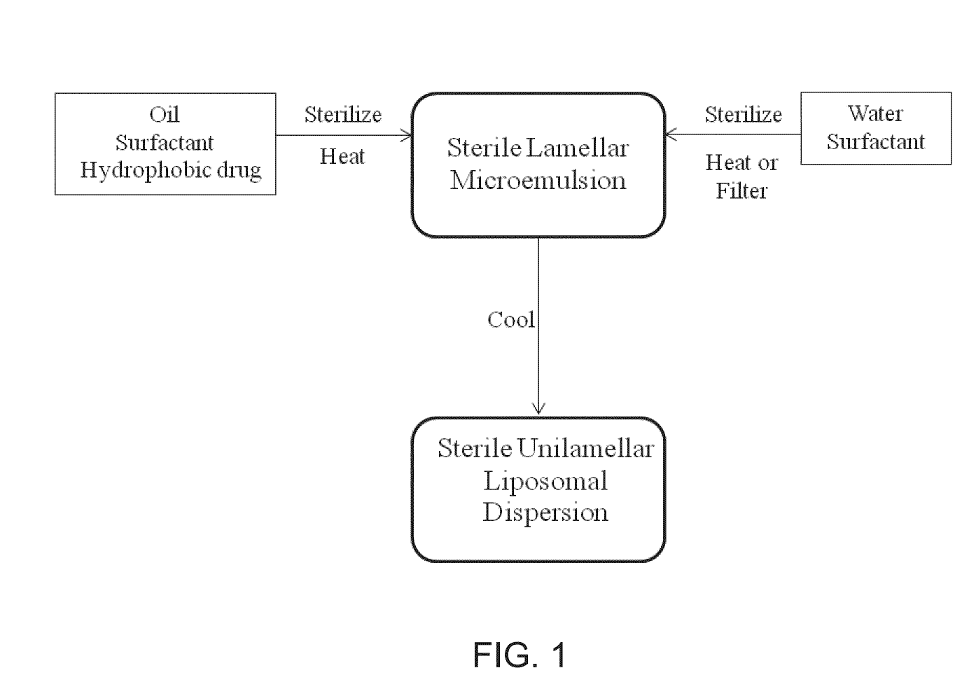

In preferred embodiments of the present invention, liposomal dispersions are prepared by a process as shown in FIG. 1.
1. Preparation a lamellar microemulsion including water, oil, and surfactants
2. Cooling of the microemulsion

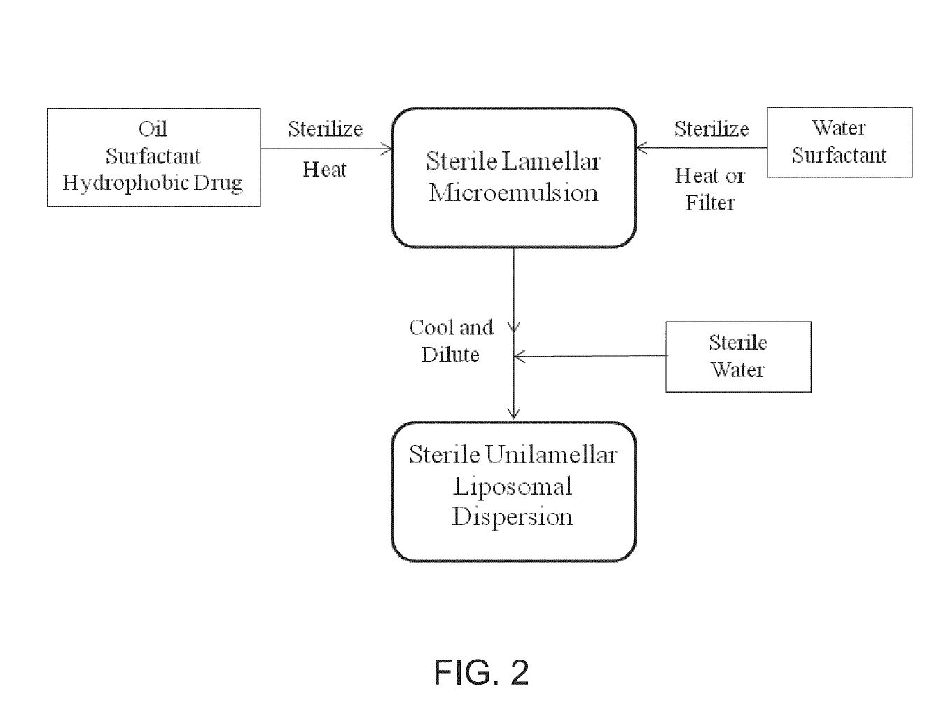

In preferred embodiments of the present invention, liposomal dispersions are prepared by a process as shown in FIG. 2.
1. Preparation a lamellar microemulsion including water, oil, and surfactants
2. Dilution of the microemulsion

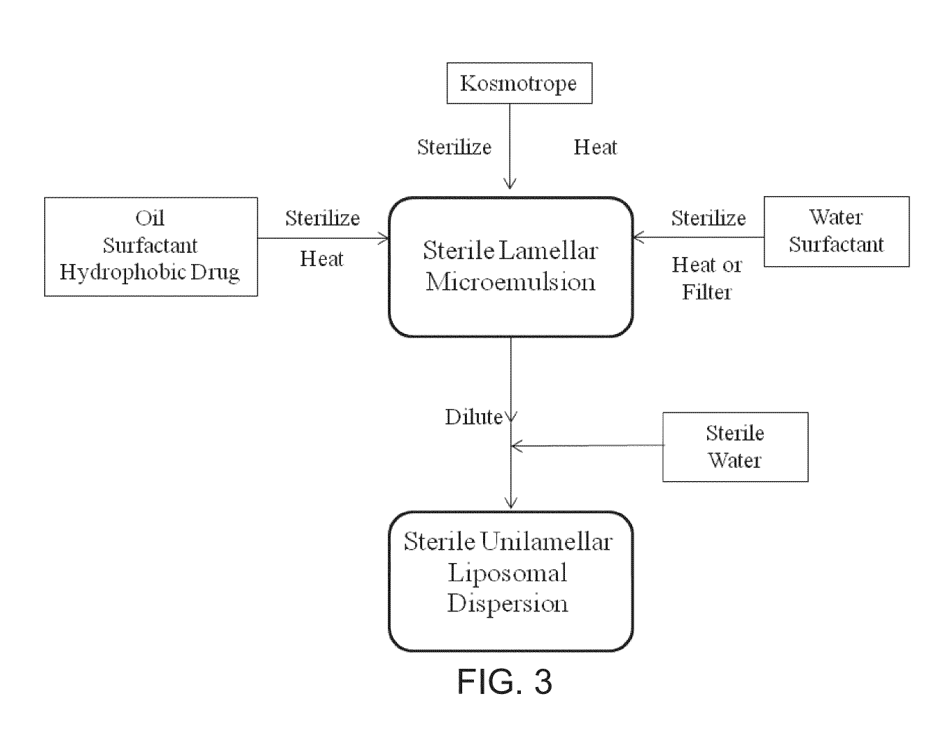

In preferred embodiments of the present invention, liposomal dispersions are prepared by a process as shown in FIG. 3.
1. Preparation a lamellar microemulsion including water, oil, and surfactants
2. Dilution of the microemulsion with water or an aqueous composition wherein the temperature of the water or aqueous composition is lower than the temperature of the microemulsion

In preferred embodiments of the present invention, liposomal dispersions are prepared by a process of:
1. Preparation a lamellar microemulsion including water, oil, kosmotropes and surfactants
2. Cooling of the microemulsion

In preferred embodiments of the present invention, liposomal dispersions are prepared by a process of:
1. Preparation a lamellar microemulsion including water, oil, kosmotropes and surfactants
2. Dilution of the microemulsion

### Compositions

Compositions of the present invention generally include a high K_{ow} pharmacologically active compound, a water immiscible oil, a low HLB surfactant, a polyethoxylated high HLB surfactant, water, and optionally may contain a non-ethoxylated high HLB surfactant and additional components.

### High K_{ow} Pharmacologically Active Compounds

Useful pharmacologically active compounds have a pK_{ow} value greater than about 1.5, preferably greater than about 2.0, and most preferably above about 3.0. Exemplary high pharmacologically active compounds include, atropine, cortisol, cortisone, diclofenac, diflusinal, docetaxel, dronabinol, estradiol, flurbiprofen, haloperidol, ibuprofen, ketoprofen, lidocaine, naproxen benzocaine, paclitaxel, penicillin V, prednisone, progesterone, salicylic acid, and sulindac.

The partitioning of hydrophobic drug between liposomes and water is of note for drug “loading” and “unloading,” For some pharmacological applications of cytotoxic drugs including intravenous and antibiotics and chemotherapeutic anticancer drugs, very high lipid/plasma partition coefficients are desirable to prevent premature drug unloading. Easy dissociation of hydrophobic drugs from liposomes results in pharmokinetics that are relatively unimproved compared to the free drug. For example, an unacceptably low liposome/aqueous medium (plasma) partition coefficient (Kp) for the free base form of doxorubicin and a large 3500-fold dilution upon infusion of the liposomes resulted in fast drug leakage from circulating liposomes, rapid clearance and the same dose-limiting bone marrow toxicity as for free doxorubicin. Incorporation of oil into liposome lipid bilayer will not only increase the capacity for hydrophobic drugs but the liposome/plasma partition coefficient as well. In preferred embodiments, the logarithmic liposome/plasma partition coefficient of pharmacologically active compounds as the base 10 logarithm of moles of pharmacologically active compound situated in a liposome per gram of liposome divided by moles of pharmacologically active compound dissolved in water per gram of water is greater than 3, greater than 4, and greater than 5.

The liposome/plasma partition coefficient of a drug can be modified by chemically altering a pharmacologically active compound to give a derivative that reverts to the active pharmacologically active compound form under physiological conditions. Such a chemically modified pharmacologically active compound may be termed a “prodrug.” An example of derivation of a drug to give a prodrug with a higher octanol/water partition coefficient and lower water solubility is the esterification of paclitaxel and docetaxel with alkoxy silane compounds to give silicate ester derivatives. Wohl et al. report that hydrophobicity and hydrolytic lability of silicates ester derivatives can be independently controlled by varying the alkyl group in tetraalkoxysilaries silicate ester derivatives of paclitaxel and docetaxel, giving prodrugs with four orders of magnitude greater values of the octanol/water partition coefficient (A, WOHL et al., “Silicate Esters of Paclitaxel and Docetaxel: Synthesis, Hydrophobicity, Hydrolytic Stability, Cytotoxicity, and Prodrug Potential,” J. Med. Chem. 2014, 57, 2368-2379). In preferred embodiments, high K_{ow} pharmacologically active compounds include prodrugs.

For transdermal administration of hydrophobic drugs, low liposome/plasma partition coefficients are of note for drug “unloading” after skin permeation. Although it is accepted that liposomes provide improved permeability of drugs through the upper barrier layer of skin (stratum corneum), it is often the case that assays of drugs in the aqueous receiver compartment of Franz diffusion cells show drug concentrations that are negligible and below therapeutic thresholds. Partitioning of weakly acidic and weakly basic amphipathic drugs including carboxylic acid functional NSAID's is governed by complex equilibria including association of both the free and ionized drug with liposomes, acid dissociation, and micellular aggregation of ionized drug. In preferred topical liposomal NSAID compositions, equilibrium that favors aqueous drug solubilization as ionized aqueous monomers or micelles over liposomal solubilization of unionized drug is indicated by apparent pKa values below physiological pH. As shown by Kanicky et al.,“ Effect of Premicellar Aggregation on the pKa of Fatty Acid Soap Solutions,” Langmuir, 2003, 19 (6), pp 2034-2038 DOI: 10.1021/la020672y, apparent pKa values of carboxylic acid compounds are dependent upon intermolecular associations in the dispersed phase. In preferred inventive liposomal medicines for topical application, the composition of the dispersed phase is selected so that the apparent pKa value of carboxylic acid functional pharmacologically active compounds contained therein is less than about 7.4, less than about 7.0, and less than about 6.6. Conversely, preferred inventive basic liposomal medicines for topically application exhibit apparent pKb values for amine functional pharmacologically active compounds above about 7.4, above about 7.8, and above about 8.2. Example 24 demonstrates the determination of the apparent pKa value of ibuprofen. The apparent pKa value in the system is greater than the negative logarithm of the acid dissociation constant for ibuprofen (reported values between 4.9 and 5.2) because the ionization of ibuprofen is governed by partitioning of free acid and ionized forms as well as acidity of the molecule. The apparent pKa value of ibuprofen determined in Example 24 indicates a relatively small lipid/water partition coefficient for ibuprofen, desirable for drug unloading in transdermal drug delivery.

### Hydrohobic Inorganic Nanoparticles

Preferred liposomal dispersions of the present invention include hydrophobic inorganic nanoparticles. Incorporation of hydrophobic nanoparticles into liposomes is of note for renewable energy applications, biological imaging, and targeting of therapeutic action. Useful hydrophobic inorganic nanoparticles are dispersible in hydrophobic oils and include a smaller than about 10 nm core of an inorganic compound which provides a valuable optical, physical and magnetic property encapsulated by hydrophobic groups. For example, magnetoliposomes (magnetic liposomes) including mixed valence Fe(II)/Fe(III) oxide magnetite particles have been investigated for imaging and for targeted drug carrying by guiding and retaining drugs at the target site with the help of an external magnetic field, see, for example, A. Ito, et al., “Medical Application of Functionalized Magnetic Nanoparticles,” Journal of Bioscience and Engineering, Vol. 100, No. 1, 1-11. 2005. Sub 10 nm particles of semiconductor compounds, for example, CdS, PbS, CdSe, PbSe, InAs, and InP (“quantum dots”) are useful probes for cancer markers in fluids are useful as high resolution contrast agents for medical imaging. Hydrophobic quantum dots can be incorporated within phospholipid membranes of liposomes, see, for example, W. Zheng et al., “Quantum Dots Encapsulated within Phospholipid Membranes: Phase-Dependent Structure, Photostability, and Site-Selective Functionalization,” J. Am. Chem. Soc., 2014, 136 (5), pp 1992-1999 DOI: 10.1021/ja411339f. Preparation of liposomal compositions including mixed valence Fe(II)/Fe(III) oxide magnetite is illustrated in Example 25.

### Cryoprotectants

In preferred embodiments, liposomal compositions of the present invention are preserved by lyophilization. Preferred liposomal dispersions of the present invention include cryoprotectants, defined as substances that protect biological tissue from freezing damage (i.e., that due to ice formation). Useful cryoprotectants include, for example, sugars (mono-, di-, and polysaccharides including trehalose and sucrose and polyalcohols. As the mechanisms of lowering the temperature of range of microemulsion and of cryoprotection both involve disruption of hydrogen bonding, compounds may act both as kosmotropes and cryoprotectants. In preferred embodiments, cryoprotectant compounds are incorporated into liposome precursor microemulsions where they serve as kosmotropes and remain in the liposomal dispersion as cryoprotectants. Particularly preferred cryoprotectants include glycerin, trehalose and sucrose.

### Water Immiscible Oils

Useful oils are low volatility water immiscible compounds or mixtures of compounds that are liquid at 20° C. Preferably, oils have sufficiently low volatility so as to provide liposomal compositions that are not flammable and that can be safely manufactured. Fire hazards are reduced if processing is done at temperatures below the flash point of microemulsions. In preferred processes the single compound or mixture of compounds that makes up the oil has a flash point above the formation temperature of the microemulsion, and more preferably above the boiling point of water. Preferred oils, whether a single compound or mixture of compounds, have flash points above about 75° C., above about 100° C., and above about 125° C. Volatile oils which themselves have unacceptably low flash points can be included so long as they are blended with less volatile oils and the flash point of the oil blend is above about 75° C., above about 100° C., or more preferably above about 125° C.

Preferred oils support the formation of lamellae in microemulsion precursors to liposomes. The lamellar character of microemulsions depends upon properties of oil as well as surfactants and composition ranges. As shown in Example 22, the propensity of oil-water-surfactant compositions towards lamellar character increases with decreasing oil molecular weight and increasing oil polarity. In preferred embodiments, the molecular weight of oil (taken as the weight average molecular weight in the case of mixtures of compounds) is sufficiently low that lamellae formation occurs, but is sufficiently large so as to provide low volatility and low flash points. In preferred embodiments, the molecular weight of oil is less than about 900 g/mol, less than about 700 g/mol, and less than about 550 g/mol. In preferred embodiments, the weight average molecular weight of individual water insoluble compounds in oil is less than about 900 g/mol, less than about 700 g/mol, and less than about 550 g/mol.

Preferred oils include, for example, terpenoid compounds, defined as natural compounds derived from isoprene units and containing multiples of five carbon atoms, including oxygenated compounds. Exemplary terpenoid compounds are cyclic and acyclic and include limonene, menthol, carvone, pinene, camphor, cineole, linalool, citronellol, geraniol, and patchoulol. Terpenoid compounds may be added to liposomal compositions as discrete compounds or as an extracted mixture from plants called essential oils. Preferred oils include essential oils extracted from plants. Preferred liposomal compositions include fragrant hydrophobic compounds including both terpenoid and non-terpenoid compounds. In preferred embodiments, liposomal compositions include greater than about 500 ppm by weight of hydrophobic organic compounds with odor thresholds less than about 500 parts per billion by volume. In preferred embodiments, the non-water ingredients of liposomal compositions include greater than about 0.1 percent, greater than about 0.5 percent, and greater than about 1.0 percent by weight of hydrophobic organic compounds with odor thresholds less than about 500 parts per billion by volume. Useful fragrant hydrophobic organic compounds include esters, for example, ethyl butanoate, octyl acetate, and isoamyl acetate; lactones, for example, γ-nonalactone and γ-decalactone; aromatics, for example, cinnamaldehyde, eugenol, anisole, and vanillin; aldehydes, for example, benzaldehyde and hexanal; and ketones, for example, undecanone. In preferred embodiments, hydrophobic fragrance compounds in liposomal compositions exist within the liposome lipid bilayer. When lamellar phase microemulsion compositions include fragrance compounds including terpenoids, it is preferred that flash point of the oil remains above about 75° C., above about 100° C., and above about 125° C. and the ratio of oil to surfactant remains between about 0.12:1 to about 3.5:1, between about 0.24:1 to about 2.5:1, and between about 0.48:1 to about 2.0:1.

Suitable Water Immiscible Oils Include:

(1) Hydrocarbons, for example, mineral oil, isoparaffin, isohexadecane, poly(alpha olefins), squalane and squalene, hydrogenated oligomers of propene, butane, and isobutylene, cycloaliphatic compounds, and alkylated aromatic compounds, for example, alkylated naphthalenes

(2) Siloxane polymers and oligomers, for example, cyclopentasiloxane, poly(dimethyl siloxane), and poly(methyl phenyl siloxane)

(3) Monoesters including fatty acid esters with lower aliphatic alcohols methanol, ethanol and isopropanol; fatty acid esters with aromatic compounds, for example, benzoic acid; fatty acid esters with fatty alcohols. Useful fatty acid esters with lower aliphatic alcohols include methyl decanoate, methyl myristate, isopropyl myristate, and isopropyl palmitate. Useful fatty acid esters with fatty alcohols includes cetyl palmitate and decyl oleate. A particularly preferred monoester is isopropyl palmitate.

(4) Polyesters including: fatty acid esters of polyols including triglycerides, sucrose polyesters, trimethylol propane triesters pentaerythritol and dipentaerythritol polyesters, and glycol or poly(alkylene glycol) diesters; and fatty alcohol esters of di and polyacid compounds, for example, phthalic, isophthalic, trimelletic, adipic, succinic, glutaric, and citric acid. Useful triglycerides include vegetable oils, for example, coconut oil, fractionated coconut oil, sunflower seed oil, olive oil, and canola oil and synthetic triglycerides, for example, medium chain triglyceride (MCT) oil, tricaprin, tridecanoin, triolein, and tristearin. Useful glycol diesters include propylene glycol esters, for example, propylene glycol dicaprylate/dicaprate and propylene glycol dimyristate. Particularly preferred polyesters are saturated and are liquids at about 20° C. and include fractionated coconut oil and propylene glycol dicaprylate/dicaprate.

(5) Essential oils including salicylic acid esters, terpenoids, diterpenoids and polyterpenoids and derivatives thereof including methyl salicylate, geraniol, d-limonene, camphor, and menthol.

(6) Sterols and sterol esters, for example, lanolin.

(7) Ethylenically unsaturated compounds, including mono-, di- and poly-functional water immiscible compounds. Exemplary ethylenically unsaturated compounds include ally acrylate, ethyl acrylate, pentaerythritol triacrylate, pentaerythritol tetraacrylate, 2-t-butyl cyclohexanol acrylate, 1,6-hexanediol diacrylate, stearyl acrylate, behenyl acrylate, isobornyl acrylate, isooctyl acrylate, isotridecyl acrylate, lauryl acrylate, 1,10-decanediol diacrylate, methyl methacrylate, 2-t-butyl cyclohexanol methacrylate, 1,12-dodecanediol dimethacrylate, and lauryl methacrylate.

In preferred embodiments of the present invention, oils are selected from those listed in the United States Food and Drug Administration list of Inactive Ingredients. Preferred oils are those that have been approved for intravenous administration. Preferred oils include soybean oil, safflower oil, tricaprylin, tricaprin, and medium chain triglyceride oils which may also be referred to as caprylic/capric triglycerides and fractionated coconut oil.

### Surfactants

Small unilamellar oil containing liposomes of the present invention are characterized by having at least two surfactants. The precursors to oil containing lipid bilayers in liposomes are oil containing lipid bilayers in microemulsions, and formation of lipid bilayers in microemulsions requires polydispersity in the HLB (hydrophilic/lipophilic balance) values of surfactants which is not possible with a single surfactant.

The physicochemical properties of microemulsion lipid bilayers are critical to the formation of small unilamellar liposomes. If the lipid bilayers are too fluid or flexible, or if mobility of individual components is too high, hydration of the microemulsion results in destruction of the lipid bilayer into monolayers giving micelles or oil filled micelles (nanoemulsion droplets). If microemulsion lipid bilayers are too durable, hydration of the microemulsion will give large multilamellar liposomes. A significant problem in preparing liposomes from microemulsions that do not contain nonylphenol compounds is generating lipid bilayers with sufficient robustness so as to not decompose to monolayers upon hydration.

It has been found that formation of lipid bilayers in microemulsions requires a minimum value of polydispersity in the surfactant HLB values and that the degree of lamellar character in precursor microemulsions can be systematically increased by increasing the HLB polydispersity. In the scientific literature, there are no conventions for expressing polydispersity in HLB values. As it relates to lamellar phase microemulsions, HLB polydispersity can be conveniently expressed as a dimensionless weight mean square deviation, WMSD_{HLB}, analogous to the sum of least squares differences used to express goodness of fit (for example, linear correlation coefficient). WMSD_{HLB} is calculated as the sum of the product of the weight fraction of the i-th surfactant species times the square of the deviation of the HLB of the i-th surfactant species from the weight average HLB normalized by dividing by the weight average HLB according to equation (1) (where weight average HLB=HLBw=ΣᵢwᵢHLBᵢ)

$${WMSD}_{HLB} = \frac{{\sum }_{i} {{w}_{i} ( {HLB}_{i} - {HLB}_{w} )}^{2}}{{HLB}_{w}} \qquad ( 1 )$$

The conventional basis for surfactant HLB values is the Griffin's equation (Griffin W, Calculation of HLB Values of Non-Ionic Surfactants, J. Soc. Cosm. Chem. 5 (4): 249-56) which may be used to calculate HLB for simple ethoxylated surfactants. A more sophisticated method of calculating HLB values is Davies group contribution method for surfactants including groups with known group contribution factors (Davies, A quantitative kinetic theory of emulsion type, I. Physical chemistry of the emulsifying agent, Gas/Liquid and Liquid/Liquid interface, Proc. Int. Congress of Surface Activity, 426-38). The usefulness of Davies' method can be extended by additional group contribution factors, as for example, are provided in Akzo Nobel Surface Chemistry LLC Technical Information bulletin “HLB & Emulsification-Description of Hydrophile, Lipophile Balance and use of HLB in Producing Emulsions,” Publication SC-11-02, ©2011 Akzo Nobel Surface Chemistry LLC.

Because Neither Griffin's or Davies' methods for calculating HLB values are useful for all surfactant types and it is typical to use Griffin's equation for some types and Davies' for others, a more comprehensive method for calculating HLB is of note. Preferably. HLB values for surfactants are calculated using molecular modeling software, for example, Molecular Modeling Pro software, version 5.22, commercialized by Norgwyn Montgomery Software Inc. ©2003. In some cases it is necessary to use surfactant manufacturer provided HLB values, for example, in the case of mixtures of surfactants, for example, as lecithins. In some cases the hydrophilic group is too small for molecular modeling to recognize the molecule as a surfactant, in which case Davies' group contribution method is preferred. Amphipathic drugs may influence properties of lamellae in microemulsions and therefore HLB values for hydrophobic drugs with surfactant properties should be included in calculations of HLB polydispersity. Amphipathic hydrophobic drugs include compounds with carboxylic acid groups, amine groups, and hydroxyl groups.

In preferred embodiments of the present invention, WMSD_{HLB} is greater than about 2, greater than about 3, and greater than about 5.

The susceptibility of mixtures of water and surfactants to form lamellar phases upon introduction of oil depends upon the ratio of low HLB surfactants to high HLB surfactant. As shown in Example 23, the susceptibility of a mixture of surfactants towards forming lamellar microemulsion phases increases as the weight ratio of low HLB surfactant to high HLB surfactant increases from about 0.75:1 to about 1.1. In preferred embodiments of the present invention, liposomal compositions have weight ratios of low HLB surfactants to high HLB surfactants greater than about 0.5, greater than about 0.75, and greater than about 1:1.

In preferred embodiments of the present invention, surfactants are selected from those listed in the United States Food and Drug Administration list of Inactive Ingredients. Particularly preferred surfactants are those that have been approved for intravenous administration. Particularly preferred surfactants include phospholipids; 1,2-dimyristoyl-SN-glycero-3-(phosph-S-(1-glycerol)); 1,2-dimyristoyl-SN-glycero-3-phosphocholine; egg phospholipids; N-(carbonyl-methoxypolyethylene glycol 2000)-1,2-distearolyl-SN-glycero-3-phosphoethanolamine; PEG vegetable oil; PEG-40 castor oil; PEG-60 hydrogenated castor oil; polyoxyethylene fatty acid esters; polyoxyl 35 castor oil; polysorbate 20; polysorbate 40; polysorbate 80; sodium cholesteryl sulfate; sodium desoxycholate; and polyethyleneglycol 660 12-hydroxystearate).

### Low HLB Surfactants

Suitable low HLB surfactants include:

(1) mono- and di-esters of glycerin with C₈ to C₂₂ linear or branched, saturated or unsaturated fatty acids, for example, glycerol monooleate, glycerol monostearate, glycerol dioleate, glycerol distearate, and mixtures of these surfactants;

(2) mono-, di- and polyesters of sorbitan with C₈ to C₂₂ linear or branched, saturated or unsaturated fatty acids, for example, sorbitan monolaurate, sorbitan monopalmitate, sorbitan monostearate, sorbitan monooleate, sorbitan sesquioleate, sorbitan trioleate, sorbitan isostearate, sorbitan tristearate, and mixtures of these surfactants. A particularly preferred sorbitan ester is sorbitan monostearate.

(3) mono- and di-esters of ethylene glycol with C₈ to C₂₂ linear or branched, saturated or unsaturated fatty acids, for example, ethylene glycol monooleate, ethylene glycol monostearate, ethylene glycol dioleate, ethylene glycol distearate, and mixtures of these surfactants.

(4) alcohol ethoxylates, alcohol propoxylates, and alcohol ethoxylate propoxylates formed from the addition of ethylene oxide and/or propylene oxide to C₈ to C₂₂ linear or branched, saturated or unsaturated alcohols, for example, oleth-2, ceteareth-2, and lauryl alcohol 3 mole ethoxylate16 mole propoxylate (ALKOMOL L 306, product of Oxiteno), and mixtures of these surfactants;

(5) trialkyl phosphates, or a mixture of trialkyl phosphates;

(6) phospholipid compounds, for example, phosphatidyl choline, phosphatidylethanolamine, and phosphatidylinositol and compositions, which include mixtures of these, for example, lecithins. Particularly preferred composition of phospholipid compounds include liquid, non-deoiled lecithins, for example, Alcolec XTRA-A, product of American Lecithin Company in Oxford, Conn.

(7) phosphate ester compounds formed from esterification of phosphoric acid with short chain polyethoxylates of C₈ to C₂₂ linear or branched, saturated or unsaturated fatty alcohols, for example, Rhodafac RP-710, Rhodafac PA32, and Lubrhophos LB400, available from Rhodia, Cranbury N.J.), and mixtures of these surfactants;

(8) Aryl alkyl carboxylic acids, for example, nonyl oxy benzoic acid, 2-(p-isobutylpheny)propionic acid (ibuprofen), 2-(6-methoxynaphthalen-2-yl)propanoic acid (naproxen), and mixtures of these compounds.

(9) Aryl alkyl alcohols, for example, nonylphenol, octylphenol, 2,2-dimethyl-3-phenylpropanol (muguet alcohol), phenyl allyl alcohol (cinnamyl alcohol), and 8-methyl-N-vanillyl-trans-6-nonenamide (capsaicin), and mixtures of these compounds.

(10) Saturated or unsaturated, linear or branched aliphatic C₈ to C₂₂ carboxylic acid functional compounds including fatty acids derived from the saponification of vegetable and animal fats and oils, for example, octanoic acid, coconut fatty acid, oleic acid, ricinoleic acid, stearic acid, and carboxylic acid terminated short chain (e.g., n=4) polymers of ricinoleic acid and mixtures of such surfactants.

(11) Saturated or unsaturated linear or branched aliphatic C₈ to C₂₂ alcohols, for example, octanol, dodecanol, myristyl alcohol, ceteryl alcohol, stearyl alcohol, isotridecyl alcohol, 3,7-dimethyl-2,6-octadien-1-ol (nerol), and so called Guerbet alcohols, for example, 2-ethyl-1-hexanol, 2-butyl-1-octanol, and 2-octyl-1-dodecanol.

(12) Saturated or unsaturated linear or branched aliphatic C₈ to C₂₂ primary and secondary amines and diamines, for example, oleyl amine, oleyl diamino propane, and cocoalkyl dimethyl amine.

### Polyethoxylated High HLB Surfactants

Suitable polyethoxylated high HLB surfactants include:

(1) polyethoxylated sorbitan esters with linear or branched long chain (greater than about 8 carbon atoms) fatty acids, for example, polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80), or a mixture of these surfactants. Particularly preferred polyethoxylated sorbitan esters include polysorbate 20 and polysorbate 80.

(2) polyethoxylate or polyethoxylate/polypropoxylate ethers with saturated or unsaturated, linear or branched aliphatic C₈ to C₂₂ alcohols, for example, poly(ethylene oxide) octyl ether, poly(ethylene oxide) dodecyl ether, poly(ethylene oxide) myristyl ether, poly(ethylene oxide) ceteryl ether, poly(ethylene oxide) stearyl ether, poly(ethylene oxide) isotridecyl ether, poly(ethylene oxide) 2-ethyl-1-hexanyl ether, poly(ethylene oxide) 2-butyl-1-octyl ether, and poly(ethylene oxide) 2-octyl-1-dodecyl ether, or a mixture of these surfactants. Particularly preferred polyethoxylate ethers with aliphatic alcohols include cetearyl alcohol 20 mole ethoxylate (ceterareth 20) and lauryl alcohol 23 mole ethoxylate (laureth-23).

(3) polyethoxylate or polyethoxylate/polypropoxylate esters with saturated or unsaturated, linear or branched aliphatic C₈ to C₂₂ carboxylic acids, for example, poly(ethylene oxide) stearate ester, poly(ethylene oxide) laurate ester, and poly(ethylene oxide) oleate ester or a mixture of these surfactants;

(4) polyethoxylated mono- and di-esters of glycerine with linear or branched long chain (greater than about 8 carbon atoms) fatty acids, for example, poly(oxyethylene) glyceryl monolaurate and poly(oxyethylene) glyceryl monostearate or a mixture of these surfactants;

(5) polyethoxylated compounds formed from the addition of ethylene oxide to linear and branched alkylphenol compounds, for example, poly(ethylene oxide) ether with nonyl phenol or octyl phenol or a mixture of these surfactants;

(6) polyethoxylated castor oils, for example, PEG-25 castor oil and PEG-40 castor oil or a mixture of these surfactants;

(7) polyethoxylated compounds formed from the addition of ethylene oxide to amide compounds formed from linear or branched long chain (greater than about 8 carbon atoms) fatty acids, for example, poly(ethylene oxide) ether with coconut acid ethanolamide or a mixture of these surfactants;

(8) polyethoxylated compounds formed from the addition of ethylene oxide to alcohol functional polysiloxanes, for example, poly(ethylene oxide) ether with methyl bis(trimethylsilyloxy)silylpropanol, or a mixture of these surfactants;

(9) EO-PO block copolymers, for example, poly(ethylene oxide)-poly(propylene oxide)-poly(ethylene oxide) block copolymers and poly(propylene oxide)-poly(ethylene oxide)-poly(propylene oxide) block copolymers, or a mixture of these surfactants;

### High HLES Non-Ethoxylated Surfactants

In addition to low HLB surfactants and polyethoxylated high HLB surfactants, compositions of the present invention can optionally include high HLB surfactants that do not contain ethylene glycol residues.

Suitable high HLB non-ethoxylated surfactants include:

(1) polyglyceryl monoesters with linear or branched long chain (greater than about 8 carbon atoms) fatty acids, for example, triglycerol monooleate, or a mixture of these surfactants;

(2) alkylated mono-, di- and oligoglycosides containing 8 to about 22 carbon atoms in the alkyl group and ethoxylated alkylated mono-, di- and oligoglycosides containing about 8 to about 22 carbon atoms in the alkyl group, for example, poly(D-glucopyranose) ether with (C₈-C₁₄) linear primary alcohols

(3) mono- and di-esters of glycerine with linear or branched long chain (greater than about 8 carbon atoms) fatty acids further esterified with short chain monocarboxylic acids, for example, glycerol monostearate lactate.

(4) amide compounds formed from linear or branched long chain (greater than about 8 carbon atoms) fatty acids, for example, acid diethanolamide and oleic acid diethanolamide (e.g., Ninol 40-CO and Ninol 201, available from Stepan Corporation, Northfield, Ill., and Hostacor DT, available from Clariant Corporation, Mount Holly, N.C.), or a mixture of these surfactants;

(5) Saturated or unsaturated, linear or branched aliphatic C₈ to C₂₂ alkyl sulfonate and sulfate compounds, for example, octanesulfonic acid, sulfuric acid ester with lauryl alcohol, sulfuric acid ester with lauryl alcohol and salts thereof, or a mixture of these surfactants;

(6) sulfonated succinic acid esters with saturated or unsaturated, linear or branched aliphatic C₈ to C₂₂ alcohols, for example, the bis(2-ethylhexyl)ester of sulfosuccinic acid and the lauryl poly(ethylene oxide) ester of sulfosuccinic acid, or a mixture of these surfactants;

(7) sulfuric acid esters of linear or branched long chain (greater than about 8 carbon atoms) alcohol ethoxylates, alcohol propoxylates, alcohol ethoxylate propoxylates and ethoxylated linear and branched alkylphenol compounds and salts thereof, for example, sodium dodecylpoly(oxyethylene) sulfonate and sodium poly(oxyethylene) octyl phenyl ether sulfonate, or a mixture of these surfactants;

(8) sulfonates of benzene, cumene, toluene and alkyl substituted aromatic compounds and salts thereof, for example, dodecyl benzene sulfonic acid, or a mixture of these surfactants;

(9) carboxylates of alcohol ethoxylates, alcohol propoxylates, alcohol ethoxylate propoxylates and ethoxylated linear and branched alkylphenol compounds and salts thereof, for example, poly(ethylene oxide)tridecyl alcohol ether carboxylic acid and sodium poly(ethylene oxide) lauryl ether carboxylate, or a mixture of these surfactants;

(10) long chain (greater than about 8 carbon atoms) acyl amino acids, for example, acyl glutamates, acyl peptides, acyl sarcosinates, acyl taurates, salts thereof, and mixtures of these surfactants;

(11) Saturated or unsaturated, linear or branched aliphatic C₈ to C₂₂ alkyl amido propyl (dimethyl ammonio)acetate compounds, for example, lauramidopropyl betaine and stearamidopropyl betaine, and mixtures of these surfactants.

(12) Sophorolipids, which consist of a hydrophobic fatty acid tail of a hydroxylated 16 or 18 carbon atom fatty acid, which is β-glycosidically linked to a hydrophilic sophorose head, including free acid (open) and internally esterified (lactonic) forms and acetylated forms (acetylated on the 6′- and/or 6″-positions). Sophorolipids useful in the practice of this invention include product mixtures produced by yeasts, for example. Candida bombicola, Candida apicola, Starmerella bombicola, and Candida sp. NRRL Y-2720 (as identified by Price et al., Carbohydrate Research 348 (2012) 33-41) and chemically modified product mixtures.

(13) Rhamnolipids including mono-rhamnolipids, which consist of one or two 3-(hydroxyalkanoyloxy) alkanoic acid tails and a single rhamnose head and di-rhamnolipids, which consist of one or two 3-(hydroxyalkanoyloxy) alkanoic acid tails and two rhamnose heads, including mixtures of compounds produced by Pseudomonas and Burkholderia bacterial species, for example, Pseudomonas aeruginosa and Burkholderia plantarii.

### Cationic Surfactants

Compositions of the present invention optionally include cationic surfactants. Suitable cationic surfactants include:

(1) fatty alkyl primary and secondary amine and heterocyclic ring functional compounds, for example, oleyl amine, oleyl diaminopropane, alkenyl and aryl alkyl substituted azlactone ring compounds, and alkenyl substituted imidazole ring compounds, for example, oleyl hydroxyethyl imidazoline;

(2) fatty alkyl tertiary amine compounds, for example, lauryl dimethyl amine and cocoalkyl dimethyl amine;

(3) quaternary ammonium salts, for example, didecyl dimethyl ammonium chloride and benzalkonium chloride; and mixtures of these surfactants

### Oil/Surfactant Ratio

The physicochemical properties of microemulsion lipid bilayers are critical to the formation of small unilamellar liposomes depends upon the properties of the oil and the amount of oil present.

The phase behavior of mixtures of oil, water and surfactants is conveniently described using phase diagrams. A relevant phase diagram for compositions that give lamellar phase microemulsions is the pseudo binary phase diagram with a surfactant endpoint and water plus oil endpoint on the horizontal axis and temperature on the vertical axis. In such pseudo binary phase diagrams for mixtures containing oil, water, and ethoxylated surfactants, phase boundaries frequently take a fish shaped appearance with the head oriented in the direction of the oil plus water endpoint and the tail oriented towards the surfactant endpoint. The body of the fish and the area surrounding the fish represent three and two phase regions, respectively, and the tail represents a single phase microemulsion region, the width of which expands in the temperature dimension as the amount of surfactant in the system increases. Preferable precursors for oil swollen unilamellar liposomes are single phase lamellar microemulsions which occur in the tail section of pseudo binary oil-water-surfactant diagrams. Although the tail in oil-water-surfactant pseudo binary phase diagrams may be divided into an inner lamellar liquid crystal region which exhibits depressed electrical conductivity and decreased transparency plus a surrounding isotropic region (F. Schambil et al., “Interfacial and colloidal properties of cosmetic emulsions containing fatty alcohol and fatty alcohol polyglycol ethers,” Progr Colloid & Polymer Sol, 73:37-47 (1987); P. Izquierdo et al.,“Phase Behavior and Nano-emulsion Formation by the Phase Inversion Temperature Method,” Langmuir, 2004, 20, 6594-6598), the presence of lamellae critical for formation of small unilamellar liposomes can be discerned throughout the entire single phase tail region by including macroscopic observation through crossed polarizing films along with the conventional microscopic observation. The effect of the ratio of oil to surfactants on the extent of lamellar character in a composition of oil, water, and surfactant can then be gauged in terms of the degree to which conductivity is depressed in the inner lamellar liquid crystal region of the tail, the temperature range in which conductivity is depressed, and the temperature range that the composition exhibits either macroscopic or microscopic birefringence.

As demonstrated by Example 5, the extent of lamellar character in microemulsions increases and then decreases as the ratio of oil to surfactants increases. A mixture of surfactants and water with no oil shows a monotonic increase in conductivity as it cools, with no intermediate transparent single phase region and a minimum amount of oil is required for lamellae and microemulsion formation. The presence of lamellae can be discerned as a reduction in conductivity in the form of a very broad negative peak and temperature regions in which the composition is transparent and shows birefringence. Further addition of oil causes an increase in the temperature range within which the composition is a single phase microemulsion and the development of an intermediate inner lamellar liquid crystal region (inner less transparent tail region of a pseudo ternary phase diagram). Beyond an optimal amount of oil, lamellar character of the composition decreases until once again no microemulsion is observed and conductivity increases monotonically when the composition cools. For the system of isopropyl myristate, polysorbate-80 and lecithin, evidence for lamellar character was observed at oil to surfactant ratios ranging between 0.12:1 and 4.5:1, with maximum formation at ratios of oil to surfactant between about 0.24:1 and about 2.5:1. Microemulsions were observed at oil to surfactant ratios ranging between 0.12:1 and 3.5:1. In preferred compositions for the preparation of liposomes from lamellar phase microemulsions, the ratio of oil to surfactant is between 0.12:1 to 3.5:1, between 0.24:1 to 2.5:1, and between 0.48:1 to 2.0:1.

### Additional Components

The niosome compositions can contain additional components if desired. For example, the compositions can contain adjuvants, for example, antimicrobial agents, colorants. UV absorbers, aroma oils, viscosity modifiers, or antioxidants. The amounts and types of such additional components will be apparent to those skilled in the art.

### Methods

In general, the niosome compositions are prepared by a first step of combining a high K_{ow} pharmacologically active compound, a water immiscible oil, a low HLB surfactant, a polyethoxylated high HLB surfactant, water, and additional components if desired, mixing with low to moderate shear, and heating the mixture to a temperature where a microemulsion phase exists. In a second step, the microemulsion is rapidly cooled or rapidly cooled and diluted by water or an aqueous composition. In preferred embodiments, the water used for dilution includes additional components, particularly additional components that are unstable at elevated temperatures.

Preferably, the microemulsion is cooled or diluted and cooled to a temperature less than about 40° C. The rate of cooling of the high temperature microemulsion phase is preferably greater than about 1° C. per minute, greater than about 5° C. per minute, greater than about 20° C. per minute, and greater than about 40° C. per minute.

In preferred embodiments, a precursor microemulsion is prepared in a separate vessel and pumped to a second vessel containing water or an aqueous composition.

In preferred embodiments, a precursor coarse emulsion is pumped through a heat exchanger to raise the temperature to a temperature at which a microemulsion exists and then to a second vessel containing water or an aqueous composition.

In preferred embodiments, a precursor microemulsion is prepared in a vessel and pumped through a cooling heat exchanger to provide a niosome product composition that is undiluted from the microemulsion. Optionally, a second fluid stream may be joined with the flow of microemulsion just before or after the heat exchanger to provide additional components to the formulation.

In preferred embodiments, a precursor coarse emulsion is pumped through a heat exchanger to raise the temperature to a temperature at which a microemulsion exists and then through a second cooling heat exchanger to provide a niosome product composition that is undiluted from the precursor coarse emulsion. Optionally, a second fluid stream may be joined with the flow of microemulsion just before or after the cooling heat exchanger to provide additional components to the formulation.

In preferred embodiments, a precursor coarse emulsion is pumped through a microwave heating zone to raise the temperature to a temperature at which a microemulsion exists and then through a cooling heal exchanger to provide a niosome product composition that is undiluted, from the precursor coarse emulsion. Optionally, a second fluid stream may be joined with the flow of microemulsion just before or after the heat exchanger to provide additional components to the formulation.

The composition for the topical prevention and treatment of a disorder in humans may be applied in a single administration or in multiple administrations. The compositions are topically applied for at least one day, at least two days, at least three days, at least four days, at least 5 days, once a week, at least twice a week, at least once a day, at least twice a day, multiple times daily, multiple times weekly, biweekly, at least once a month, or any combination thereof.

The composition for the topical prevention and treatment of disorder in humans may be topically applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years.

Preferably, the composition is applied topically to the involved area until it has healed. The composition is preferably administered six to eight times a day for from one day to a week or more until healing occurs.

### EXAMPLES

Unless otherwise indicated, all numbers expressing quantities of ingredients, properties, for example, molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

In the Examples, the following analytical methods were used. Electrical Conductivity was measured using a Thermo Scientific Orion 3-star Conductivity Meter Model 1114000 with a 013005MD 4-cell conductivity cell electrode. Particle size was determined by dynamic laser light scattering using a Beckman Coulter Delsa Nano C Particle Analyzer. Turbidity was measured using a Hach 2100 Turbidimeter. Specific turbidity values of samples were calculated as the least squares slope of turbidity vs non-water concentration in the range of 0 and 600 ppm non-water. Cryo-TEM samples were prepared by applying a 3 μL drop of sample suspension onto cleaned holey carbon films on 400-mesh copper grids, blotting away with filter paper, and immediately vitrifying in liquid ethane. Grids were stored under liquid nitrogen until transferred to the electron microscope for imaging. Vitrified samples were transferred into the electron microscope using a cryostage that maintains the samples at a temperature below −170° C. Electron microscopy was performed using an FEI Tecnai T12 electron microscope operating at 120 keV equipped with an FEI Eagle 4 k×4 k CCD camera. Images were acquired at a nominal under focus of −5 μm to −2 μm and electron doses of about 10-30 electrons/Å².

### Example 1

### Preparation of Micro Emulsions Including Fractionated Coconut Oil, Polysorbate-80, Lecithin and Ibuprofen

Fractionated coconut oil (60 grams of Lotioncrafter FCO, available from Lotioncrafter, Eastsound, Wash.), polysorbate 80 (12 grams of Lumisorb PSMO-2OK, available from Lambent Technologies, Gurnee, Ill.), lecithin (12 grams of Alcolec XTRA-A, available from American Lecithin, Oxford, Conn.) and distilled water (61 grams) were weighed into a 250 mL beaker. The contents were heated to about 96° C. and allowed to cool while stirring with a magnetic stir bar and logging the temperature and conductivity. The mixture started out as very hazy, becoming more transparent at about 88° C. and began to become more opaque at about 81° C. It remained opaque as it cooled to about 66° C.

After cooling, ibuprofen (1.5 grams, purified from Walgreen's brand of 200 mg generic tablets, available from the Walgreen Company, Deerfield, Ill., by extracting with about 91% isopropanollwater and recrystallized from isopropanol/water) was added and water that evaporated during heating and cooling was replaced. The heating and cooling process repeated from about 92° C. to about 60° C. The mixture was fairly transparent in the region of about 76° C. to about 89° C. After cooling and replacing evaporated water, an additional 1.5 grams of ibuprofen was added (total 3 grams ibuprofen) and the mixture cooled from about 94° C. to about 52° C. In this case, the mixture was fairly transparent between about 72° C. to about 80° C. The cycle of replacing water and adding more ibuprofen was continued with addition of 1.5 grams more ibuprofen (total 4.5 grams ibuprofen) and the mixture cooled from about 82° C. to about 54° C. In this case, the mixture was fairly transparent between about 67° C. to about 71° C. Addition of 1.5 more grams ibuprofen (for a total of 6 grams) gave a mixture with a transparent region between about 62° C. and about 64° C., and subsequent addition of 4 grams more ibuprofen gave a mixture with no transparent region between about 31° C. and about 94° C.

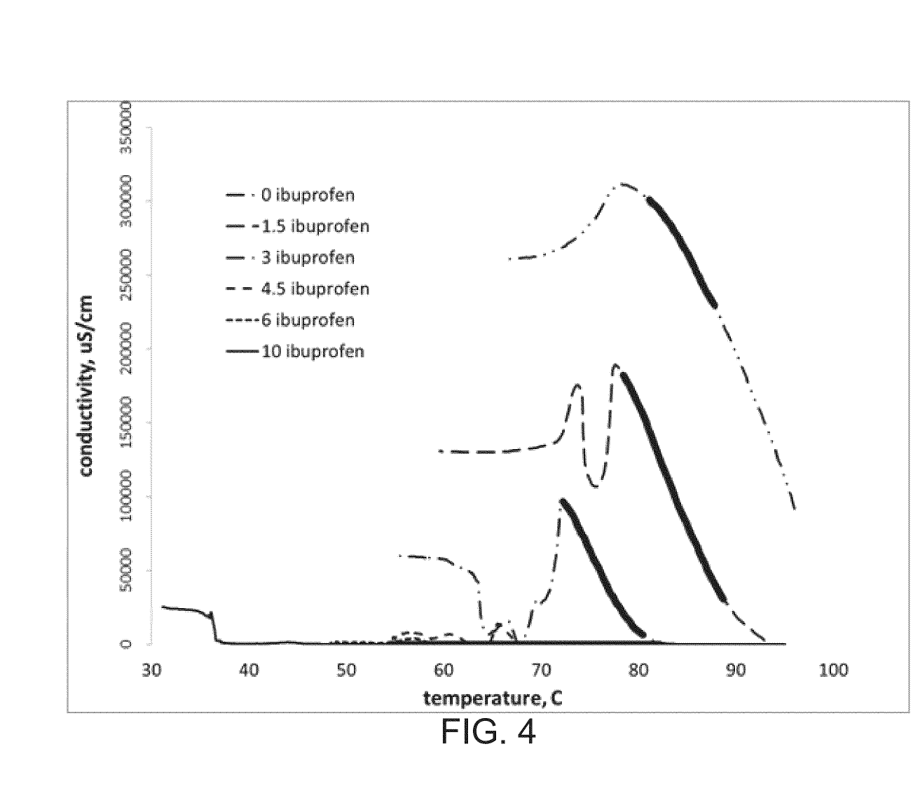

Plots of conductivity (uS/cm) vs. temperature (° C.) for the heating and cooling processes are shown in FIG. 4. The regions where each composition was transparent or hazy transparent is indicated by wide lines in the plots. The low conductivity of samples with greater than 4 grams of ibuprofen (remaining under 20 mS/cm compared to over 300 mS/cm for the composition without ibuprofen) is indicative of the presence of extensive lamellar structures.

This Example demonstrates that the ibuprofen Increases the extent and the ridigdity of lamellar microemulsion phases, as indicated by the diminishment of electrical conductivity.

### Example 2

### Preparation of Micro Emulsions Including Fractionated Coconut Oil, Polysorbate-80, Lecithin and Naproxen

Fractionated coconut oil (60.0 grams of Lotioncrafter, available from Lotioncrafter, Eastsound, Wash.), polysorbate 80 (12.0 grams of Lumisorb PSMO-20K, available from Lambent Technologies, Gurnee, Ill.), lecithin (12.1 grams of Alcolec XTRA-A, available from American Lecithin, Oxford, Conn.) and distilled water (60.4 grams) were weighed into a 250 mL beaker. The contents were heated to about 98° C. and allowed to cool while stirring with a magnetic stir bar and logging the temperature and conductivity. The mixture started out as very hazy, becoming more transparent at about 88° C. and began to become more opaque at about 81° C. It remained opaque as it cooled to about 66° C.

After cooling, naproxen (1.5 grams, prepared from Walgreen's brand of 220 mg generic naproxen sodium tablets, available from the Walgreen Company, Deerfield, Ill., by extracting with distilled water, acidifying with one equivalent of hydrochloric acid, and recrystallizing from isopropanol/water) was added and water that evaporated during heating and cooling was replaced. The heating and cooling process was repeated from about 95° C. to about 60° C. The mixture was fairly transparent and isotropic in the region of about 78° C. to about 93° C. After cooling and replacing evaporated water, an additional 1.5 grams of naproxen was added (total 3.0 grams naproxen) and the mixture cooled from about 94° C. to about 51° C. In this case, the mixture was fairly transparent and isotropic between about 68° C. to about 86° C. The cycle of replacing water and adding more naproxen was continued with addition of 1.5 grams more naproxen to give total naproxen equal to 4.6 grams, 6.1 grams, 7.6 grams, and 9.2 grams. For each amount of naproxen added, a temperature range existed where the composition was significantly more transparent and isotropic, indicating a microemulsion.

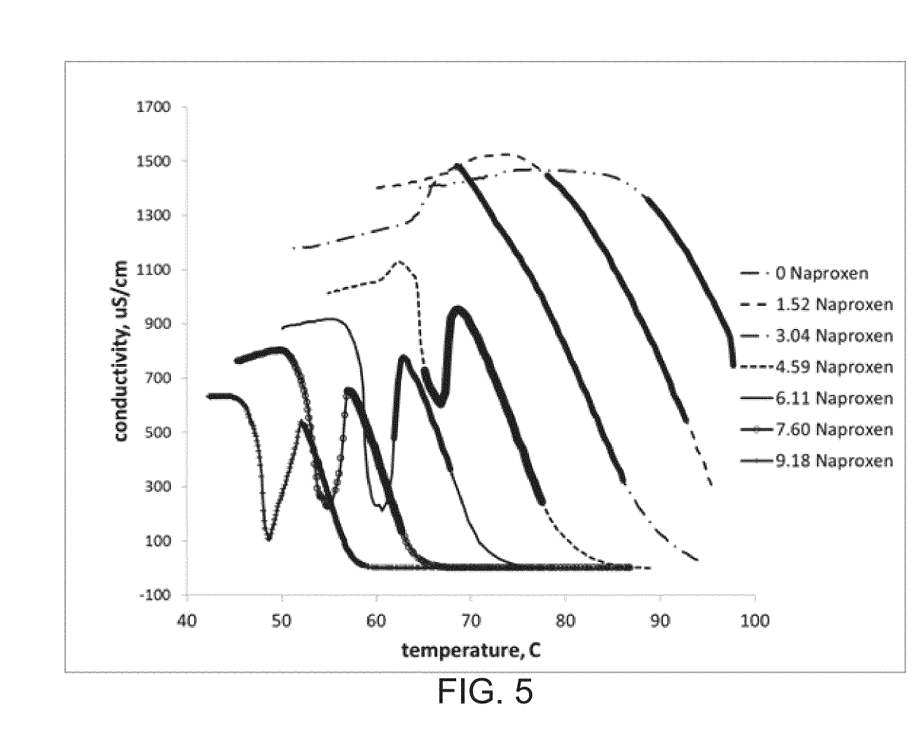

Plots of conductivity (uS/cm) vs. temperature (° C.) for the heating and cooling processes are shown in FIG. 5. The region where each composition was transparent or hazy transparent is indicated by wide solid lines in the plots. The diminishment of conductivity in samples with increasing amounts of naproxen is indicative of increasing extensiveness and/or rigidity of lamellae in the microemulsions.

This Example demonstrates that the naproxen increases the extent and or the rigidity of lamellar microemulsion phases, as indicated by the diminishment of electrical conductivity. The development of lamellar character in microemulsions attributable to naproxen addition is less than that of ibuprofen addition on a per mass basis.

### Example 3

### Preparation of Micro Emulsions Including Isopropyl Myristate, Polysorbate-80, Lecithin and Aspirin

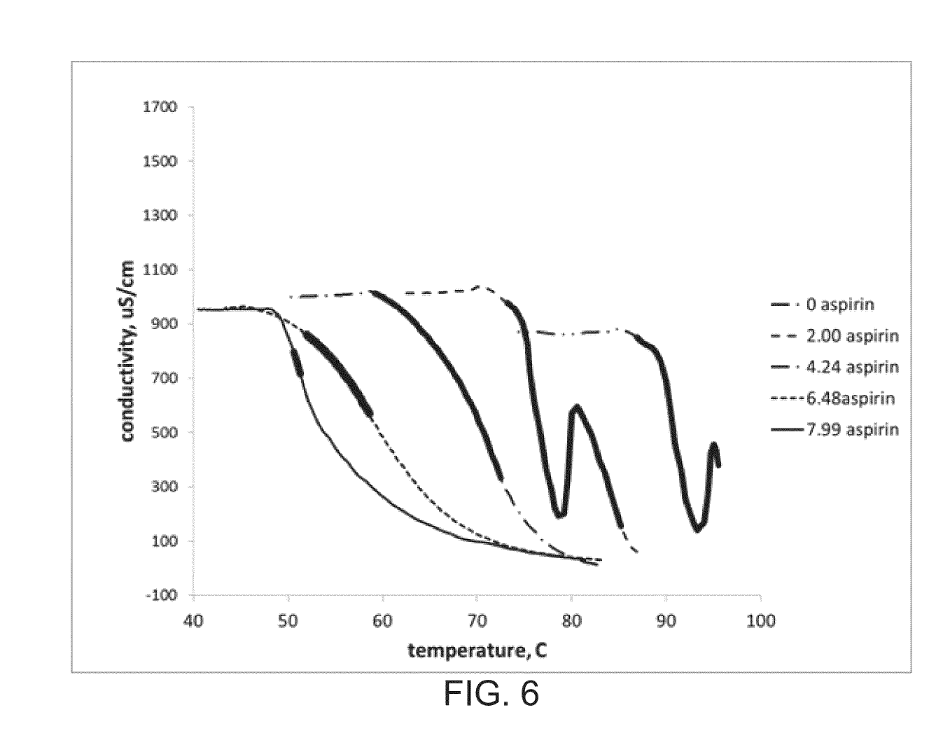

Isopropyl myristate (60.0 grams, available from Lotioncrafter, Eastsound, Wash.), polysorbate 80 (12.0 grams of Lumulse PSMO-20K, available from Lambent Technologies, Gurnee, Ill.), lecithin (6.0 grams Alcolec XTRA-A, product of American Lecithin, Oxford, Conn.) and water (60.1 grams) were heated to about 94° C. and allowed to cool while measuring temperature and conductivity. The mixture was a microemulsion above about 87° C. as evidenced by exhibiting an isotropic and moderately transparent appearance. Subsequently, the temperature scan was repeated four times with addition of aspirin (o-acetyl salicylic acid, purified from Walgreen's brand 325 mg tablets, available from the Walgreen Company, Deerfield, Ill., by extraction with methanol, filtration, and recrystallization from methanol/water). In each subsequent conductivity versus temperature scan, the amount of aspirin was increased by about 2 grams for a total of 2.0, 4.2, 6.5, and 8.0 grams of aspirin, respectively. Plots of conductivity (uS/cm) vs. temperature (° C.) for the compositions are shown in FIG. 6. The region where each composition was isotropic and transparent or hazy transparent is indicated by wide solid lines in the plots. As the amount of aspirin in the composition increased the local minimum in conductivity originally at about 93° C. shifted to about 79° C. and disappeared. The loss of the negative peak (local minimum in conductivity) is indicative of the diminishment of the extensiveness and/or rigidity of lamellae in microemulsions.

This Example demonstrates that the slightly hydrophobic aspirin (pK_{ow}=1.25) decreases the extent and/or the rigidity of lamellae in microemulsion phases including isopropyl myristate, polysorbate-80, lecithin and water, shifts the phase inversion to lower temperatures, and diminishes the temperature range in which the composition is a microemulsion.

### Example 4

### Preparation of Micro Emulsions Including Isopropyl Myristate, Polysorbate-80, Lecithin and Acetaminophen

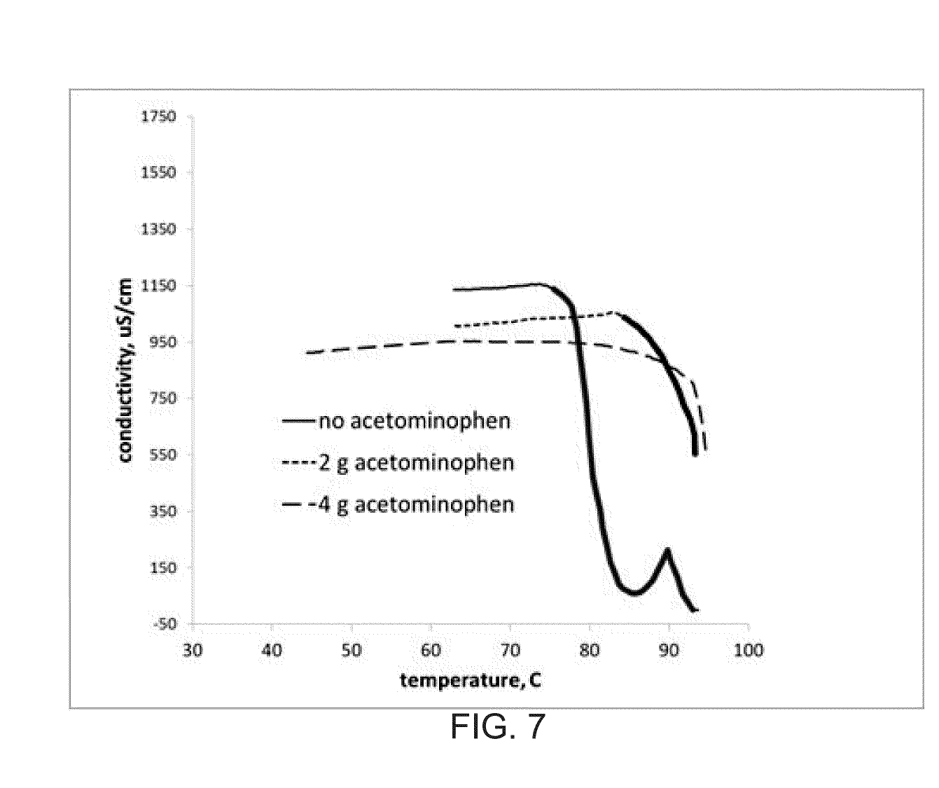

Conductivity (uS/cm) vs. temperature (° C.) was measured for a composition of 12.0 grams polysorbate-80, 9.3 grams Alcolec XTRA-A lecithin, 59.0 grams isopropyl myristate, and 60.0 grams water. The composition was transparent and isotropic between about 76° C. and about 93° C. Subsequently, water that evaporated out was replaced and acetaminophen (2.0 grams N-acetyl-p-aminophenol, purified from Walgreen's brand 200 mg gel caps, available from the Walgreen Company, Deerfield, Ill., by extracting with methanol, filtration, and recrystallization from methanol/water) was added and conductivity (uS/cm) vs. temperature (° C.) measured again. This composition was transparent and isotropic between about 84° C. and about 93° C. In a final experiment, water that evaporated was replaced and an additional 2.0 grams of acetaminophen added. The conductivity was measured and the sample appearance observed between about 44° C. and about 93° C. The sample was not transparent in this temperature range. Plots of conductivity (uS/cm) vs. temperature (° C.) are shown in FIG. 7. This Example demonstrates that relatively hydrophilic acetaminophen (with pK_{ow}=0.5) lessens the lamellar character of a composition of polysorbate-80, lecithin, isopropyl myristate, and water and inhibits microemulsion formation.

### Example 5

### Preparation of Drug-Free Micro Emulsions Including Isopropyl Myristate, Polysorbate-80 and Lecithin

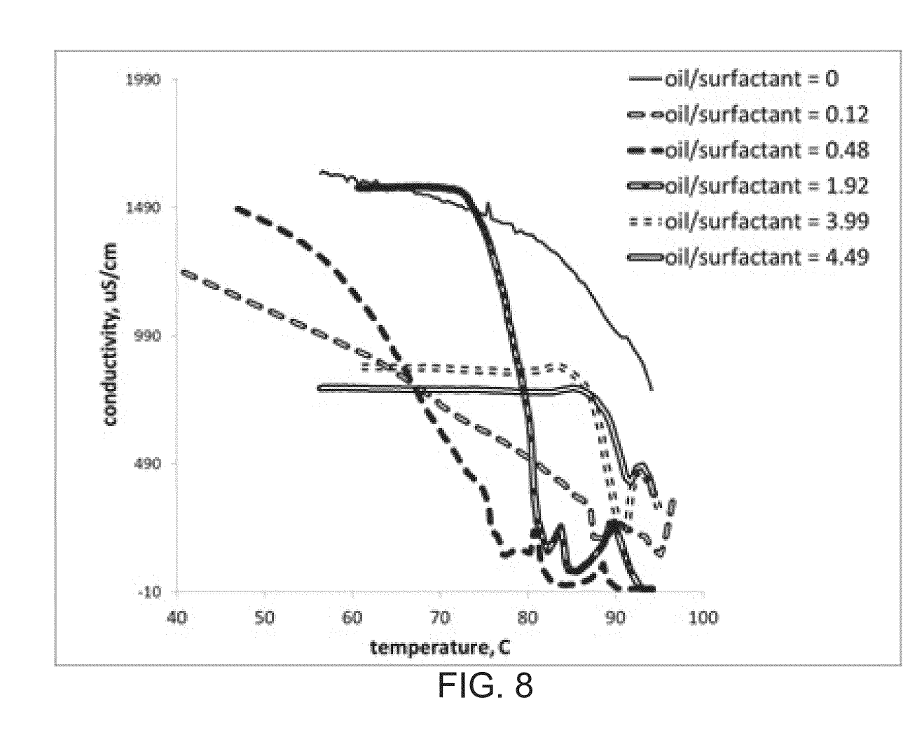

Polysorbate-80 (Lotioncrafter, 18.0 grams), lecithin (American Lecithin Alcolec XTRA-A, 13.5 grams), and 90.2 grams of distilled water (ratio of isopropyl myristate/surfactant=0) were mixed and heated and the appearance and conductivity measured as the sample cooled. At high temperatures (about 96° C.), the sample was translucent and hazy and appeared isotropic. The mixture was viscous, and there was a small amount of foam on the surface. The composition showed a monotonic increase in conductivity as temperature dropped, with no local maxima or minima. After replacing water, 3.8 grams of isopropyl myristate (Lotioncrafter IPM, ratio of IPM:surfactant=0.12:1) was added and conductivity (uS/cm) vs. temperature (° C.) again evaluated. In this case, there was a substantial depression of conductivity at all temperatures evident as a very broad negative peak centered at about 92° C. with two local minima at about 89° C. and about 95° C. The mixture was isotropic and transparent between about 86° C. and about 96° C., and when viewed through crossed polarizing filters, was birefringent. After replacing water, 3.8 grams of isopropyl myristate was added (7.6 grams total IPM, ratio of IPM:surfactant=0.24:1) and conductivity (uS/cm) vs. temperature (° C.) again evaluated. In this case, there was a substantial depression of conductivity evident as a very broad negative peak with local minimum at about 87° C. and the mixture was isotropic and transparent to the unaided eye and birefringent above about 91° C. and also from about 78° C. to about 87° C. In subsequent experiments, additional isopropyl myristate was to bring the ratio of oil to surfactant in the system sequentially to 0.48:1, 0.97:1, 1.92:1, 2.49:1, 2.99:1, 3.49:1, 3.99:1, and 4.49:1. At each ratio of oil to surfactant, conductivity was measured as the sample cooled and the temperature range that the sample was transparent and birefringent noted. Results of the observations are shown in Table 3 and a graph showing conductivity (uS/cm) vs. temperature (° C.) for compositions with ratios of oil to surfactant=0, 0.12, 0.48, 1.92, 3.99, and 4.49 are shown in FIG. 8.

This Example demonstrates that addition of isopropyl myristate to a mixture of lecithin, polysorbate-80 and water causes the formation of lamellar structures over a wide range of compositions (oil to surfactant ratios of 0.12:1 to 4.5:1) as evidenced by depression of electrical conductivity. The extent of conductivity suppression is greatest for small additions of oil and decreases with increasing amounts of oil and the microemulsion temperature range first increases then decreases with increasing amounts of oil with maximum formation at ratios of oil to surfactant between about 0.24:1 and about 2.5:1.

TABLE 3

| oil/ | | |
|---|---|---|
| surfactant | transparent | birefringent |
| ratio | temperature range, C. | temperature range, C. |
| 0 | — | — |
| 0.12 | 86-96 | 87-89 |
| 0.24 | 75-87, 91-87 | 75-87, 91-87 |
| 0.48 | 78-82, 89-90 | 78-82, 89-90 |
| 0.97 | 79-81, 87-90 | 79-81, 87-90 |
| 1.92 | 85-87, 91-93 | not measured |
| 2.49 | 75-86, 90-93 | 75-79, 90-91 |
| 2.99 | 81-94 | 81-82, 91-93 |
| 3.49 | 90-92 | — |
| 3.99 | — | — |
| 4.49 | — | — |

### Example 6

### Preparation of Micro Emulsions Including Ibuprofen, Fractionated Coconut Oil, Laureth-23 and Lecithin

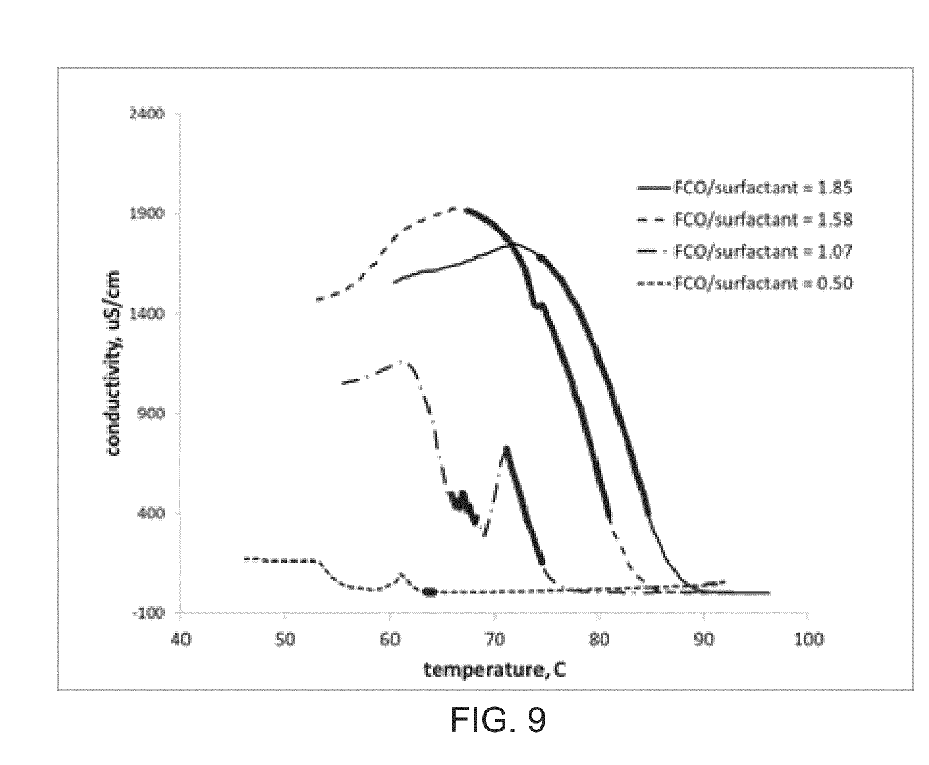

Ibuprofen (available from the Walgreen Company, Deerfield, Ill., crystallized from isopropanol/water, 9.0 grams), fractionated coconut oil (50.0 grams, Lotioncrafter FCO), laureth-23 (12.0 grams Lotioncrafter), lecithin (15.02 grams, American Lecithin Alcolec XTRA-A), and 60.2 grams of water (ratio of FCO/surfactant=1.39/1 including ibuprofen) were heated and the appearance and conductivity measured as the sample cooled from about 96° C. to about 61° C. The conductivity (uS/cm) vs. temperature (° C.) is shown in FIG. 9. In FIG. 9, temperature regions where compositions are isotropic and either transparent or translucent are indicated by thick continuous lines. The composition was isotropic and transparent in the temperature range from about 74° C. to about 85° C. After the first heating and cooling cycle, water that evaporated was replaced and 2.1 grams laureth-23, 2.5 grams lecithin, 1.5 grams ibuprofen, and 10.2 grams water were added (ratio of FCO surfactant 1.19/1). The heating and cooling was repeated, and the sample was isotropic and transparent between about 67° C. and about 81° C. After the second heating and cooling cycle, an additional 7.0 grams laureth-23, 8.8 grams lecithin, 5.3 grams ibuprofen, and 35 grams water were added (ratio of FCO surfactant=0.80), and the conductivity scan repeated. In this case the composition was isotropic and transparent between about 66° C. and about 69° C. and between about 71° C. and about 75° C. To 66.0 grams of the composition, an additional 7.1 grams laureth-23, 9.0 grams lecithin, 5.3 grams ibuprofen, and 35.0 grams water were added (ratio of FCO surfactant=0.38), in which case the composition was isotropic and transparent between about 63° C. and about 64° C. Throughout the heating and cooling cycle, the sample remained quite viscous and was difficult to stir. This Example demonstrates that the lamellar character increases as the ratio of oil to surfactant decreases for a composition of ibuprofen, fractionated coconut oil, laureth-23, lecithin, and water.

### Example 7

### Phase Behavior of Oil, Water, and Two Dissimilar Surfactants with Similar Values of HLB

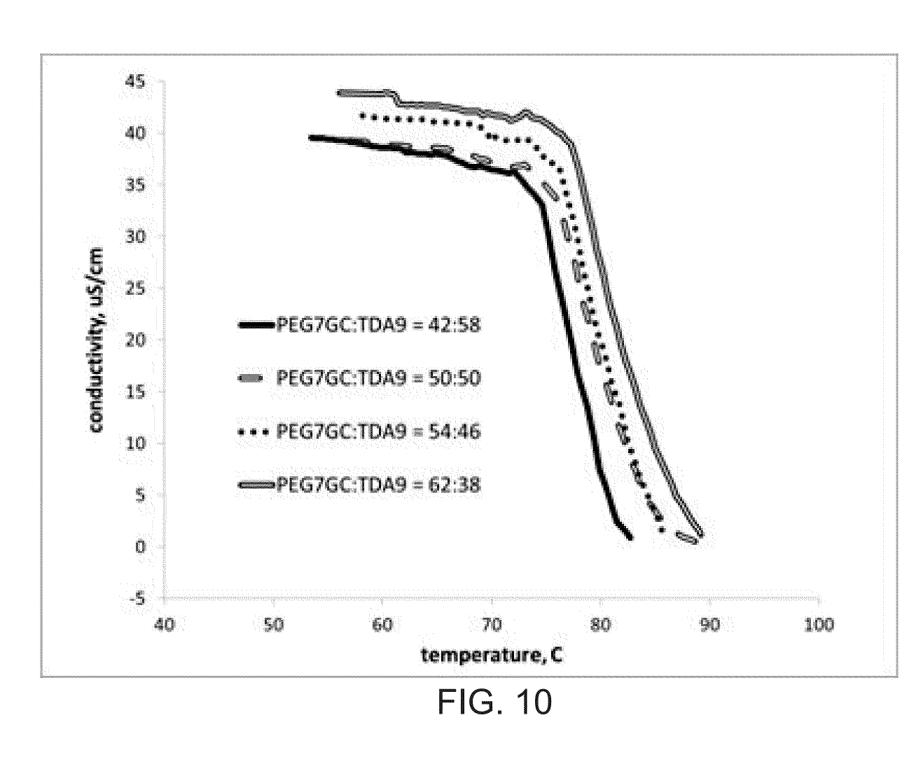

Conductivity (uS/cm) vs. temperature (° C.) was measured for a composition containing 7.91 grams PEG-7-glyceryl cocoate (PEG7GC, available from Making Cosmetics, Snoqualmie, Wash.), 11.1 grams isotridecyl 9 mole ethoxylate (Novel TDA-9, product of Sasol North America, Houston, Tex.), 47.50 grams isopropyl myristate, and 47.61 grams water. The HLB values for PEG-7-glyceryl cocoate and Novel TDA-9 were calculated using a proprietary algorithm by Molecular Modeling Pro software, version 5.22, commercialized by Norgwyn Montgomery Software Inc. ©2003 as 14.3 and 13.8, respectively. The weight ratio of PEG-7-glyceryl cocoate to TDA-9 was 42:58, the weight average HLB value for the mixture was 14.0, and the HLB weight mean square deviation (WMSD_{HLB} was 0). As the mixture cooled from about 83° C. to about 54° C., the conductivity remained low, then increased monotonically and abruptly at about 77° C. as shown in FIG. 10. In this temperature range, the composition remained opaque and was never transparent and isotropic. Subsequently, additional TDA-9 surfactant, water, and isopropyl myristate were added to the sample to increase the weight ratio of PEG-7-glyceryl cocoate to Novel TDA-9 to 50:50, 54:46, and 62:38 while maintaining the weight ratio of isopropyl myristate to total surfactant equal to 2.5:1 and the weight ratio of isopropyl myristate to water equal to 1:1. As the weight ratio of PEG-7-glyceryl cocoate to Novel TDA-9 increased, the weight average HLB value increased slightly from 14.0 at 42:58 to 14.1 at 62:38, WMSD_{HLB} remains at 0 and the phase inversion temperature also increased slightly from about 77° C. to about 82° C. as shown in FIG. 10. None of the compositions gave transparent isotropic microemulsions. This Example demonstrates that the phase inversion of compositions of oil, water, and mixtures of two dissimilar surfactants with similar HLB values occurs without intermediate microemulsion phases or evidence of lamellar phases.

### Example 8

### Phase Behavior of Oil, Water, and Three Dissimilar Surfactants with Low HLB Polydispersity

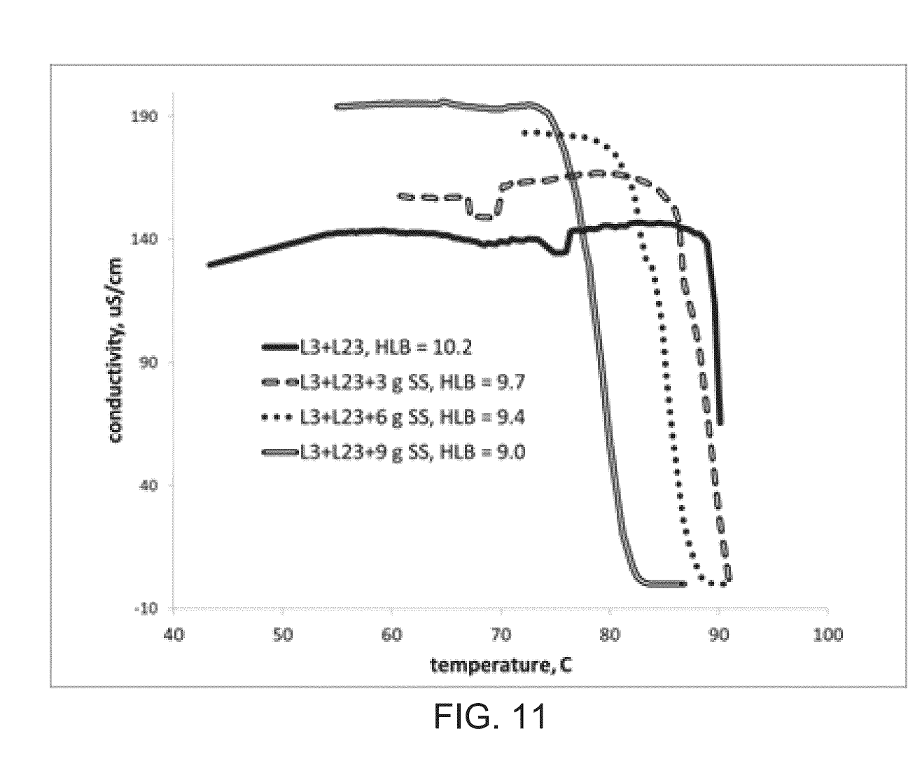

A mixture of 19.84 grams laureth-3 (available from Making Cosmetics, Snoqualmie, Wash.), 5.66 grams laureth-23 (Lotioncrafter), 51.0 grams isopropyl myristate, and 51.00 grams water was heated to about 90° C. and allowed to cool to about 43° C. while recording the conductivity and temperature. The HLB values for laureth-3 and laureth-23 were calculated using Molecular Modeling Pro software, version 5.22, as 9.2 and 17.9, respectively. The weight ratio of laureth-3 to laureth-23 was 78:22, the weight average HLB value for the mixture was 11.1, and the HLB weight mean square deviation (WMSD_{HLB}) was 1.17. The composition was not isotropic or transparent or translucent in the temperature range from about 43° C. to about 90° C. In three subsequent experiments, sorbitan stearate (Lotioncrafter, HLB=6.7 as calculated using Molecular Modeling Pro software, version 5.22) was added to give total sorbitan stearate=3.02 grams, 6.04 g, and 9.33 grams and at each step conductivity versus temperature was measured. In each case there was a rapid monotonic increase in conductivity as temperature decreased, and no composition exhibited an isotropic translucent or transparent region. A plot of conductivity (uS/cm) vs. temperature (° C.) for the four compositions is shown in FIG. 11. The weight average HLB and the WMSD in HLB calculated as described above were calculated for each composition and the values are shown in Table 4 below. This Example demonstrates that for this composition, the maximum polydispersity as WMSD_{HLB}=1.35 is not effective to promote sufficient lamellar character so as to give an isotropic microemulsion.

TABLE 4

| | sorbitan stearate (grams) | | | |
|---|---|---|---|---|
| | 0.00 | 3.02 | 6.04 | 9.33 |
| weight average HLB | 11.1 | 10.7 | 10.3 | 9.9 |
| HLB weight mean square deviation, WMSD | 1.17 | 1.27 | 1.32 | 1.35 |

### Example 9

### Phase Behavior of Oil, Water, and Three Dissimilar Surfactants with Moderate HLB Polydispersity

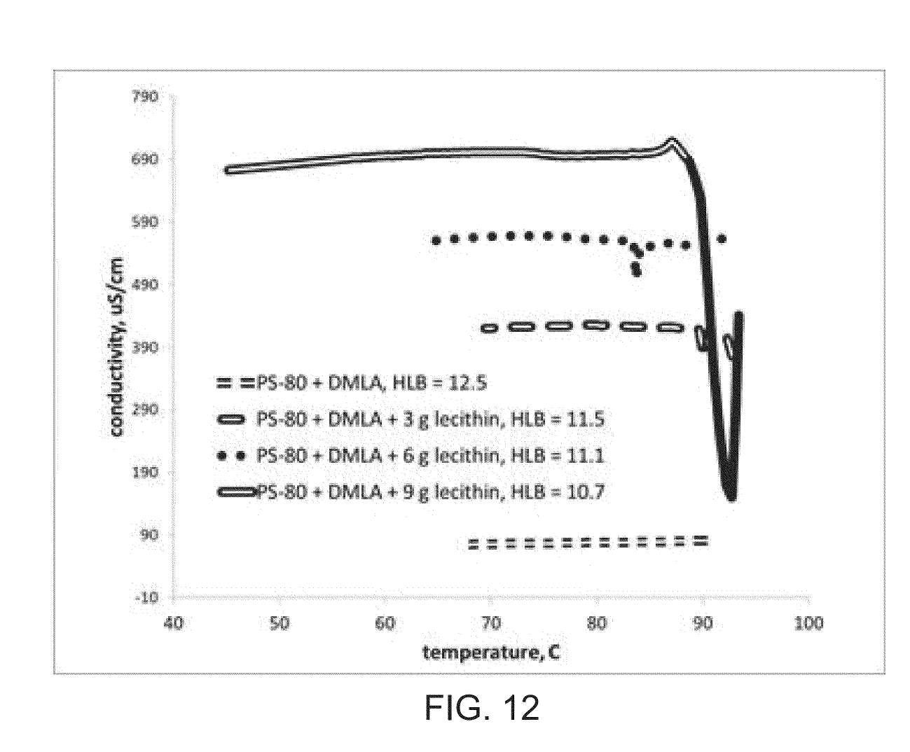

A mixture of 11.99 grams polysorbate-80 (Lotioncrafter), 18.08 grams dimethyllauryl amine (DMLA), (Dimethyl C10-C16 fatty amines (AT-1214LFFA), product of Proctor and Gamble Chemicals), 50.84 grams isopropyl myristate, and 50.13 grams water was heated to about 90° C. and allowed to cool to about 67° C. while measuring conductivity. The HLB value for polysorbate-80 was 16.5 as calculated using Molecular Modeling Pro software, version 5.22 and the HLB value for DMLA was 9.8 (taken from the manufacturer's technical data sheet). The weight ratio of polysorbate-80 to DMLA was 40:60, the weight average HLB value for the mixture was 12.5, and the HLB weight mean square deviation (WMSDHLB) was 0.86. In this experiment, the conductivity remained nearly constant between about 75 and 80 μS/cm and the composition remained opaque. In three subsequent experiments, conductivity versus temperature was recorded for the same composition but with incremental addition of lecithin (Alcolec XTRA-A, product of American Lecithin, Oxford, Conn.) to give total lecithin equal to 3.09 grams, 4.60 grams, and 6.09 grams. The HLB value of Alcolec XTRA-A lecithin according to the manufacturer is 2. Weight average HLB and WMSD_{HLB} values for the four compositions are shown in Table 5. With 3.09 grams or 4.60 grams of lecithin, the samples exhibited nearly constant conductivity as a function of temperature while cooling and remained opaque. With a total of 6.09 grams of lecithin, the sample showed a large negative peak in conductivity at about 92° C. and isotropic transparency between about 88° C. and about 93° C., indicating lamella and microemulsion formation. Plots of conductivity (uS/cm) vs. temperature (° C.) for the compositions are shown in FIG. 12. The temperature region in which compositions were isotropic microemulsions is indicated by plotting with a filled black line. This Example demonstrates that the minimal compositions with minimal polydispersity of the surfactant HLB values do not show lamellar character or microemulsions, and that increasing the polydispersity calculated as WMSD_{HLB} to values greater than about 2 is sufficient to promote lamellar character and microemulsion formation for a composition with isopropyl myristate, polysorbate-80, dimethyl lauryl amine, lecithin and water.

TABLE 5

| | Lecithin (grams) | | | |
|---|---|---|---|---|
| | 0.00 | 3.02 | 6.04 | 9.33 |
| weight average HLB | 12.5 | 11.5 | 11.1 | 10.7 |
| HLB weight mean square deviation, WMSD | 0.86 | 1.65 | 1.98 | 2.27 |

### Example 10

### Preparation of Micro Emulsions Including Ibuprofen, Fractionated Coconut Oil, Laureth-23 and Lecithin

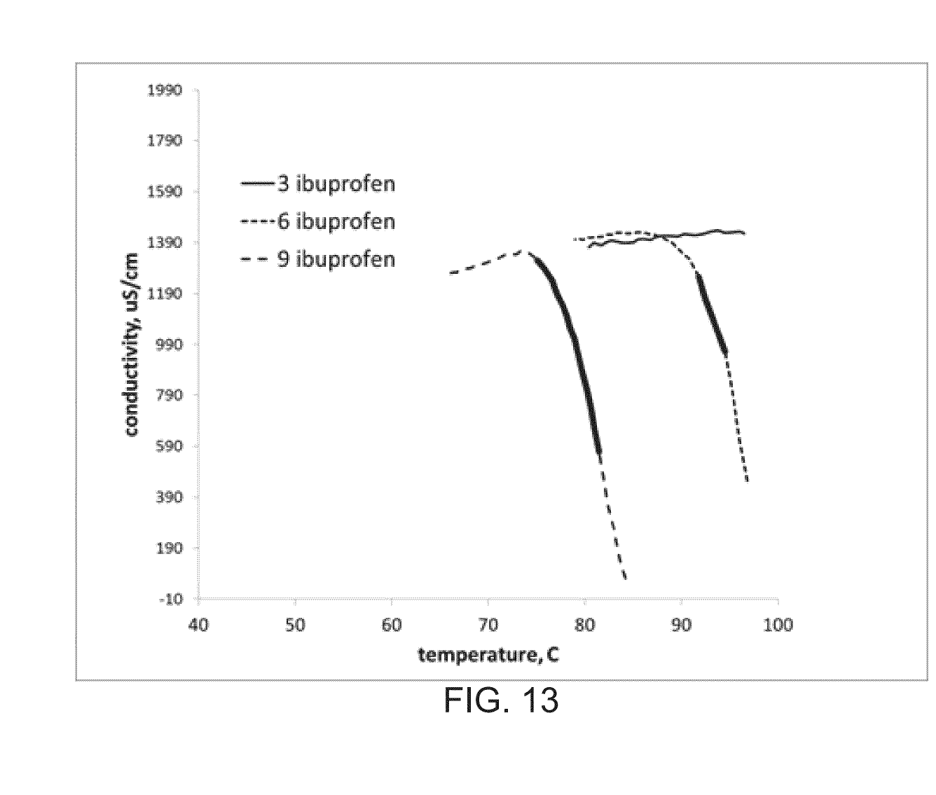

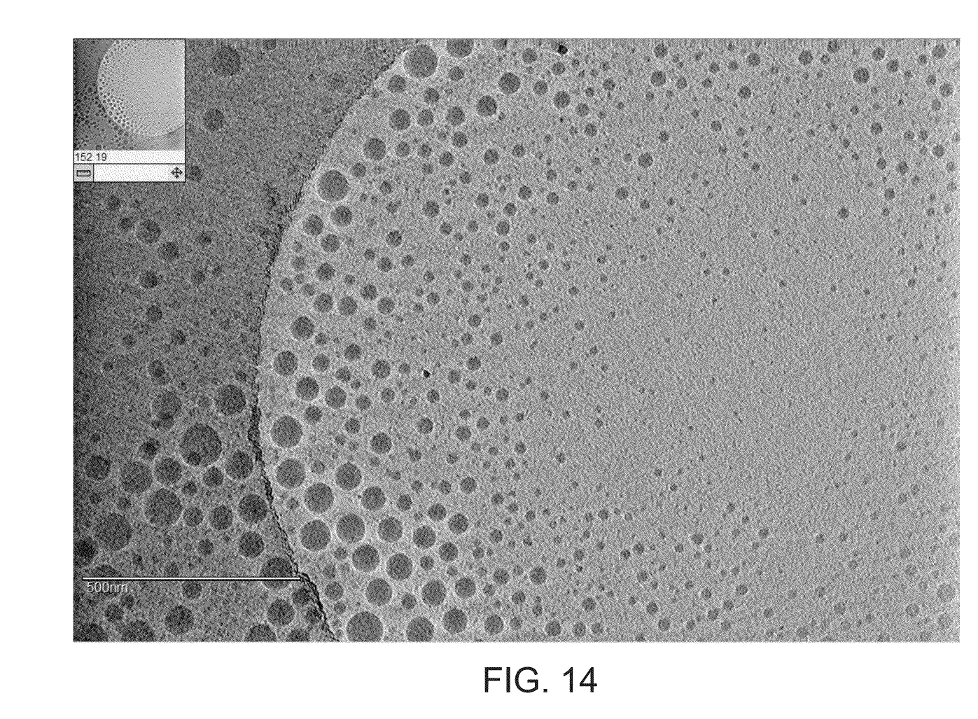

Conductivity (uS/cm) vs. temperature (° C.) was measured for 12.5 grams laureth-23, 12.1 grams Alcolec XTRA-A lecithin, 50.0 grams fractionated coconut oil, 3.0 grams ibuprofen (available from the Walgreen Company, Deerfield, Ill.), and 60.2 grams water. Conductivity remained at about 1400 μS/cm in the temperature range from about 80° C. to about 96° C., and the appearance of the composition remained opaque. Subsequently, water that evaporated was replaced, 3.0 grams of ibuprofen added, and conductivity (uS/cm) vs. temperature (° C.) measured again. In this case, the conductivity increased monotonically as the composition cooled from about 97° C. to about 87° C., and the composition was transparent between about 92° C. and about 95° C. In a subsequent experiment, conductivity (uS/cm) vs. temperature (° C.) was measured for 12.1 grams laureth-23, 12.1 grams Alcolec XTRA-A lecithin, 51.0 grams fractionated coconut oil, 9.1 grams ibuprofen, and 60.1 grams water. Conductivity increased monotonically as the mixture cooled from about 84° C. to about 75° C., and the composition was transparent between about 75° C. and about 82° C. Plots of conductivity (uS/cm) vs. temperature (° C.) are shown in FIG. 13. Weight average HLB and WMSD_{HLB} values for the compositions were calculated using HLB for laureth-23=17.9 (calculated using Molecular Modeling Pro software, version 5.22), HLB for Alcolec XTRA-A lecithin=2 (information from the manufacturer's data sheet), and for HLB for ibuprofen=3.0 (calculated using Molecular Modeling Pro software, version 5.22). Weight average HLB values for compositions with 3.0 grams, 6.0 grams, and 9.0 grams ibuprofen were 9.3, 8.7, and 8.1, respectively, and WMSD_{HLB} values were 6.6, 6.8, and 6.9, respectively. This Example demonstrates that adding ibuprofen to a mixture of fractionated coconut oil, lecithin, and polysorbate-80 decreases the weight average HLB value, decreases the phase inversion temperature, slightly increases the HLB polydispersity, and promotes lamellar character as evidenced by an increase in the microemulsion temperature range.

### Example 11

### Preparation of a Nanoemulsion Including Ibuprofen, Fractionated Coconut Oil, Laureth-23 and Lecithin

A mixture of 12.1 grams laureth-23, 12.05 grams Alcolec XTRA-A lecithin, 9.05 grams ibuprofen (available from the Walgreen Company, Deerfield, Ill.), 51.0 grams fractionated coconut oil, and 60.1 grams of water was heated to about 90° C. and allowed to cool while stirring. The weight average HLB and WMSD_{HLB} values for the composition were 8.1 and 6.9, respectively. When the composition was a transparent amber microemulsion at about 81° C., 78 grams of the composition was poured into 380 grams of stirring water at about 4° C. to give a slightly yellowish, low viscosity, translucently opaque dispersion sample. Cryo-TEM of the sample showed that the dispersion consisted of filled nanoemulsion droplets with diameters less than 100 nm. This Example demonstrates that a microemulsion with relatively less lamellar character decomposes by cooling and dilution to give a dispersion of filled nanoemulsion droplets in a process of Phase Inversion Temperature (PIT) emulsification.

### Example 12

### Preparation of Microemulsions Including Fractionated Coconut Oil, Polysorbate-80, Lecithin and Ibuprofen and a Low Viscosity Oil in Water Emulsion There From

Fractionated coconut oil (about 60 grams of Lotioncrafter FCO), polysorbate 80 (about 12 grams of Lumisorb PSMO-2OK), lecithin (about 12 grams of Alcolec XTRA-A), ibuprofen (about 10 grams, purified Walgreen's brand) and distilled water (about 61 grams) were weighed into a 250 mL beaker. The contents were heated to about 80° C., allowed to cool to the point at which the composition was most transparent (about 66° C.), and the composition was rapidly poured into about 450 grams of cold (about 4° C.) distilled water to give an oil in water emulsion with about 1.7% ibuprofen. When allowed to stand overnight, the product oil in water emulsion separated to give a cream layer. Creaming of the emulsion prepared with about 1.7% ibuprofen is indicative of large particle size dispersoids with particle size greater than about 300 nm, consistent with the formation of large, multilamellar vesicles.

This example demonstrates that emulsions with concentrations of ibuprofen greater than about 1.5% prepared from intermediate microemulsions with strong lamellar character are unstable with respect to separation.

### Example 13

### Preparation of a Low Viscosity Sprayable Composition Including Niosomes of Ibuprofen, Fractionated Coconut Oil, Laureth-23 and Lecithin

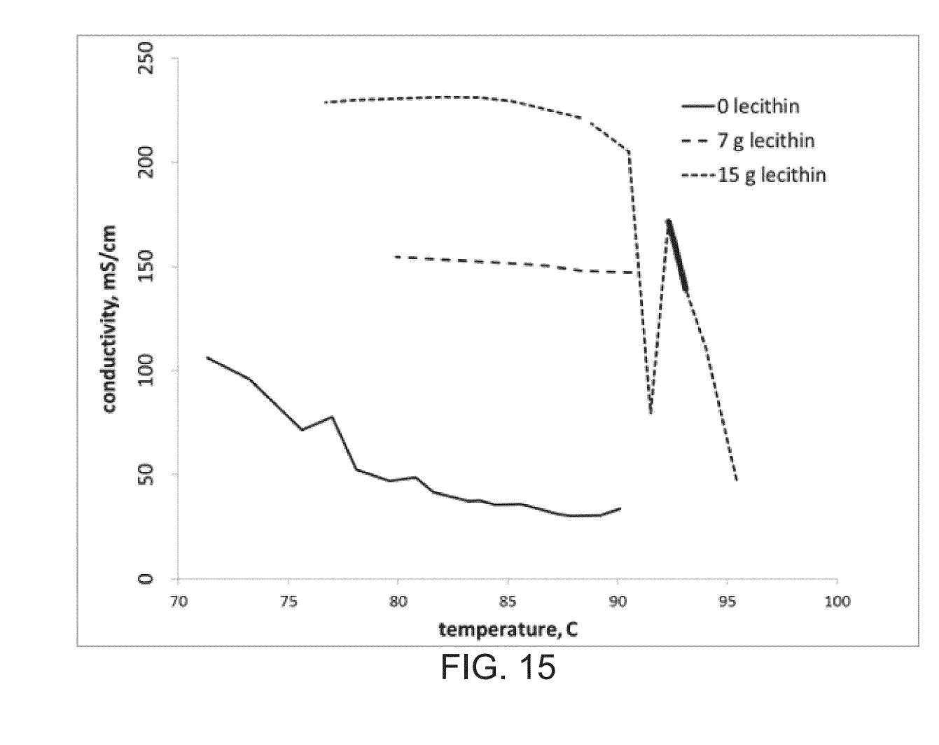

Ibuprofen (9 grams, purified Walgreen's, available from the Walgreen Company, Deerfield, Ill.), fractionated coconut oil (51 grams, Lotioncrafter FCO), laureth-23 (12 grams Lotioncrafter), and 60 grams of water were heated and the appearance and conductivity measured as the sample cooled. After the first heating and cooling cycle, about 7 grams of lecithin (Alcolec XTRA-A) was added, water replaced, and heating and cooling repeated. After the second heating cycle additional lecithin was added, to about 15 grams total. The final composition with 15 grams of lecithin appeared fairly transparent between about 92° C. and about 94° C. Plots of conductivity (uS/cm) vs. temperature (° C.) are shown in FIG. 15. The region where each composition was transparent or hazy transparent is indicated by wide lines in the plots.

The composition with 9 grams ibuprofen, 51 grams fractionated coconut oil, 12 grams laureth-23, 15 grams of lecithin and 60 grams of water was reheated after replacing lost water and when about 94° C., was poured into 167 grams of distilled water to give a low viscosity, sprayable composition. When drops of the emulsion were added to distilled water, a smoky transparent bluish appearing mixture was obtained, indicating small dispersoid particle size. After about 6 weeks, there was no sign of phase separation or deposition of solids.

This Example demonstrates that an oil in water dispersion with a concentration of ibuprofen greater than 2.5% prepared from intermediate micro emulsions with weak lamellar character exhibit fine dispersoid particle size and are stable with respect to separation.

### Example 14

### Preparation of a Topical Lotion Composition Including Niosomes of Ibuprofen, Isopropyl Myristate, Laureth-23, Sorbitan Monostearate, Lecithin and Mineral Oil

Isopropyl myristate (50 grams, Lotioncrafter), laureth-23 (12 grams, Lotioncrafter), sorbitan monostearate (6 grams, Lotioncrafter), ibuprofen (10 grams, purified from Walgreen's brand of 200 mg generic tablets by extracting with 91% isopropanol/water and recrystallized from isopropanol/water, available from the Walgreen Company, Deerfield, Ill.)) and distilled water (60 grams) were weighed into a 250 mL beaker. The contents were heated to about 93° C. and allowed to cool to about 63° C. while stirring with a magnetic stir bar and logging the temperature and conductivity. After cooling and replacing water, about 2 grams of Alcolec XTRA-A lecithin and 3 grams of Crafter's Choice Lavender Fields Fragrance Oil 99 (available from Wholesale Supplies, Broadview Heights, Ohio) was added and the temperature scan repeated from about 95° C. to about 57° C. The composition was transparent between about 65° C. and about 75° C., with some wispy silky swirls growing in as the temperature dropped below about 73° C., becoming isotropic again at about 71° C. After cooling to about 45° C., 15 grams of Crafter's Choice mineral oil Wholesale Supplies Plus, Inc.) was added and the conductivity monitored between about 60° C. and about 94° C. In this case the composition was very transparent between about 69° C. and about 78° C., with silky swirling anisotropic haze between about 73° C. and about 74° C.

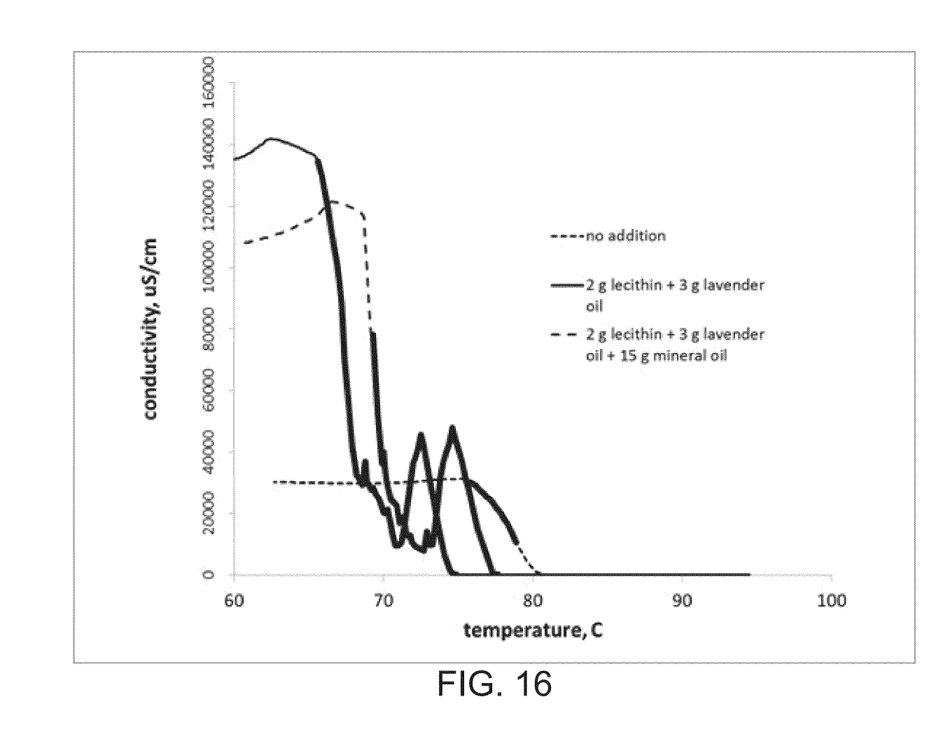

Plots of conductivity versus temperature for compositions with isopropyl myristate, laureth-23, sorbitan stearate, lecithin, mineral oil and ibuprofen are shown in FIG. 16. The regions where each composition was transparent or hazy transparent is indicated by wide lines in the plots. After recording the conductivity and temperature, water was replaced and the composition was reheated and quenched from about 75.5° C. into a frozen jacketed plastic beer mug and stirred with a freeze pop to give a translucent beige amber yield stress fluid (that is, a Bingham plastic) which liquefies upon rubbing. The product yield stress fluid was stored at room temperature and after 6 weeks, showed no signs of separation. When a drop of the yield stress fluid was added to distilled water, a smoky transparent bluish appearing mixture was obtained, indicating small dispersoid particle size. After 6 weeks, there was no sign of phase separation or deposition of solids.

This Example demonstrates that an oil in water yield stress fluid with a concentration of ibuprofen greater than 7% prepared from intermediate micro emulsions with weak lamellar character exhibits fine dispersoid particle size and is stable with respect to separation.

### Example 15

### Microemulsions with Cholesterol, Polysorbate-80, Ceteareth-30, Lecithin, Fractionated Coconut Oil, and Ibuprofen

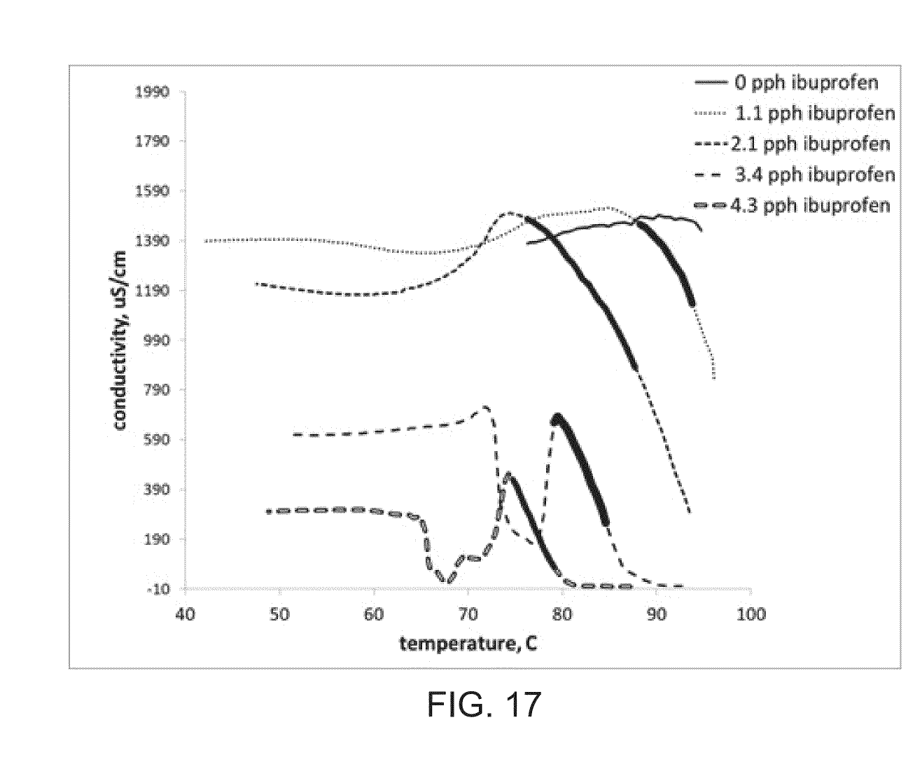

The conductivity of a mixture of 12.1 grams polysorbate-80, 12.0 grams Alcolec XTRA-A lecithin, 50.0 grams fractionated coconut oil, 1.6 grams ceteareth-30, 3.0 grams cholesterol and 61.0 grams water was measured as it cooled from about 95° C. to about 76° C. The weight average HLB and the HLB polydispersity as WMSD_{HLB} were calculated using HLB values for polysorbate-80, lecithin, ceteareth-30, and ibuprofen equal 16.5, 2.0, 17.6, and 3.0, respectively (all HLB values were calculated using Molecular Modeling Pro software, version 5.22 except for Alcolec XTRA-A lecithin, which is based on information from the manufacturer's data sheet). The weight average HLB was 9.8 and WMSD_{HLB} was 5.5. Throughout the temperature range, the composition remained opaque and the conductivity remained near 1400 μS/cm. After replacing water that had evaporated, 1.5 grams of ibuprofen was added (ibuprofen concentration=1.1 pph (parts per hundred) overall and 1.9 weight percent of non-volatile content) and the sample was reheated and allowed to cool from about 96° C. to about 42° C. The conductivity increased monotonically to 1515 μS/cm at about 86° C., and slowly declined as it cooled to lower temperatures. The composition was hazy but transparent in the temperature range between about 88° C. and about 94° C., After replacing water that had evaporated, an additional 1.5 grams of ibuprofen was added (2.1 pph ibuprofen overall and 3.7 weight percent of non-volatile content) and the conductivity scan repeated. The conductivity reached a maximum of about 1500 μS/cm at about 74° C. and showed a proportionately greater decline upon cooling further, consistent with the further development and maintenance of greater lamellar character. The composition was hazy and transparent in the temperature range from about 76° C. to about 88° C. Evaporated water was replaced, the mixture was reheated, and when at about 89° C., a 14.7 grams portion was poured from about 89° C. into 90 grams cold (about 7° C.) stirring water to give an opaque beige dispersion. The specific turbidity of the dispersion sample determined as the slope of turbidity vs. concentration of non-water ingredients in the concentration range between about 0 and about 600 ppm (parts per million) was found to be 0.36 nephelometric turbidity units (NTU) per ppm non-water. To the remaining microemulsion composition, an additional 1.6 grams of ibuprofen was added (3.4 pph overall and 5.8 weight percent of non-volatile content) and the conductivity (uS/cm) vs. temperature (° C.) measured again. The conductivity never exceeded 750 μS/cm and plateaued at about 610 μS/cm below about 60° C., indicating still greater lamellar character. The composition was transparent between about 80° C. and about 85° C. After replacing evaporated water, the sample was reheated and when at about 82° C., a 12.0 grams portion was poured into cold into 90 grams cold (about 7° C.) stirring water to give an opaque beige dispersion. The specific turbidity of the new dispersion sample was 0.38 NTU/ppm non-water, indicating little increase in dispersion particle size compared to the first dispersion sample. To the remaining microemulsion composition, an additional 1.2 grams of ibuprofen was added (4.3 pph overall and 7.4 weight percent of non-volatile content) and the conductivity (uS/cm) vs. temperature (° C.) measured again. The conductivity never exceeded 450 μS/cm consistent with still greater lamellar character, and the composition was transparent between about 75° C. and about 79° C. After replacing evaporated water, the sample was reheated and when at about 82° C., a 22.5 grams portion was poured into cold into 180 grams cold (about 7° C.) stirring water to give an opaque beige dispersion. The specific turbidity of the new dispersion sample was 0.62 NTU/ppm non-water, indicating larger particle size, consistent with the formation of larger, multi-lamellar liposomes. Values of weight average HLB and WMSD_{HLB} for the five compositions with 0, 1.1 pph ibuprofen, 2.1 pph ibuprofen, 3.4 pph ibuprofen, and 4.3 pph ibuprofen are shown in Table 6 and plots of conductivity (uS/cm) vs. temperature (° C.) are shown in FIG. 17. This Example demonstrates that the addition of ibuprofen to a composition of water, polysorbate-80, lecithin, ceteareth-20, and cholesterol has the effect of increasing the lamellar character and promoting the generation of a microemulsion.

TABLE 6

| | pph of ibuprofen | | | | |
|---|---|---|---|---|---|
| | 0.00 | 1.1 | 2.1 | 3.4 | 4.3 |
| weight average HLB | 9.8 | 9.4 | 9.1 | 8.7 | 8.5 |
| HLB weight mean square deviation, WMSD | 5.5 | 5.6 | 5.7 | 5.9 | 5.9 |

### Example 16

### Preparation of Liposomes Including Cholesterol, Polysorbate-80, Ceteareth-30, Lecithin, Fractionated Coconut Oil, and Ibuprofen

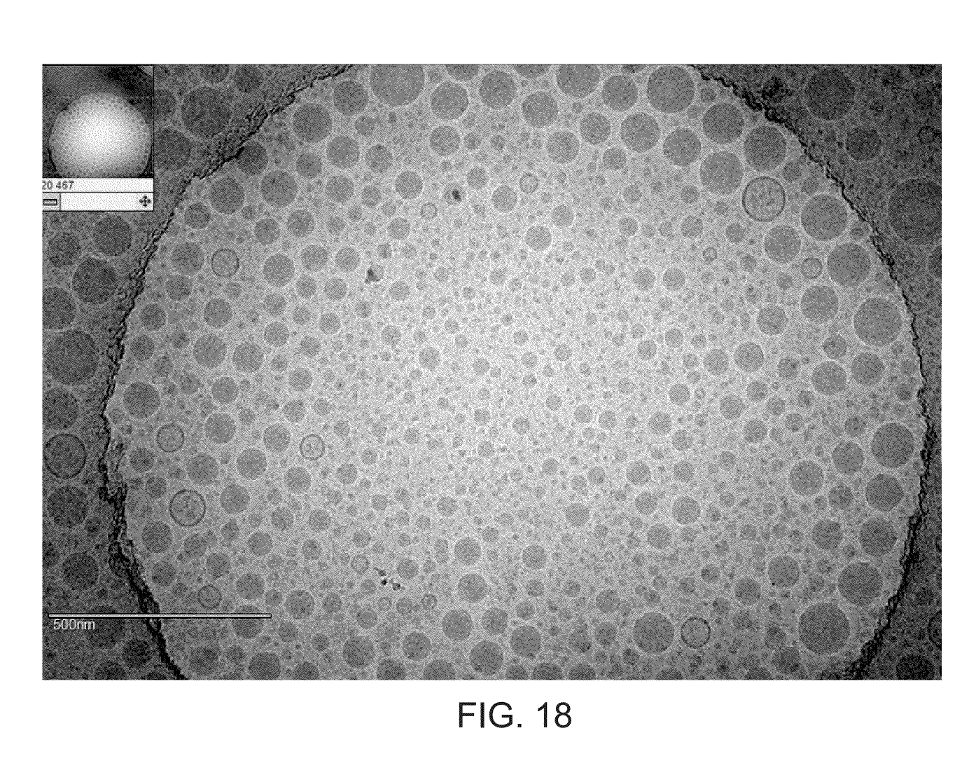

The conductivity of a mixture of 12.0 grams polysorbate-80, 12.0 grams Alcolec XTRA-A lecithin, 50.0 grams fractionated coconut oil, 18 grams ceteareth-30, 3.0 grams cholesterol, 4.5 grams ibuprofen (available from the Walgreen Company, Deerfield, Ill.), and 61.0 grams water was measured as it cooled from about 90° C. to about 56° C. This composition has 3.1 pph ibuprofen overall, 5.4% of ibuprofen in the non-volatile content, weight average HLB equal to 8.8, oil to surfactant ratio (including ibuprofen) equal to 1.66:1, and HLB polydispersity as WMSD_{HLB} equal to 5.9. The maximum conductivity was about 920 μS/cm, the conductivity plateaued at between 700 and 750 μS/cm below about 60° C., and the composition was transparent and hazy between about 78° C. and about 84° C., indicating a microemulsion. After measuring conductivity, water that evaporated was replaced, the sample was re-heated, and when at about 78° C., 44.3 grams of microemulsion was poured into 160 grams of stirring cold (about 7° C.) water to give an opaque beige dispersion. The specific turbidity of the dispersion was determined to be 0.40 NTU/ppm non-water and the volume average particle size was determined by DLS to be 109.7 nm with polydispersity=0.236. When examined by cryo-TEM, the dispersed particles were found to contain both liposomes and filled nanoemulsion droplets with particle sizes less than about 150 nm as shown in FIG. 18.

### Example 17

### Microemulsions with Cholesterol, Polysorbate-80, Ceteareth-30, Lecithin, Fractionated Coconut Oil, Naproxen, and Octanoic Acid

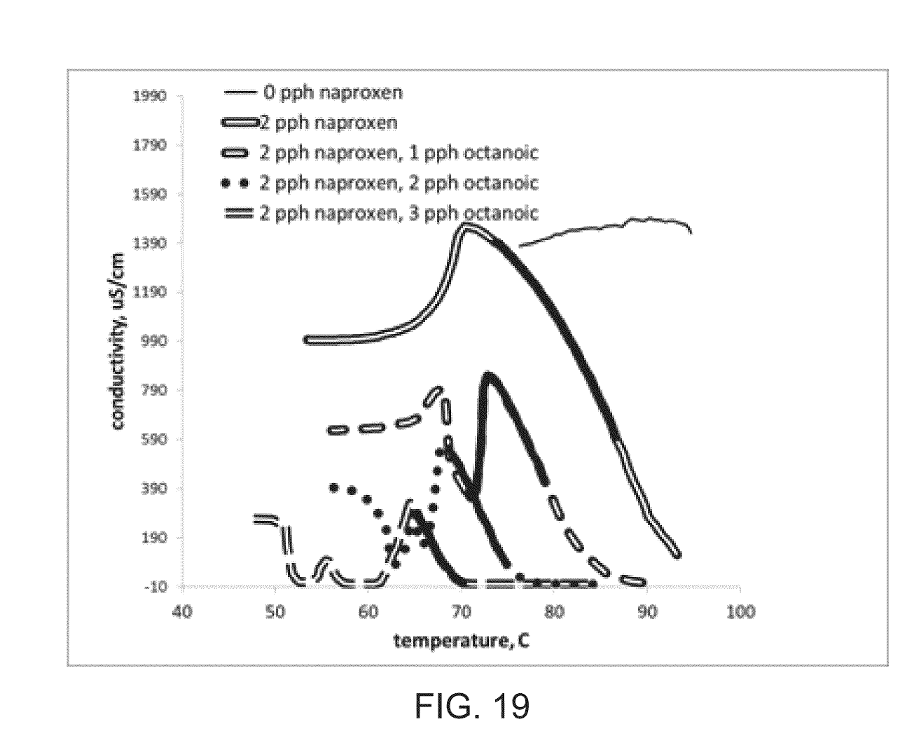

The conductivity of a mixture of 12.1 grams polysorbate-80, 12.0 grams Alcolec XTRA-A lecithin, 50.0 grams fractionated coconut oil, 1.6 grams ceteareth-30, 3.0 grams cholesterol and 61.0 grams water was measured as it cooled from about 95° C. to about 76° C. The weight average HLB and the HLB polydispersity as WMSD_{HLB} were calculated using HLB values for polysorbate-80, lecithin, ceteareth-30, naproxen, and octanoic acid equal to 16.5, 2.0, 17.6, 3.5. and 4.5. respectively (all HLB values were calculated using Molecular Modeling Pro software, version 5.22 except for Alcolec XTRA-A lecithin, which is based on information from the manufacturer's data sheet). The weight average HLB was 9.8 and WMSD_{HLB} was 5.5. Throughout the temperature range, the composition remained opaque and the conductivity remained near 1400 μS/cm. After replacing water that had evaporated, 3.0 grams of naproxen (available from the Walgreen Company, Deerfield, Ill.), was added (naproxen concentration=2.1 pph) and the sample was reheated and allowed to cool, giving a microemulsion in the temperature range between about 74° C. and about 87° C. After replacing water that had evaporated, 1.55 grams of octanoic acid was added (2.1 pph naproxen and 1.1 pph octanoic acid) and the conductivity scan repeated showing a microemulsion between about 69° C. and about 79° C. and maximum conductivity=850 μS/cm. Evaporated water was replaced, the mixture was reheated, and when at about 89° C., a 18.4 grams portion was poured from about 80° C. into 89 grams cold (about 7° C.) stirring water to give an opaque beige dispersion. The specific turbidity of the dispersion sample determined as the slope of turbidity vs. concentration of non-water ingredients in the concentration range between about 0 and about 600 ppm was found to be 0.41 nephelometric turbidity units (NTU) per ppm non-water. The volume average particle size of the dispersion was measured using a Beckman Coulter Delsa Nano C dynamic light scattering (DLS) instrument and found to be 93.2 nm with a polydispersity of 0.253. To the remaining microemulsion composition, additional octanoic acid (1.3 grams) was added to give octanoic acid concentration=2.1 pph and the conductivity (uS/cm) vs. temperature (° C.) measured again. The conductivity never exceeded 600 μS/cm, indicating still greater lamellar character, and the composition was transparent between about 69° C. and about 74° C., indicating a microemulsion. After replacing evaporated water, the sample was reheated and when at about 75° C., a 21.0 grams portion was poured into cold into 90 grams cold (about 7° C.) stirring water to give an opaque beige dispersion with specific turbidity=0.78 NTU/ppm. The volume average particle size and polydispersity of the dispersion were found to be 140.2 nm and 0.194 by DLS. To the remaining microemulsion composition, an additional 1.12 grams of octanoic acid was added (3.1 pph octanoic acid) and the conductivity (uS/cm) vs. temperature (° C.) measured again. The conductivity never exceeded 330 μS/cm, consistent with still greater lamellar character, and the composition was transparent between about 65° C. and about 70° C. After replacing evaporated water, the sample was reheated and when at about 70° C., a 19.6 grams portion was poured into cold into 90 grams cold (about 7° C.) stirring water to give an opaque beige dispersion. The specific turbidity of the new dispersion sample was 0.1.10 NTU/ppm non-water, and the volume average particle size was 166.4 nm with polydispersity=0.223. Values of weight average HLB and WMSD_{HLB} for the four compositions with 0, 1.1 pph 2.1 pph, and 3.1 pph are shown in Table 7 along with values of the specific turbidity and particle size of dispersions made from the microemulsions. Plots of conductivity (uS/cm) vs. temperature (° C.) are shown in FIG. 19. This Example demonstrates that the addition of octanoic acid to a composition of water, polysorbate-80, lecithin, ceteareth-20, cholesterol and naproxen has the effect of increasing the lamellar character and promoting the generation of a microemulsion.

TABLE 7

| | pph of octanoic acid | | | |
|---|---|---|---|---|
| | 0.00 | 1.1 | 2.1 | 3.1 |
| weight average HLB | 9.1 | 8.8 | 8.6 | 8.4 |
| HLB weight mean square deviation, | 5.7 | 5.6 | 5.6 | 5.6 |
| WMSD | | | | |
| Specific turbidity, NTU/ppm | | 0.41 | 0.78 | 1.10 |
| Particle size, nm | | 93.2 | 140.2 | 166.4 |

### Example 18

### Preparation of Liposomes Including Cholesterol, Polysorbate-80, Ceteareth-30, Lecithin, Fractionated Coconut Oil, Naproxen, and Octanoic Acid

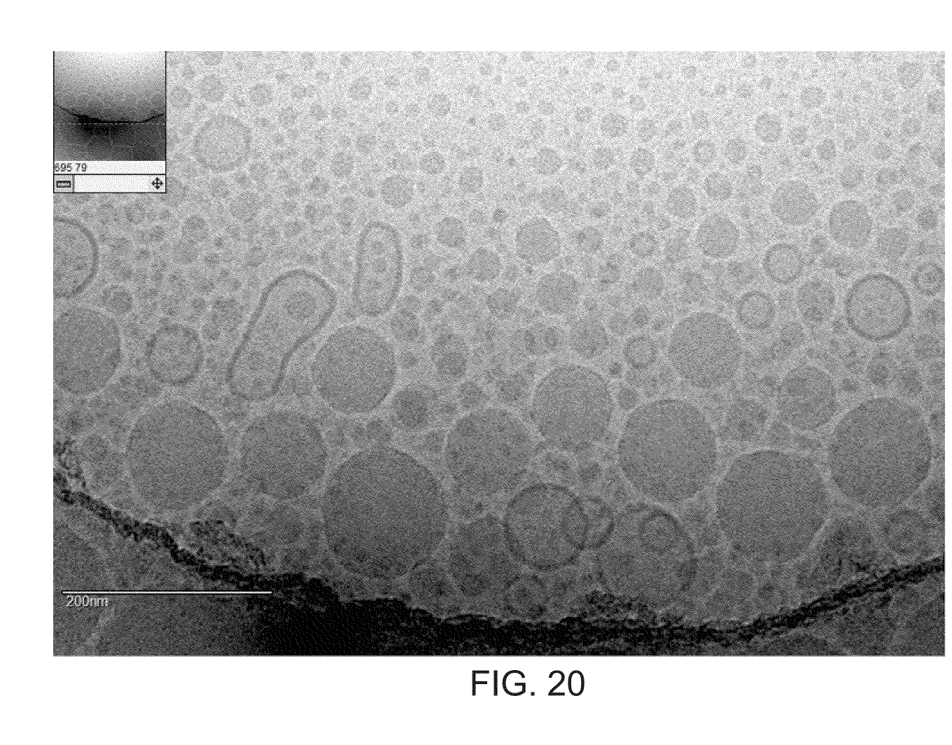

The conductivity of a mixture of 12.1 grams polysorbate-80, 12.2 grams Alcolec XTRA-A lecithin, 50.0 grams fractionated coconut oil, 1.5 grams ceteareth-30, 3.0 grams cholesterol, 3.0 grams naproxen, 4.5 grams octanoic acid and 60.4 grams water was measured as it cooled from about 72° C. to about 41° C. This composition has 2.0 pph naproxen (available from the Walgreen Company, Deerfield, Ill.), 3.1 pph octanoic acid, weight average HLB equal to about 8.4, oil to surfactant ratio (including naproxen and octanoic acid) equal to about 1.50:1, and HLB polydispersity as WMSD_{HLB} equal to 5.6. The maximum conductivity was about 375 μS/cm and the conductivity plateaued at about 275 μS/cm below about 60° C. After measuring conductivity, water that evaporated was replaced, the sample was re-heated, and when at about 69° C., 34.1 grams of microemulsion was poured into 160 grams of stirring cold (about 7° C.) water to give an opaque beige dispersion. The specific turbidity of the dispersion was determined to be 1.11 NTU/ppm non-water and the volume average particle size determined by DLS was 165.9 nm with polydispersity=0.201. When examined by cryo-TEM, the dispersed particles were found to contain both liposomes and filled nanoemulsion droplets with particle sizes less than about 200 nm as shown in FIG. 20.

### Example 19

### Application of a Topical Niosomal Composition to Patient in Need of Medication

Approximately 1.5 grams of a niosomal yield stress fluid including ibuprofen prepared as described in Example 4 was applied by rubbing to the swollen popliteal fossa area posterior of the knee of a 56 year old male that had been surgically repaired (38 years postoperative medial menisectomy plus anterior crusciate ligament auto graft, 15 years postoperative anterior crusciate ligament repair by cadaver graft). The patient was complaining of pain in the popliteal fossa region after having ridden a stationary bicycle for 30 minutes at moderate intensity. Approximately 30 minutes after application of ibuprofen containing yield stress fluid, swelling in the popliteal fossa was noticeably reduced and the patient reported that the pain was substantially reduced.

This Example demonstrates that a niosoma oil in water yield stress composition fluid with a concentration of ibuprofen greater than about 7% prepared from intermediate micro emulsions with weak lamellar character exhibits good transcutaneous bioavailability when applied as a topical lotion.

### Example 20

### Application of a Topical Niosomal Composition to a Patient in Need of Medication

Approximately 1.5 grams of a niosomal yield stress fluid including ibuprofen prepared as described in Example 4 was applied by rubbing to the hands of a 60 year old female patient that had pronounced swelling, reduced range of motion, and mild pain in the interphalangeal joint between the fourth middle and fourth proximal phalanxes of one hand. During application, the product became more fluid and felt viscous, then after about 2 minutes the product felt less viscous and more “slippery,” and after about 10 minutes of rubbing the product had essentially disappeared, leaving the skin moist but not sticky. The patient reported reduced swelling, greater range of motion and no pain for the period starting about 30 minutes after application and continuing for about eight hours.

This Example demonstrates that a niosomal oil in water yield stress composition fluid with a concentration of ibuprofen greater than about 7% prepared from intermediate micro emulsions with weak lamellar character exhibits good transcutaneous bioavailability when applied as a topical lotion.

### Example 21

### Preparation of Microemulsions Including Fractionated Coconut Oil, Polysorbate-80, Lecithin, Ibuprofen and Glycerin

Fractionated coconut oil (48.1 grams, product of Crafters Choice, Independence, Ohio), polysorbate 80 (12.1 grams, product of Lotioncrafter, Eastsound, Wash.), lecithin (12.0 grams of Alcolec XTRA-A), ibuprofen (4.64 grams) and distilled water (48.0 grams) were weighed into a 250 mL beaker. The contents were heated to about 87° C. and allowed to cool while stirring with a magnetic stir bar and logging the temperature and conductivity. The composition was a transparent microemulsion between about 69° C. and about 76° C.

In a separate experiment, the conductivity of a mixture of 12.0 grams polysorbate-80, 12.1 grams Alcolec XTRA-A lecithin, 48.1 grams fractionated coconut oil, 4.50 grams ibuprofen, and 40.3 grams water was measured as it cooled from 80° C. to about 38° C. The composition was a transparent microemulsion between about 66° C. and 74° C., and exhibited birefringence between about 66° C. and 70° C., indicating the presence of lamellae.

After measuring conductivity, water that had evaporated was replaced and 20.0 grams of glycerin added, and conductivity measured again as the composition cooled from 81° C. to about 37° C. This composition was a transparent microemulsion between about 52° C. and about 66° C., and exhibited birefringence between about 52° C. and about 64° C., indicating the presence of lamellae.

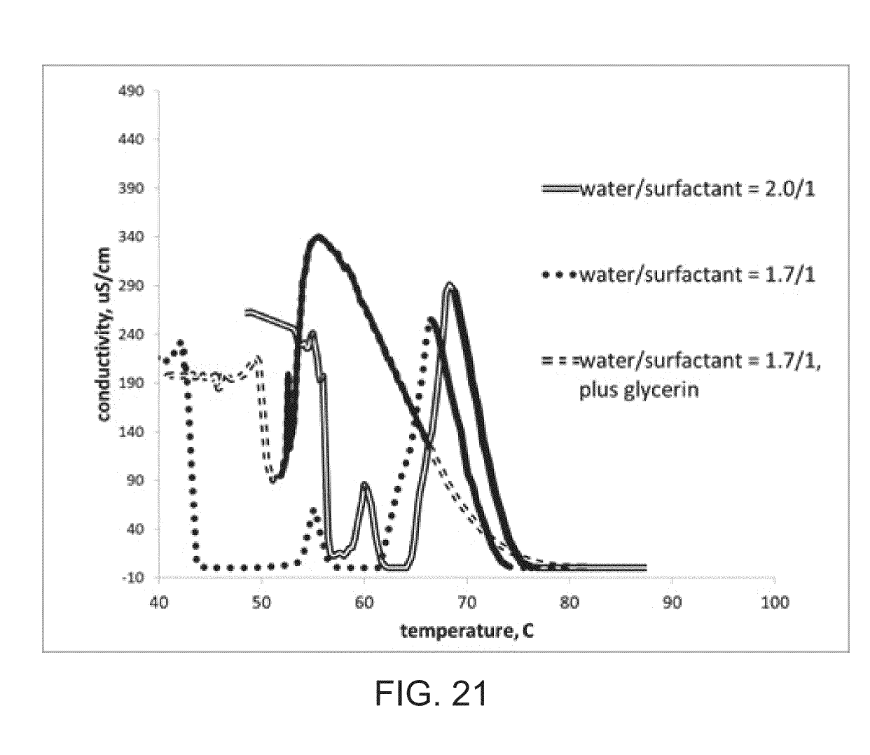

Plots of conductivity (uS/cm) vs. temperature (° C.) for the two compositions are shown in FIG. 21. The regions where each composition was transparent or hazy transparent is indicated by wide lines in the plots. For all three experiments, the ratio of oil to surfactant (including ibuprofen was 1.68:1, the weight average HLB was 8.3, and WMSD_{HLB} was 6.0.

This Example demonstrates that the microemulsion temperature can be reduced by adding glycerin, which is a kosmotrope, to a mixture of oil, surfactant, and water. The lowest temperature at which a composition of fractionated coconut oil, ibuprofen, lecithin and polysorbate-80 is a microemulsion is reduced by about 14° C. when the aqueous phase is changed from water to about a 2/1 mixture of water/glycerin.

### Example 22

### Preparation of Microemulsions of Various Oils with Polysorbate-80, Lecithin and Water with 1:1 Weight Ratio of Oil to Surfactant

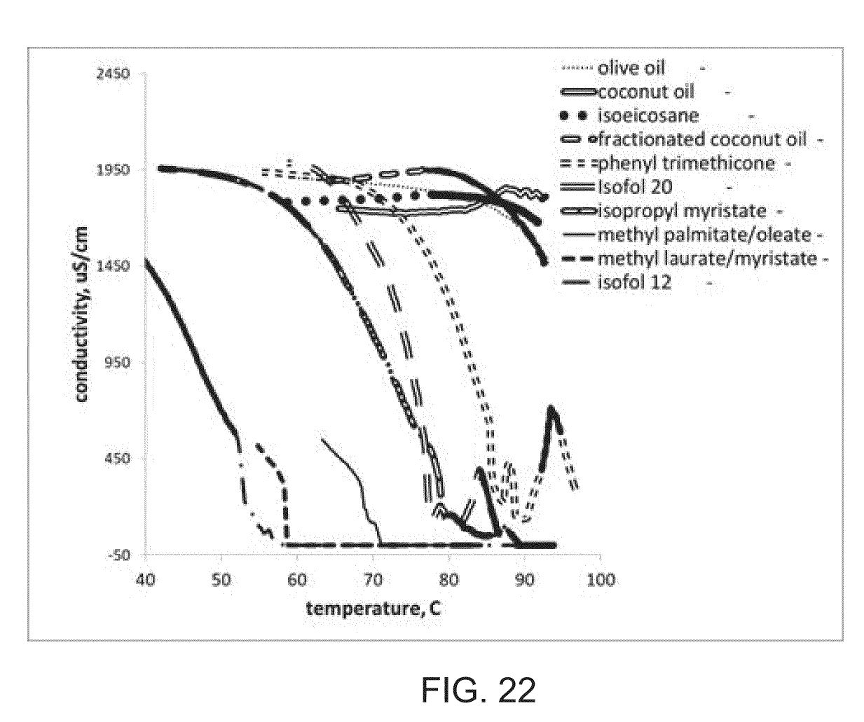

A mixture of 18.0 grams polysorbate-80 (Lotioncrafter, Eastsound, Wash.), 13.5 grams of Alcolec XTRA-A lecithin (American Lecithin, Oxford, Conn.), 90.0 grams of water and 31.5 grams of isopropyl myristate (Lotioncrafter), was heated to about 94° C. and allowed to cool to about 40° C. while stirring with a magnetic stir bar and logging the temperature and conductivity. The cooling composition was observed visually and temperatures at which the composition was a microemulsion were noted. The experiment was repeated with olive oil (Bertolli Extra Virgin olive oil, product of R & B Foods), coconut oil (Nature's Bounty coconut oil, product of Nature's Bounty, Bohemia, N.Y.), isoeicosane, fractionated coconut oil (product of Lotioncrafter, Eastsound, Wash.), phenyl trimethicone (phenyltris(trimethylsiloxy)silane, product of Lotioncrafter, Eastsound, Wash.), Isofol 12 (2-butyl octanol, product of Sasol North America, Houston, Tex.), Isofol 20 (2-octyl decanol, product of Sasol North America), methyl palmitate/oleate (P&G Chemicals methyl ester product CE-1618, available from P&G Chemicals, Cincinnati, Ohio), and methyl laurate/myristate (P&G Chemicals methyl ester product CE-1270). Plots of conductivity (uS/cm) vs. temperature (° C.) for the compositions are shown in FIG. 22. The region where each composition was a transparent or hazy transparent microemulsion is indicated by filled solid lines in the plots. The molecular weight and polarity (as the polarity component of the van Krevelen/Hoftyzer solubility parameter calculated by the method on p. 200-225 of ‘Properties of Polymers’ by D. W. van Krevelen (Elsevier, 1990) using Molecular Modeling Pro software, version 5.22, commercialized by Norgwyn Montgomery Software Inc. ©2003) of oils is shown in Table 8. This Example demonstrates that the relative lamellar character of microemulsions for mixtures of oils with polysorbate-80, lecithin and water increases in the order of olive oil<coconut oil<isoeicosane<fractionated coconut oil<phenyltris(trimethylsiloxy)silane (phenyl trimethicone)<2-octyldodecanol (Isofol 20)<isopropyl myristate<methyl palmitate/oleate<methyl laurate/myristate<2-butyloctanol (Isofol 12).

TABLE 8

| | molecular | |
|---|---|---|
| oil | weight | polarity |
| 2-butyl octanol (isofol 12) | 186 | 2.26 |
| benzyl benzoate | 212 | 2.79 |
| methyl laurate | 214 | 1.97 |
| isopropyl myristate | 270 | 1.56 |
| isoeicosane (calculated as 2 methyl nonadecane) | 283 | 0.00 |
| methyl oleate | 296 | 1.44 |
| 2-octyl-1-dodecanol (isofol 20) | 299 | 1.41 |
| tris(trimethylsiloxy)phenyl silane | 373 | 1.81 |
| fractionated coconut oil (calculated as average of | 513 | 1.62 |
| tricaprylin and tricaprin) | | |
| coconut oil (calculated as trilaurin) | 639 | 1.25 |
| olive oil (calculated as triolein) | 885 | 0.89 |

### Example 23

### Preparation of Microemulsions Including Fractionated Coconut Oil, Polysorbate-80, and Lecithin

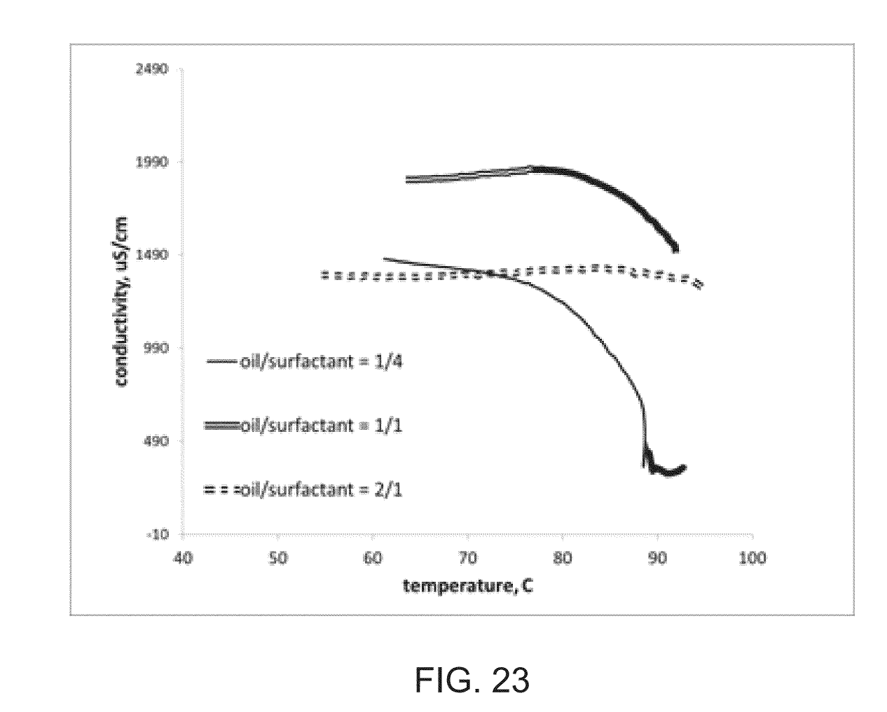

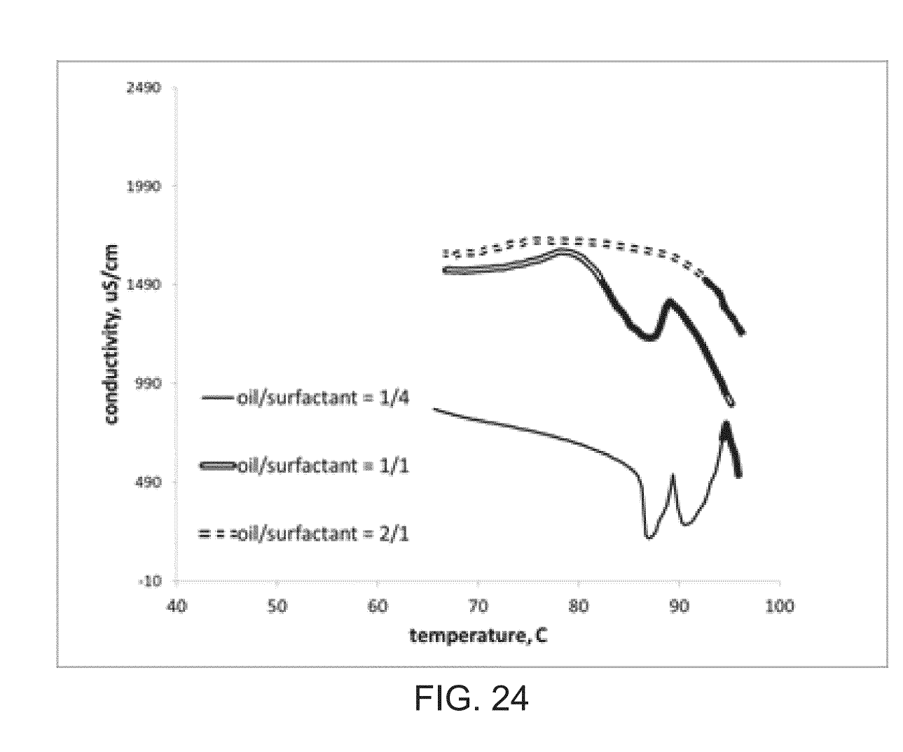

A mixture of fractionated coconut oil (7.9 grams, product of Lotioncrafter), polysorbate-80 (18.4 grams, product of Lotioncrafter), lecithin (13.5 grams Alcolec XTRA-A, product of American Lecithin, Oxford, Conn.) and water (89.9 grams) were heated to 94° C. and allowed to cool while measuring temperature and conductivity. The ratio of low HLB surfactant to high HLB surfactant was about 0.75:1 and the ratio of oil to surfactant was about 0.25:1. The mixture was a microemulsion above about 89° C. as evidenced by an isotropic and moderately transparent appearance. Subsequently, fractionated coconut oil was added so that the ratio of oil to surfactant was about 1:1 and then additional fractionated coconut oil was added so that the ratio of oil to surfactant was about 2:1. For each cooling composition, electrical conductivity was measured and the temperatures at which a microemulsion existed were noted. Plots of conductivity (uS/cm) vs. temperature (° C.) are shown in FIG. 23. The region where each composition was a transparent or hazy transparent microemulsion is indicated by filled solid lines in the plots. In a second series of experiments, conductivity was measured for a composition of 9.0 of fractionated coconut oil, 18.0 grams polysorbate-80, 18.2 grams lecithin, and 90.0 grams of water as it cooled from about 96° C. to about 66° C. The ratio of low HLB surfactant to high HLB surfactant was about 1:1 and the ratio of oil to surfactant was about 0.25:1. The mixture was a microemulsion above about 94° C. as evidenced by an isotropic and moderately transparent appearance. Subsequently, fractionated coconut oil was added so that the ratio of oil to surfactant was about 1:1 and then additional fractionated coconut oil was added so that the ratio of oil to surfactant was about 2:1. For each cooling composition, electrical conductivity was measured and the temperatures at which a microemulsion existed were noted. Plots of conductivity (uS/cm) vs. temperature (° C.) are shown in FIG. 24. The region where each composition was a transparent or hazy transparent microemulsion is indicated by filled solid lines in the plots. The conductivity for compositions with about a 1:1 weight ratio of lecithin to polysorbate-80 are relatively lower than those with about a 0.75:1 weight ratio of lecithin to polysorbate-80 at oil to surfactant ratios of about 1:4 and about 1:1, indicating greater lamellar character. Greater lamellar character of the composition with about 1:1 weight ratio of lecithin to polysorbate-80 and oil to surfactant ratios of about 2:1 compared to the composition with about 0.75:1 weight ratio of lecithin to polysorbate-80 and oil to surfactant ratios=2:1 is evidenced by the formation of a microemulsion (conductivity overall was slightly higher because of the higher concentration of ionic species from lecithin). This Example demonstrates that the susceptibility of a mixture of surfactants towards forming lamellar microemulsion phases increases as the weight ratio of low HLB surfactant to high HLB surfactant increases from about 0.75:1 to about 1:1.

### Example 24

### Determination of Apparent pKa for a Liposomal Ibuprofen Dispersion

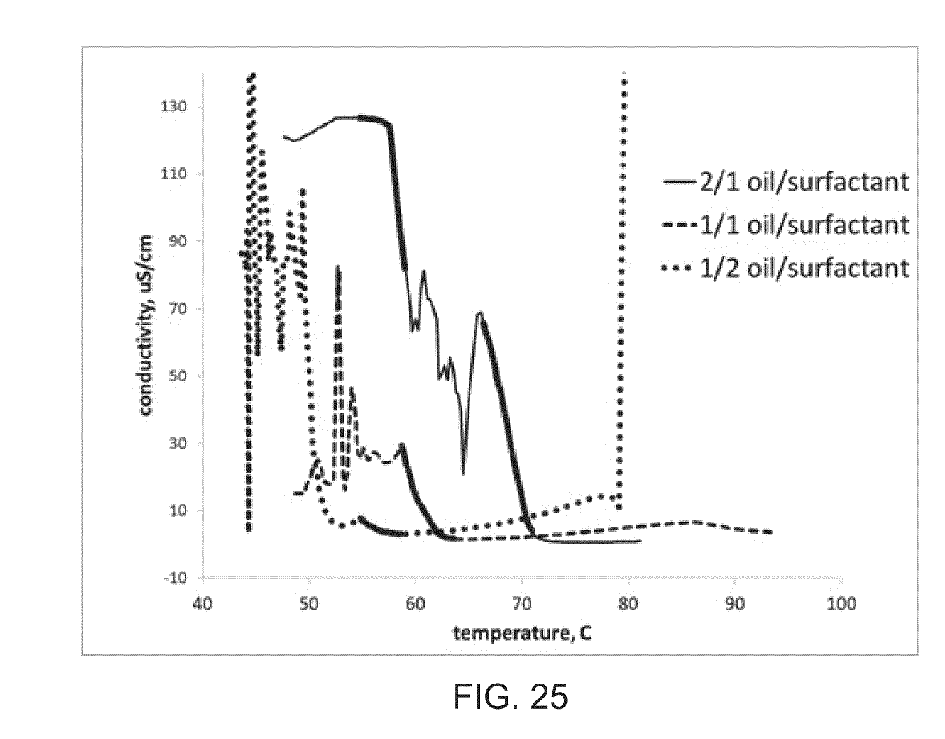

A mixture of phenyl trimethicone (30.0 grams, product of Lotioncrafter, Eastsound, Wash.), cyclopentasiloxane (30.1 grams, product of Lotioncrafter, Eastsound, Wash.), laureth-23 (18.1 grams, product of Lotioncrafter, Eastsound, Wash.), laureth-3 (12.0 grams, product of Making Cosmetics, Snoqualmie Wash.), ibuprofen (10.6 grams) and water (66.0 grams) were heated to about 81° C. and allowed to cool while measuring temperature and conductivity. The ratio of oil to surfactant (including ibuprofen) was about 1.50:1 and weight percent water in the composition was about 37%. The mixture was a microemulsion between about 55° C. to about 59° C. and about 66° C. to about 71° C. as evidenced by an isotropic and moderately transparent appearance. The presence of a negative peak in the plot of conductivity (uS/cm) vs. temperature (° C.) from about 58° C. to about 66° C. is consistent with the presence of a lamellar phase microemulsion. A plot of conductivity (uS/cm) vs. temperature (° C.) is shown in FIG. 25. The presence of a negative peak in the plot of conductivity (uS/cm) vs. temperature (° C.) from about 58° C. to about 66° C. is consistent with the presence of a lamellar phase microemulsion. In a separate experiment, conductivity (uS/cm) vs. temperature (° C.) was measured for a similar composition with ratio of oil to surfactant (not including ibuprofen)=0.74:1 and 48 weight percent water (15.1 grams phenyl trimethicone, 15.1 grams cyclopentasiloxane, 18.0 grams laureth-23, 12.0 grams laureth-3, 10.6 grams ibuprofen and 66.0 grams water). The conductivity of the second sample was depressed relative to the first, indicating greater lamellar character. To 63.5 grams of this composition were added 5.6 grams additional laureth-3, 30.9 grams additional water, 4.9 grams additional ibuprofen, and 8.4 grams of laureth-30 (product of Making Cosmetics, Snoqualmie Wash.) to give a composition with 8.4 grams laureth-30, 8.4 grams laureth-23, 11.1 grams laureth-3, 7.0 grams phenyl trimethicone, 7.0 grams cyclopentasiloxane, 9.8 grams ibuprofen, and 61.6 grams water. The ratio of oil to surfactant (not including ibuprofen) was about 0.37:1 and weight percent water in the composition was about 54%. Conductivity of the sample was measured as it cooled from about 96° C. and is shown in FIG. 25. The sample was a microemulsion in the temperature range from about 55° C. to about 59° C. In spite of the greater concentration of water and lower concentration of oil, conductivity of the sample was depressed relative to the sample without additional laureth-3, laureth-30, ibuprofen and water, indicating still greater lamellar character. After measuring conductivity, water that evaporated was replaced, the sample was reheated, and when at about 57° C. was poured into 403 grams of cold water to give an opaque white dispersion containing 1.9 weight percent ibuprofen. After standing overnight, the dispersion was decanted from a small amount of ibuprofen crystals that had formed and the sample was potentiometrically titrated with NaOH. The weight percent ibuprofen in the sample by titration was about 1.8% (94% retention) and the apparent pKa value (determined as the pH at the half titration point) was about 6.2. The concentration of oil (phenyl trimethicone plus cyclopentasiloxane) in the liposomal dispersion was about 3.1 weight percent. After standing for about 7 days further, additional crystals that formed were removed by decanting and the dispersion was titrated again to determine the concentration of ibuprofen and apparent pKa value. The concentration of ibuprofen was about 1.0 weight percent (about 55% retention) and the apparent pKa value was about 6.4.

### Example 25

### Preparation of Magnetoliposomes Including Cholesterol, Polysorbate-80, Ceteareth-30, Lecithin, Magnetite, Fractionated Coconut Oil, and Ibuprofen

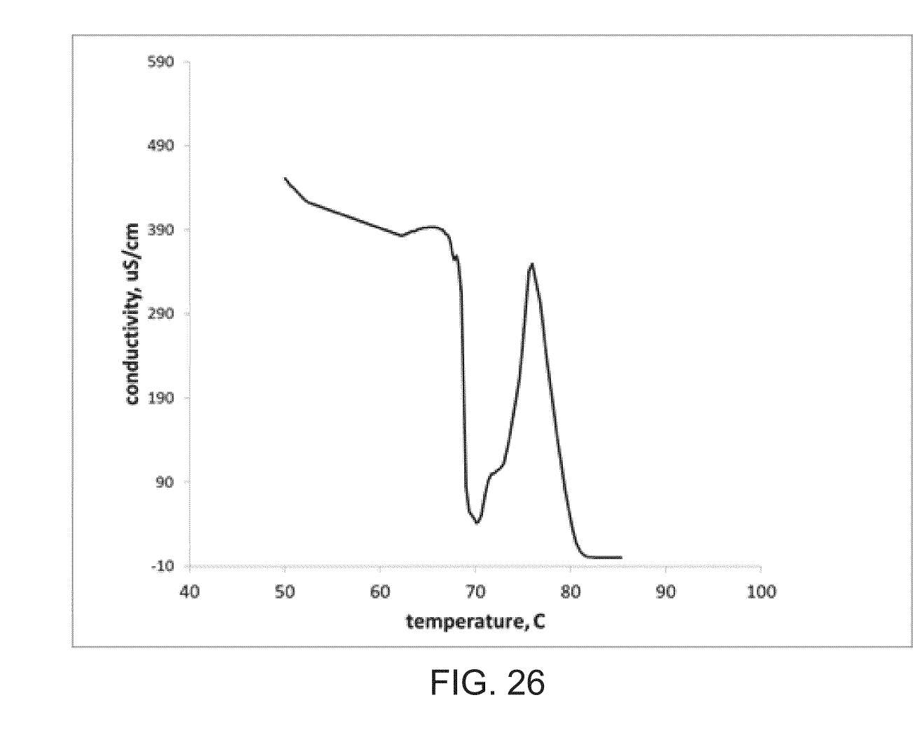

The conductivity of a mixture of 12.0 grams polysorbate-80, 12.0 grams Alcolec XTRA-A lecithin, 45.0 grams fractionated coconut oil, 1.49 grams ceteareth-30, 3.17 grams cholesterol, 4.70 grams of ibuprofen, 5.05 grams of Ferrofluid (FerroTec catalog number EFH1, available from Applied Magnets Superstore, Plano Tex.) and 60.0 grams of distilled water was measured as the mixture cooled from about 80° C. to 62° C. The plot of conductivity (uS/cm) vs. temperature (° C.) is shown in FIG. 26. Because of the dark color of the mixture, it was not possible to determine visually if a microemulsion was present. In comparison to the composition of Example 15, it is inferred that a microemulsion exists in the temperature range of about 76° C. to about 80° C. After measuring conductivity, the sample was reheated to about 90° C. and when at about 79° C., 28,0 grams of the composition was poured into 93 grams of stirring water at about 7° C. to give an aqueous dispersion. The dispersion appeared to be colloidal when added dropwise into distilled water. A magnet on the outside wall of a beaker containing the dispersion caused a darker area to appear. After stirring for about 15 minutes and standing for about 30 minutes, a small amount of dark brown magnetic precipitate settled to the bottom of the container. In a separate experiment, a mixture of 6.0 grams polysorbate-80, 6.1 grams Alcolec XTRA-A lecithin, 22.6 grams fractionated coconut oil, 0.77 grams ceteareth-30, 1.48 grams cholesterol, 2.29 grams of ibuprofen and 30.0 grams of distilled water was heated to about 90° C. and allowed to cool while stirring. The composition was a homogeneous, hazy transparent microemulsion between about 75° C. and about 85° C. After cooling to about 70° C., water that evaporated was replaced, the sample was re-heated, and when stirring at about 95° C., 2.61 grams of Ferrofluid was added. The dark brown mixture was allowed to cool and when at about 80° C., 20.7 grams of microemulsion was poured into 180 grams of stirring cold (7° C.) water to give a deep brown colored magnetoliposomal dispersion. The volume average particle size of the magnetoliposomal dispersion was measured by DLS and found to about 170.2 nm with polydispersity (by the method of cumulants) equal to about −0.019.

### Example 26

### Drug Free Microemulsions and Liposomes Including Cholesterol, Polysorbate-80, Ceteareth-30, Lecithin, Fractionated Coconut Oil, and Octanoic Acid

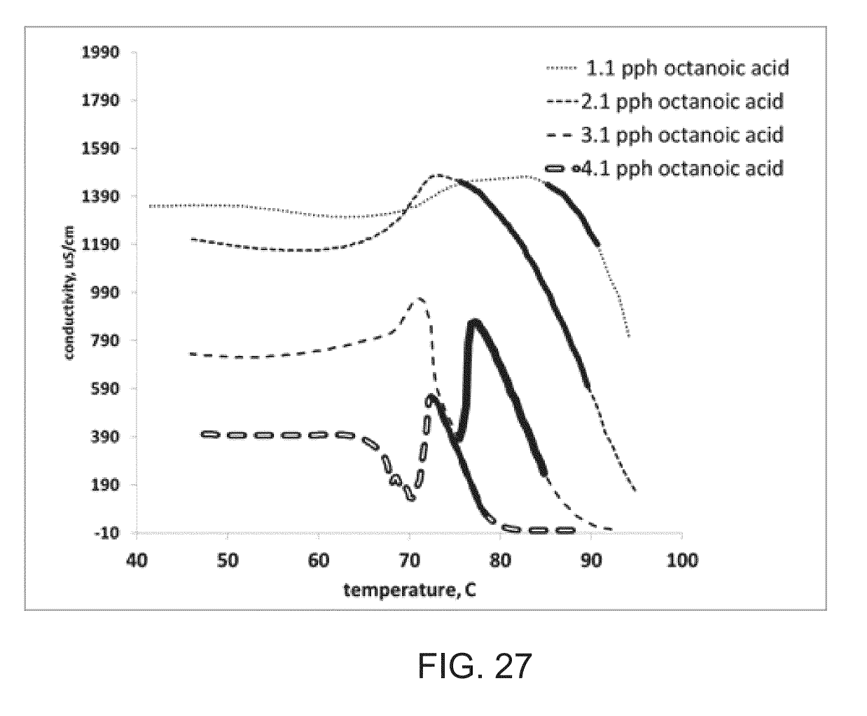

The conductivity of a mixture of 12.1 grams polysorbate-80, 12.2 grams Alcolec XTRA-A lecithin, 50.0 grams fractionated coconut oil, 1.5 grams ceteareth-30, 3.0 grams cholesterol, 1.55 grams octanoic acid and 60.1 grams water was measured as it cooled from about 94° C. to about 42° C. This composition has about 1.1 pph octanoic acid, weight average HLB equal to about 9.4 oil to surfactant ratio (including octanoic acid) equal to about 1.83:1 and HLB polydispersity as WMSD_{HLB} equal to about 5.5. The composition was transparent and hazy between about 65° C. and about 91° C., indicating a microemulsion. After measuring conductivity, water that evaporated was replaced, the sample was re-heated, and an additional 1.47 grams of octanoic acid added to give 2.1 pph octanoic acid, weight average HLB equal to about 9.2, oil to surfactant ratio (including octanoic acid) equal to 1.74:1 and HLB polydispersity as WMSD_{HLB} equal to about 5.5. The composition was transparent and hazy between about 76° C. and about 90° C. After measuring conductivity, water that evaporated was replaced, the sample was re-heated, and an additional 1.53 grams of octanoic acid added to give 3.1 pph octanoic acid, weight average HLB equal to about 9.0, oil to surfactant ratio (including octanoic acid) equal to about 1.65:1 and HLB polydispersity as WMSD_{HLB} equal to about 5.5. The composition was transparent and hazy between about 76° C. and about 85° C. After measuring conductivity, water that evaporated was replaced and the sample was reheated to about 90° C. and allowed to cool with stirring. When it was at about 78° C., 38.1 grams was poured into 160 grams of cold stirring water to give a liposomal dispersion. The specific turbidity of the sample was about 0.33 NTU/ppm. To the remaining mixture, an additional 1.07 grams of octanoic acid was added to give a composition with 4.1 pph octanoic acid, oil to surfactant ratio (including octanoic acid) equal to about 1.58:1 and HLB polydispersity as WMSD_{HLB} equal to about 5.4. Conductivity was measured between about 47° C. and about 88° C., and the composition was a microemulsion between about 72° C. and about 78° C. After measuring conductivity, water that evaporated was replaced, the mixture was reheated, and when at about 78° C., 28.1 grams was poured into 159 grams stirring cold water to give a liposomal dispersion. The specific turbidity of the sample was about 0.54 NTU/ppm, indicating larger particle size liposomes. Plots of plot of conductivity (uS/cm) vs. temperature (° C.) for the four octanoic acid microemulsion compositions are shown in FIG. 27. This example affords a lotion.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B. C. and D, and lastly E” shall he construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance or component fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention.

Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

What is claimed is:

1. A niosomal composition comprising:
one or more high K_{ow} pharmacologically active compounds each independently having a pK_{ow} value greater than about 1.5;
one or more water immiscible oils;
one or more low HLB surfactants each independently having a HLB value of less than 12;
one or more polyethoxylated high HLB surfactants each independently having a HLB value of equal to or greater than 12;
water,
wherein the niosomal composition comprises an external phase and a dispersed phase, and
wherein one or more particles of the dispersed phase comprise a lipid bilayer.

2. The niosomal composition of claim 1, wherein the one or more high K_{ow} pharmacologically active compounds comprise from about 0.05 to about 10 weight percent (%).

3. The niosomal composition of claim 1, wherein the one or more water immiscible oils comprise from about 0.3 to about 70 weight percent (%).

4. The niosomal composition of claim 1, wherein the one or more low HLB surfactants comprise from about 0.15 to about 35 weight percent (%).

5. The niosomal composition of claim 1, wherein the one or more polyethoxylated high HLB surfactants comprise from about 0.15 to about 35 weight percent (%).

6. The niosomal composition of claim 1, wherein the water comprises from 25 to about 99 weight percent (%).

7. The niosomal composition of claim 1, wherein the one or more high K_{ow} pharmacologically active compounds each independently atropine, cortisol, cortisone, diclofenac, diflusinal, docetaxel, dronabinol, estradiol, flurbiprofen, haloperidol, ibuprofen, ketoprofen, lidocaine, naproxen, benzocaine, paclitaxel, penicillin V, prednisone, progesterone, salicylic acid, and sulindac, or a combination thereof.

8. The niosomal composition of claim 1, wherein the one or more high K_{ow} pharmacologically active compounds each independently comprise ibuprofen, naproxen, or a combination thereof.

9. The niosomal composition of claim 1, wherein the one or more water immiscible oils each independently comprise isopropyl myristate, coconut oil, mineral oil, or a combination thereof.

10. The niosomal composition of claim 1, wherein the one or more low HLB surfactants each independently comprise lecithin, sorbitan monostearate; octanoic acid, or a combination thereof.

11. The niosomal composition of claim 1, wherein the one or more polyethoxylated high HLB surfactants each independently comprise laureth 23, polysorbate 80, ceteareth-30, or a combination thereof.

12. A niosomal composition comprising:
one or more high K_{ow} pharmacologically active compounds each independently having a pK_{ow} value greater than about 1.5,
wherein the one or more high K_{ow} pharmacologically active compounds each independently comprise ibuprofen, naproxen, or a combination thereof;
one or more water immiscible oils,
wherein the one or more water immiscible oils each independently comprise isopropyl myristate, coconut oil, mineral oil, or a combination thereof;
one or more low HLB surfactants each independently having a HLB value of less than 12,
wherein the one or more low HLB surfactants each independently comprise lecithin, sorbitan monostearate; octanoic acid, or a combination thereof;
one or more polyethoxylated high HLB surfactants each independently having a HLB value of equal to or greater than 12;
wherein the one or more polyethoxylated high HLB surfactants each independently comprise laureth 23, polysorbate 80, ceteareth-30, or a combination thereof;
water,
wherein the niosomal composition comprises an external phase and a dispersed phase, and
wherein one or more particles of the dispersed phase comprise a lipid bilayer.

13. A niosomal composition of claim 11, comprising:
ibuprofen;
isopropyl myristate;
laureth 23;
sorbitan monostearate;
lecithin;
mineral oil; and
water,
or
ibuprofen;
fractionated coconut oil;
laureth 23;
lecithin; and
water,
or
ibuprofen;
fractionated coconut oil;
lecithin;
polysorbate 80; and
water,
or
ibuprofen;
cholesterol;
polysorbate-80;
ceteareth-30;
lecithin;
fractionated coconut oil; and
water.

14. A niosomal composition of claim 11, comprising:
naproxen;
isopropyl myristate;
laureth 23;
sorbitan monostearate;
lecithin;
mineral oil; and
water,
or
naproxen;
fractionated coconut oil;
laureth 23;
lecithin; and
water,
or
naproxen;
fractionated coconut oil;
lecithin;
polysorbate 80; and
water,
or
naproxen;
cholesterol;
polysorbate-80;
ceteareth-30;
lecithin;
fractionated coconut oil;
octanoic acid; and
water.

15. A method of topically treating a disorder in a patient in need thereof comprising:
administering topically a therapeutically effective amount of a niosomal composition comprising:
one or more high K_{ow} pharmacologically active compounds each independently having a value greater than about 1.5;
one or more water immiscible oils;
one or more low HLB surfactants each independently having a HLB value of less than 12;
one or more polyethoxylated high HLB surfactants each independently having a HLB value of equal to or greater than 12;
water, and
wherein the niosomal composition comprises an external phase and a dispersed phase.

16. The method of claim 14, wherein the disorder comprises pain, inflammation, muscle tightness, muscle spasms, skin ulcerations, scleroderma, eczema, lichen simplex chronicus, rashes, dermatoses, seborrheic dermatitis, psoriasis, atopic dermatitis, or a combination thereof.

17. The method of claim 14, wherein the one or more high pharmacologically active compounds each independently comprise ibuprofen, naproxen, or a combination thereof.

18. The method of claim 14, wherein the one or more water immiscible oils each independently comprise isopropyl myristate, coconut oil, mineral oil, or a combination thereof.

19. The method of claim 14, wherein the one or more low HLB surfactants each independently comprise lecithin, cholesterol, ceteareth-30, sorbitan monostearate; octanoic acid, or a combination thereof.

20. The method of claim 14, wherein the one or more polyethoxylated high HLB surfactant each independently comprise laureth 23, polysorbate 80, or a combination thereof.

21. The method of claim 14, wherein the niosomal composition comprises:
ibuprofen;
isopropyl myristate;
laureth 23;
sorbitan monostearate;
lecithin;
mineral oil; and
water,
or
ibuprofen;
fractionated coconut oil;
laureth 23;
lecithin; and
water,
or
ibuprofen;
fractionated coconut oil;
lecithin;
polysorbate 80; and
water,
or
ibuprofen;
cholesterol;
polysorbate-80;
ceteareth-30;
lecithin;
fractionated coconut oil; and
water,
or naproxen;
isopropyl myristate;
laureth 23;
sorbitan monostearate;
lecithin;
mineral oil; and
water,
or
naproxen;
fractionated coconut oil;
laureth 23;
lecithin; and
water,
or
naproxen;
fractionated coconut oil;
lecithin;
polysorbate 80; and
water,
or
naproxen;
cholesterol;
polysorbate-80;
ceteareth-30;
lecithin;
fractionated coconut oil;
octanoic acid; and
water.
